SEQUENCE LISTING (223 sequences)

SEQ ID NO: 1 - protein, 19 aa, synthetic construct
DWSGYSGSFVQHPELTGLD

SEQ ID NO: 2 - protein, 7 aa, synthetic construct
ETPIRNE

SEQ ID NO: 3 - protein, 15 aa, synthetic construct
FVIREPFISCSHLEC

SEQ ID NO: 4 - protein, 6 aa, synthetic construct
GNFIAP

SEQ ID NO: 5 - protein, 6 aa, synthetic construct
GNLIAP

SEQ ID NO: 6 - protein, 7 aa, synthetic construct
GNLFIAP

SEQ ID NO: 7 - protein, 7 aa, synthetic construct
GNLIFAP

SEQ ID NO: 8 - protein, 8 aa, synthetic construct
HYEECSCY

SEQ ID NO: 9 - protein, 10 aa, synthetic construct
LLTEVETPIR

SEQ ID NO: 10 - protein, 11 aa, synthetic construct
LLTEVETPIRN

SEQ ID NO: 11 - protein, 12 aa, synthetic construct
LLTEVETPIRNE

SEQ ID NO: 12 - protein, 18 aa, synthetic construct
DWSGYSGSFVQHPELTGL

SEQ ID NO: 13 - protein, 9 aa, synthetic construct
EVETPIRNE

SEQ ID NO: 14 - protein, 38 aa, synthetic construct
FLLPEDETPIRNEWGLLTDDETPIRYIKANSKFIGITE

SEQ ID NO: 15 - protein, 64 aa, synthetic construct
GNLFIAPGNLFIAPHYEECSCYHYEECSCYQYIKANSKFIGITEHYEECSCYTPIRNETPIRNE

SEQ ID NO: 16 - protein, 35 aa, synthetic construct
GNLFIAPGNLFIAPQYIKANSKFIGITEGNLFIAP

SEQ ID NO: 17 - protein, 48 aa, synthetic construct
HYEECSCYDWSGYSGSFVQHPELTGLHYEECSCYQYIKANSKFIGITE

SEQ ID NO: 18 - protein, 33 aa, synthetic construct
ITGFAPFSKDNSIRLSAGGDIWVTREPYVSCDP

SEQ ID NO: 19 - protein, 7 aa, synthetic construct
IWGIHHP

SEQ ID NO: 20 - protein, 7 aa, synthetic construct
IWGVHHP

SEQ ID NO: 21 - protein, 8 aa, synthetic construct
IWGVIHHP

SEQ ID NO: 22 - protein, 8 aa, synthetic construct
IWGIVHHP

SEQ ID NO: 23 - protein, 16 aa, synthetic construct
KSCINRCFYVELIRGR

SEQ ID NO: 24 - protein, 27 aa, synthetic construct
LLTEVETPIRNESLLTEVETPIRNEWG

SEQ ID NO: 25 - protein, 13 aa, synthetic construct
LLTEVETPIRNEW

SEQ ID NO: 26 - protein, 14 aa, synthetic construct
LLTEVETPIRNEWG

SEQ ID NO: 27 - protein, 11 aa, synthetic construct
LTEVETPIRNE

SEQ ID NO: 28 - protein, 12 aa, synthetic construct
LTEVETPIRNEW

SEQ ID NO: 29 - protein, 13 aa, synthetic construct
LTEVETPIRNEWG

SEQ ID NO: 30 - protein, 9 aa, synthetic construct
MSLLTEVET

SEQ ID NO: 31 - protein, 10 aa, synthetic construct
MSLLTEVETP

SEQ ID NO: 32 - protein, 11 aa, synthetic construct
MSLLTEVETPI

SEQ ID NO: 33 - protein, 12 aa, synthetic construct
MSLLTEVETPIR

SEQ ID NO: 34 - protein, 13 aa, synthetic construct
MSLLTEVETPIRN

SEQ ID NO: 35 - protein, 14 aa, synthetic construct
MSLLTEVETPIRNE

SEQ ID NO: 36 - protein, 20 aa, synthetic construct
MSLLTEVETPIRNETPIRNE

SEQ ID NO: 37 - protein, 15 aa, synthetic construct
MSLLTEVETPIRNEW

SEQ ID NO: 38 - protein, 16 aa, synthetic construct
MSLLTEVETPIRNEWG

SEQ ID NO: 39 - protein, 24 aa, synthetic construct
MSLLTEVETPIRNEWGCRCNDSSD

SEQ ID NO: 40 - protein, 8 aa, synthetic construct
SLLTEVET

SEQ ID NO: 41 - protein, 11 aa, synthetic construct
SLLTEVETPIR

SEQ ID NO: 42 - protein, 13 aa, synthetic construct
SLLTEVETPIRNE

SEQ ID NO: 43 - protein, 14 aa, synthetic construct
SLLTEVETPIRNEW

SEQ ID NO: 44 - protein, 15 aa, synthetic construct
SLLTEVETPIRNEWG

SEQ ID NO: 45 - protein, 21 aa, synthetic construct
SLLTEVETPIRNEWGTPIRNE

SEQ ID NO: 46 - protein, 27 aa, synthetic construct
SLLTEVETPIRNEWGTPIRNETPIRNE

SEQ ID NO: 47 - protein, 33 aa, synthetic construct
SLLTEVETPIRNEWGTPIRNETPIRNETPIRNE

SEQ ID NO: 48 - protein, 25 aa, synthetic construct
SLLTEVETPIRNEWGLLTEVETPIR

SEQ ID NO: 49 - protein, 10 aa, synthetic construct
TEVETPIRNE

SEQ ID NO: 50 - protein, 6 aa, synthetic construct
TPIRNE

SEQ ID NO: 51 - protein, 8 aa, synthetic construct
VETPIRNE

SEQ ID NO: 52 - protein, 52 aa, synthetic construct
VTREPYVSCDPKSCINRCFYVELIRGRVTREPYVSCDPWYIKANSKFIGITE

SEQ ID NO: 53 - protein, 6 aa, synthetic construct
WGIHHP

SEQ ID NO: 54 - protein, 6 aa, synthetic construct
WGVHHP

SEQ ID NO: 55 - protein, 7 aa, synthetic construct
WGVIHHP

SEQ ID NO: 56 - protein, 7 aa, synthetic construct
WGIVHHP

SEQ ID NO: 57 - protein, 8 aa, synthetic construct
YIWGIHHP

SEQ ID NO: 58 - protein, 8 aa, synthetic construct
YIWGVHHP

SEQ ID NO: 59 - protein, 9 aa, synthetic construct
YIWGVIHHP

SEQ ID NO: 60 - protein, 9 aa, synthetic construct
YIWGIVHHP

SEQ ID NO: 61 - protein, 14 aa, synthetic construct
QYIKANSKFIGITE

SEQ ID NO: 62 - protein, 15 aa, synthetic construct
PIRNEWGCRCNDSSD

SEQ ID NO: 63 - protein, 22 aa, synthetic construct
GNLFIAPWGVIHHPHYEECSCY

SEQ ID NO: 64 - protein, 10 aa, synthetic construct
CAGAGNFIAP

SEQ ID NO: 65 - protein, 10 aa, synthetic construct
CAGAGNLIAP

SEQ ID NO: 66 - protein, 11 aa, synthetic construct
CAGAGNLFIAP

SEQ ID NO: 67 - protein, 10 aa, synthetic construct
CAGAWGVHHP

SEQ ID NO: 68 - protein, 10 aa, synthetic construct
CAGAWGIHHP

SEQ ID NO: 69 - protein, 11 aa, synthetic construct
CAGAWGVIHHP

SEQ ID NO: 70 - protein, 11 aa, synthetic construct
CAGAWGIVHHP

SEQ ID NO: 71 - protein, 13 aa, synthetic construct
GNLIAPWGVIHHP

SEQ ID NO: 72 - protein, 17 aa, synthetic construct
CAGAGNLIAPWGVIHHP

SEQ ID NO: 73 - protein, 14 aa, synthetic construct
GNLFIAPWGVIHHP

SEQ ID NO: 74 - protein, 18 aa, synthetic construct
CAGAGNLFIAPWGVIHHP

SEQ ID NO: 75 - protein, 8 aa, synthetic construct
HYEECSCY

SEQ ID NO: 76 - protein, 12 aa, synthetic construct
CAGAHYEECSCY

SEQ ID NO: 77 - protein, 26 aa, synthetic construct
CAGAGNLFIAPWGVIHHPHYEECSCY

SEQ ID NO: 78 - protein, 28 aa, synthetic construct
GNLFIAPWGVIHHPGNLFIAPWGVIHHP

SEQ ID NO: 79 - protein, 32 aa, synthetic construct
CAGAGNLFIAPWGVIHHPGNLFIAPWGVIHHP

SEQ ID NO: 80 - protein, 22 aa, synthetic construct
HYEECSCYGNLFIAPWGVIHHP

SEQ ID NO: 81 - protein, 22 aa, synthetic construct
GNLFIAPHYEECSCYWGVIHHP

SEQ ID NO: 82 - protein, 39 aa, synthetic construct
SLLTEVETPIRNEWGLLTEVETPIRQYIKANSKFIGITE

SEQ ID NO: 83 - protein, 35 aa, synthetic construct; identical to SEQ ID NO: 16

SEQ ID NO: 84 - protein, 48 aa, synthetic construct; identical to SEQ ID NO: 17

SEQ ID NO: 85 - protein, 52 aa, synthetic construct
VTREPYVSCDPKSCINRCFYVELIRGRVTREPYVSCDPQYIKANSKFIGITE

SEQ ID NO: 86 - protein, 18 aa, synthetic construct
DWSGYSGSFVQHPELTGL

SEQ ID NO: 87 - protein, 33 aa, synthetic construct; identical to SEQ ID NO: 18

SEQ ID NO: 88 - protein, 16 aa, synthetic construct
KSCINRCFYVELIRGR

SEQ ID NO: 89 - protein, 12 aa, synthetic construct
GNLFIAPRYAFA

SEQ ID NO: 90 - protein, 16 aa, synthetic construct
CAGAGNLFIAPRYAFA

SEQ ID NO: 91 - protein, 11 aa, synthetic construct
GNLVVPRYAFA

SEQ ID NO: 92 - protein, 15 aa, synthetic construct
CAGAGNLVVPRYAFA

SEQ ID NO: 93 - protein, 11 aa, synthetic construct
GNLIAPRYAFA

SEQ ID NO: 94 - protein, 15 aa, synthetic construct
CAGAGNLIAPRYAFA

SEQ ID NO: 95 - protein, 6 aa, synthetic construct
GNLVVP

SEQ ID NO: 96 - protein, 10 aa, synthetic construct
CAGAGNLVVP

SEQ ID NO: 97 - protein, 15 aa, synthetic construct
FVIREPFISCSHLEC

SEQ ID NO: 98 - protein, 19 aa, synthetic construct
CAGAFVIREPFISCSHLEC

SEQ ID NO: 99 - protein, 22 aa, synthetic construct
GNLFIAPWGVIHHPHYEECSCY

SEQ ID NO: 100 - protein, 36 aa, synthetic construct
GNLFIAPWGVIHHPHYEECSCYQYIKANSKFIGITE

SEQ ID NO: 101 - protein, 36 aa, synthetic construct
QYIKANSKFIGITEGNLFIAPWGVIHHPHYEECSCY

SEQ ID NO: 102 - protein, 7 aa, synthetic construct
HVEECSY

SEQ ID NO: 103 - protein, 6 aa, synthetic construct
WFIHHP

SEQ ID NO: 104 - protein, 11 aa, synthetic construct
DLWSYNAELLV

SEQ ID NO: 105 - protein, 11 aa, synthetic construct
DIWTYNAELLV

SEQ ID NO: 106 - skipped

SEQ ID NO: 107 - protein, 7 aa, synthetic construct
YPKCDRA

SEQ ID NO: 108 - protein, 9 aa, synthetic construct
WDYPKCDRA

SEQ ID NO: 109 - protein, 23 aa, synthetic construct
SLDQINVTFLDLEYEMKKLEESY

SEQ ID NO: 110 - protein, 23 aa, synthetic construct
SLDQINVTFLDLEYEMKKLEESY

SEQ ID NO: 111 - protein, 37 aa, synthetic construct
SLDQINVTFLDLEYEMKKLEESYQYIKANSKFIGITE

SEQ ID NO: 112 - protein, 9 aa, synthetic construct
WDYPKCDRA

SEQ ID NO: 113 - protein, 23 aa, synthetic construct
WDYPKCDRAQYIKANSKFIGITE

SEQ ID NO: 114 - protein, 46 aa, synthetic construct
WDYPKCDRASLDQINVTFLDLEYEMKKLEESYQYIKANSKFIGITE

SEQ ID NO: 115 - protein, 41 aa, synthetic construct
WDYPKCDRATEVETPIRNEHYEECSCYQYIKANSKFIGITE

SEQ ID NO: 116 - protein, 37 aa, synthetic construct; identical to SEQ ID NO: 111

SEQ ID NO: 117 - protein, 9 aa, synthetic construct
WDYPKCDRA

SEQ ID NO: 118 - protein, 7 aa, synthetic construct
YPKCDRA

SEQ ID NO: 119 - protein, 8 aa, synthetic construct
ARDLICAQ

SEQ ID NO: 120 - protein, 14 aa, synthetic construct
KWPWYIWLGFIAGL

SEQ ID NO: 121 - protein, 8 aa, synthetic construct
ENQKLIAN

SEQ ID NO: 122 - protein, 30 aa, synthetic construct
ARDLICAQKWPWYIWLGFIAGLENQKLIAN

SEQ ID NO: 123 - protein, 16 aa, synthetic construct
ENQKLIANARDLICAQ

SEQ ID NO: 124 - protein, 25 aa, synthetic construct
WDYPKCDRAENQKLIANARDLICAQ

SEQ ID NO: 125 - protein, 31 aa, synthetic construct
WDYPKCDRAENQKLIANKWPWYIWLGFIAGL

SEQ ID NO: 126 - protein, 39 aa, synthetic construct
ARDLICAQENQKLIANWDYPKCDRAQYIKANSKFIGITE

SEQ ID NO: 127 - protein, 76 aa, synthetic construct
KWPWYIWLGFIAGLWDYPKCDRAQYIKANSKFIGITEARDLICAQENQKLIANWDYPKCDRAQYIKANSKFIGITE

SEQ ID NO: 128 - protein, 69 aa, synthetic construct
ARDLICAQENQKLIANQYIKANSKFIGITEARDLICAQENQKLIANWDYPKCDRAQYIKANSKFIGITE

SEQ ID NO: 129 - protein, 80 aa, synthetic construct
WDYPKCDRATEVETPIRNEHYEECSCYQYIKANSKFIGITEARDLICAQENQKLIANWDYPKCDRAQYIKANSKFIGITE

SEQ ID NO: 130 - protein, 39 aa, synthetic construct
HYEECSCYWDYPKCDRAVETPIRNEQYIKANSKFIGITE

SEQ ID NO: 131 - protein, 40 aa, synthetic construct
ENQKLIANTEVETPIRNEHYEECSCYQYIKANSKFIGITE

SEQ ID NO: 132 - protein, 13 aa, synthetic construct
AEKAGGGGGAEKA

SEQ ID NO: 133 - protein, 19 aa, synthetic construct
AEKAEKAGGGGGAEKAEKA

SEQ ID NO: 134 - protein, 26 aa, synthetic construct
QYIKANSKFIGITEAEKAGGGGAEKA

SEQ ID NO: 135 - protein, 27 aa, synthetic construct
AEKAGGGGGAEKAQYIKANSKFIGITE

SEQ ID NO: 136 - protein, 4 aa, synthetic construct
AEKA

SEQ ID NO: 137 - protein, 9 aa, synthetic construct
AEKAGGGGG

SEQ ID NO: 138 - protein, 14 aa, synthetic construct
QYIKANSKFIGITE

SEQ ID NO: 139 - protein, 5 aa, synthetic construct
GGGGG

SEQ ID NO: 140 - protein, 20 aa, synthetic construct
SEFAYGSFVRTVSLPVGADE

SEQ ID NO: 141 - protein, 42 aa, synthetic construct
SEFAYGSFVRTVSLPVGADEGNLFIAPWGVIHHPHYEECSCY

SEQ ID NO: 142 - protein, 12 aa, synthetic construct
HSFKWLDSPRLR

SEQ ID NO: 143 - protein, 12 aa, synthetic construct
ISLTEWSMWYRH

SEQ ID NO: 144 - protein, 15 aa, synthetic construct
WRMYFSHRHAHLRSP

SEQ ID NO: 145 - protein, 15 aa, synthetic construct
WHWRHRIPLQLAAGR

SEQ ID NO: 146 - protein, 22 aa, synthetic construct
GNLFIAPWGVIHHPHYEECSCY

SEQ ID NO: 147 - protein, 20 aa, synthetic construct
SEFAYGSFMRSVTLPPGADE

SEQ ID NO: 148 - protein, 20 aa, synthetic construct
SEFAYGSFVRTVSLPVGADE

SEQ ID NO: 149 - protein, 33 aa, synthetic construct
AEKAGGGGGAEKASEFAYGSFVRTVSLPVGADE

SEQ ID NO: 150 - protein, 47 aa, synthetic construct
AEKAGGGGGAEKASEFAYGSFVRTVSLPVGADEQYIKANSKFIGITE

SEQ ID NO: 151 - protein, 33 aa, synthetic construct
SEFAYGSFVRTVSLPVGADEAEKAGGGGGAEKA

SEQ ID NO: 152 - protein, 38 aa, synthetic construct
AEKAGGGGGSEFAYGSFVRTVSLPVGADEGGGGGAEKA

SEQ ID NO: 153 - protein, 52 aa, synthetic construct
AEKAGGGGGSEFAYGSFVRTVSLPVGADEGGGGGAEKAQYIKANSKFIGITE

SEQ ID NO: 154 - protein, 43 aa, synthetic construct
WRMYFSHRHAHLRSPGGGGGAEKAGGGGGQYIKANSKFIGITE

SEQ ID NO: 155 - protein, 52 aa, synthetic construct
WHWRHRIPLQLAGRAEKAGGGGGWRMYFSHRHAHLRSPQYIKANSKFIGITE

SEQ ID NO: 156 - protein, 41 aa, synthetic construct; identical to SEQ ID NO: 115

SEQ ID NO: 157 - protein, 7 aa, synthetic construct
GNLFIAP

SEQ ID NO: 158 - protein, 7 aa, synthetic construct
WGVIHHP

SEQ ID NO: 159 - protein, 8 aa, synthetic construct
HYEECSCY

SEQ ID NO: 160 - protein, 21 aa, synthetic construct
YFPLQSYGFQPTNGVGYQPYR

SEQ ID NO: 161 - protein, 35 aa, synthetic construct
YFPLQSYGFQPTNGVGYQPYRQYIKANSKFIGITE

SEQ ID NO: 162 - protein, 34 aa, synthetic construct
YQAGSTPCNGVEGFNCYFPLQYIKANSKFIGITE

SEQ ID NO: 163 - protein, 21 aa, synthetic construct
YQAGSTPCNGVEGFNCYFPLQ

SEQ ID NO: 164 - protein, 7 aa, synthetic construct
ETPIRNE

SEQ ID NO: 165 - protein, 10 aa, synthetic construct
TEVETPIRNE

SEQ ID NO: 166 - protein, 25 aa, synthetic construct
SLLTEVETPIRNEWGLLTEVETPIR

SEQ ID NO: 167 - protein, 40 aa, synthetic construct; identical to SEQ ID NO: 131

SEQ ID NO: 168 - protein, 9 aa, synthetic construct
WDYPKCDRA

SEQ ID NO: 169 - protein, 23 aa, synthetic construct
WDYPKCDRAQYIKANSKFIGITE

SEQ ID NO: 170 - protein, 41 aa, synthetic construct; identical to SEQ ID NO: 115

SEQ ID NO: 171 - protein, 8 aa, synthetic construct
ENQKLIAN

SEQ ID NO: 172 - protein, 40 aa, synthetic construct; identical to SEQ ID NO: 131

SEQ ID NO: 173 - protein, 19 aa, synthetic construct
NPDPNANPNVDPNANGGGG

SEQ ID NO: 174 - protein, 13 aa, synthetic construct
RKSIHLGPGRAFY

SEQ ID NO: 175 - protein, 13 aa, synthetic construct
KKGIAIGPGRTLY

SEQ ID NO: 176 - protein, 13 aa, synthetic construct
RKSIRIGPGQAFY

SEQ ID NO: 177 - protein, 13 aa, synthetic construct
RKRIRVGPGQTVY

SEQ ID NO: 178 - protein, 9 aa, synthetic construct
CATGIAVAG

SEQ ID NO: 179 - protein, 10 aa, synthetic construct
YYYYYGMDVW

SEQ ID NO: 180 - protein, 14 aa, synthetic construct
CATGYSSSWYFDYW

SEQ ID NO: 181 - protein, 15 aa, synthetic construct
CAKGYSYGYNWFDSW

SEQ ID NO: 182 - protein, 12 aa, synthetic construct [sequence omitted]

SEQ ID NO: 183 - protein, 23 aa, synthetic construct
CATGIAVAGQYIKANSKFIGITE

SEQ ID NO: 184 - protein, 24 aa, synthetic construct
YYYYYGMDVWQYIKANSKFIGITE

SEQ ID NO: 185 - protein, 28 aa, synthetic construct
CATGYSSSWYFDYWQYIKANSKFIGITE

SEQ ID NO: 186 - protein, 29 aa, synthetic construct
CAKGYSYGYNWFDSWQYIKANSKFIGITE

SEQ ID NO: 187 - protein, 26 aa, synthetic construct
CQQYNNWPPLTFQYIKANSKFIGITE

SEQ ID NO: 188 - protein, 36 aa, synthetic construct
ARDLICAQCATGYSSSWYFDYWQYIKANSKFIGITE

SEQ ID NO: 189 - protein, 53 aa, synthetic construct
WDYPKCDRATEVETPIRNEHYEECSCYCQQYNNWPPLTFQYIKANSKFIGITE

SEQ ID NO: 190 - protein, 58 aa, synthetic construct
WDYPKCDRATEVETPIRNEARDLICAQENQKLIANCATGIAVAGQYIKANSKDIGITE

SEQ ID NO: 191 - protein, 53 aa, synthetic construct
WDYPKCDRAENQKLIANARDLICAQCATGYSSSWYFDYWQYIKANSKFIGITE

SEQ ID NO: 192 - protein, 26 aa, synthetic construct
CQQYNNWPPLTFQYIKANSKFIGITE

SEQ ID NO: 193 - protein, 36 aa, synthetic construct; identical to SEQ ID NO: 188

SEQ ID NO: 194 - protein, 53 aa, synthetic construct; identical to SEQ ID NO: 189

SEQ ID NO: 195 - protein, 58 aa, synthetic construct; identical to SEQ ID NO: 190

SEQ ID NO: 196 - protein, 53 aa, synthetic construct; identical to SEQ ID NO: 191

SEQ ID NO: 197 - protein, 7 aa, synthetic construct
QEINSSY

SEQ ID NO: 198 - protein, 7 aa, synthetic construct
APPHALS

SEQ ID NO: 199 - protein, 7 aa, synthetic construct
VVPTPPY

SEQ ID NO: 200 - protein, 7 aa, synthetic construct
SMPNPMV

SEQ ID NO: 201 - protein, 7 aa, synthetic construct
GLQQVLL

SEQ ID NO: 202 - protein, 7 aa, synthetic construct
ELAPDSP

SEQ ID NO: 203 - protein, 21 aa, synthetic construct
QEINSSYQYIKANSKFIGITE

SEQ ID NO: 204 - protein, 21 aa, synthetic construct
APPHALSQYIKANSKFIGITE

SEQ ID NO: 205 - protein, 21 aa, synthetic construct
VVPTPPYQYIKANSKFIGITE

SEQ ID NO: 206 - protein, 45 aa, synthetic construct
QEINSSYAEKAGGGGGWRMYFSHRHAHLRSPQYIKANSKFIGITE

SEQ ID NO: 207 - protein, 45 aa, synthetic construct
APPHALSAEKAGGGGGWRMYFSHRHAHLRSPQYIKANSKFIGITE

SEQ ID NO: 208 - protein, 45 aa, synthetic construct
VVPTPPYAEKAGGGGGWRMYFSHRHAHLRSPQYIKANSKFIGITE

SEQ ID NO: 209 - protein, 46 aa, synthetic construct
AEAKAGGGGGWRMYFSHRHAHLRSPQEINSSYQYIKANSKFIGITE

SEQ ID NO: 210 - protein, 45 aa, synthetic construct
WRMYFSHRHAHLRSPAPPHALSAEKAGGGGGQYIKANSKFIGITE

SEQ ID NO: 211 - protein, 44 aa, synthetic construct
QYIKANSKFIGITEWRMYFSHRHAHLRSAEKAGGGGGVVPTPPY

SEQ ID NO: 212 - protein, 57 aa, synthetic construct
QEINSSYAEKAGGGGGWRMYFSHRHAHLRSPGFSVITGAAMFEQYIKANSKFIGITE

SEQ ID NO: 213 - protein, 39 aa, synthetic construct; identical to SEQ ID NO: 82

SEQ ID NO: 214 - protein, 19 aa, synthetic construct
NPDPNANPNVDPNANGGGC

SEQ ID NO: 215 - protein, 24 aa, synthetic construct
NMMRFTSQPPNNNMMNYIMDPRTH

SEQ ID NO: 216 - protein, 12 aa, synthetic construct
  VARIANT 8: note = Any amino acid
STLNYMYXAHPF

SEQ ID NO: 217 - protein, 12 aa, synthetic construct
ISLSNIVDSQTP

SEQ ID NO: 218 - protein, 12 aa, synthetic construct
GFSVITGAAMFE

SEQ ID NO: 219 - protein, 46 aa, synthetic construct
NPDPNANPNVDPNANGGGCRKSIHLGPGRAFYQYIKANSKFIGITE

SEQ ID NO: 220 - protein, 50 aa, synthetic construct
  VARIANT 8: note = Any amino acid
STLNYMYXAHPFWRMYFSHRHAHLRSPGGGGGAEKAQYIKANSKFIGITE

SEQ ID NO: 221 - protein, 35 aa, synthetic construct
NMMRFTSQPPNNGGGGGAEKAQYIKANSKFIGITE

SEQ ID NO: 222 - protein, 58 aa, synthetic construct
NPDPNANPNVDPNANGGGCHSFKWLDSPRLRRKSIRIGPGQAFYQYIKANSKFIGITE

SEQ ID NO: 223 - protein, 55 aa, synthetic construct
AEKAGGGGGHSFKWLDSPRLRSEFAYGSFVRTVSLPVGADEQYIKANSKFIGITE

(19) United States
(12) Patent Application Publication
(10) Pub. No.: US 2024/0091331 A1
(43) Pub. Date: Mar. 21, 2024

(54) Vaccines and Antibodies for the Treatment and Prevention of Microbial Infections

(71) Applicant: Longhorn Vaccines and Diagnostics, LLC, Bethesda, MD (US)

(72) Inventors: Gerald W. Fischer, Bethesda, MD (US); Clara J. Sei, Germantown, MD (US); Jeffrey D. Fischer, Washington, DC (US)

(73) Assignee: Longhorn Vaccines and Diagnostics, LLC, Bethesda, MD (US)

(21) Appl. No.: 18/388,952

(22) Filed: Nov. 13, 2023

Related U.S. Application Data
(63) Continuation-in-part of application No. 18/380,479, filed on Oct. 16, 2023.
Application No. 18/380,479 is a continuation-in-part of application No. 18/366,915, filed on Aug. 8, 2023.
Application No. 18/366,915 is a continuation-in-part of application No. 17/985,296, filed on Nov. 11, 2022.
(60) Provisional application No. 63/538,648, filed on Sep. 15, 2023.
(60) Provisional application No. 63/416,823, filed on Oct. 17, 2022.
(60) Provisional application No. 63/416,219, filed on Oct. 14, 2022.
(60) Provisional application No. 63/396,286, filed on Aug. 9, 2022.
(60) Provisional application No. 63/333,780, filed on Apr. 22, 2022.
(60) Provisional application No. 63/278,759, filed on Nov. 12, 2021.

(51) Int. Cl. A61K 39/04; A61K 39/085; A61P 31/06; C07K 14/31; C07K 14/35

(52) U.S. Cl. CPC A61K 39/04; A61K 39/085; A61P 31/06; C07K 14/31; C07K 14/35; A61K 2039/55566

(57) ABSTRACT

The invention relates to compositions and peptides or peptide sequences that induce an immune response in an animal or a mammal that is protective against infection by one or more pathogens. In addition, the invention relates to immunogenic composition and vaccines comprising compositions and peptide sequences and to method for treating and preventing an infection in animals and mammals such as humans and antibodies.

### REFERENCE TO RELATED APPLICATIONS

This application claims priority to U.S. application Ser. No. 18/380,479, filed Oct. 16, 2023, which claims priority U.S. Provisional Application No. 63/538,648, filed Sep. 15, 2023, U.S. Provisional Application No. 63/416,823, filed Oct. 17, 2022, and U.S. Provisional Application No. 63/416,219, filed Oct. 14, 2022, to U.S. application Ser. No. 18/366,915 filed Aug. 8, 2023, which claims priority to U.S. Provisional Application No. 63/396,286 filed Aug. 9, 2022, and to U.S. application Ser. No. 17/985,296 filed Nov. 11, 2022, which claims priority to U.S. Provisional Application No. 63/333,780 filed Apr. 22, 2022, and U.S. Provisional Application No. 63/278,759 filed Nov. 12, 2021, the entirety of each of which is incorporated by reference.

### SEQUENCE LISTING

The instant application contains a Sequence Listing submitted electronically in XML format and is hereby incorporated by reference in its entirety. Said XML copy, created on Oct. 25, 2023, is named 3022_057_US_SL.xml and is 197,162 bytes in size.

### BACKGROUND

### 1 Field of the Invention

The present invention is directed to composite antigens and vaccines composed of a plurality of epitopes of one or more pathogens, and to tools and methods for generating an immune response. In particular, the invention is directed to compositions comprising microbial specific peptides and/or nucleic acid sequences as vaccines for the treatment and prevention of microbial diseases.

### 2 Description of the Background

Respiratory viruses cause severe infections in children and adults. For example, influenza viruses are etiologic agents for a contagious respiratory illness (commonly referred to as the flu) that primarily affects humans and other vertebrates. Influenza is highly infectious and an acute respiratory disease that has plagued the human race since ancient times. Infection is characterized by recurrent annual epidemics and periodic major worldwide pandemics. Influenza virus infection can cause mild to severe illness and can even lead to death. Every year in the United States, 5 to 20 percent of the population, on average, contracts the flu with more than 200,000 hospitalizations from complications and over 36,000 deaths. Because of the high disease-related morbidity and mortality, direct and indirect social economic impacts of influenza are enormous. Four pandemics occurred in the last century, together causing tens of millions of deaths worldwide.

The CDC and the leading authorities on disease prevention in the world recommend the single best way of preventing a viral respiratory infection in humans is through regular vaccinations. Vaccines for many respiratory viruses if available, would be useful to prevent much human disease and suffering. Conventional influenza are not ideal because the vaccines typically target the immunodominant protein HA. These vaccines have not been universally protective or 100 percent effective at preventing the disease. Antigenic shift prevents flu vaccines from being universally protective or from maintaining effectiveness over many years. The ineffectiveness of conventional vaccines may also be due, in part, to antigenic drift and the resulting variation within antigenic portions of the HA protein most commonly recognized by the immune system. As a result, many humans may find themselves susceptible to the flu virus without an effective method of treatment available since influenza is constantly improving its resistant to current treatments. This scenario is particularly concerning with respect to the H5N1 virus, which is highly virulent but for which there is currently no widely available commercial vaccine to immunize susceptible human populations.

Currently, flu vaccines are reformulated each year due to the yearly emergence of new strains, and generally induce limited immunity. In addition, to achieve a protective immune response, some vaccines are administered with high doses of antigen. This is particularly true for H5N1 vaccines. In addition, influenza vaccines, including H5N1 vaccines, typically present epitopes in the same order as the epitopes are found in nature, generally presenting as whole-viral proteins; consequently, relatively large amounts of protein are required to make an effective vaccine. As a result, each administration includes an increased cost associated with the dose amount, and there is increased difficulty in manufacturing enough doses to vaccinate the general public. Further, the use of larger proteins elevates the risk of undesirable immune responses in the recipient host.

Approximately one third of the world population is infected with Mycobacterium tuberculosis (MTB). Current treatment includes a long course of antibiotics and often requires quarantining of the patient. Resistance is common and an ever-increasing problem, as is the ability to maintain the quarantine of infected patients. Present vaccines include BCG which is prepared from a strain of attenuated (virulence-reduced) live bovine tuberculosis Bacillus, Mycobacterium bovis, and live non-MTB organisms. BCG carries substantial associated risks, especially in immune compromised individuals, and has proved to be only modestly effective and for limited periods. It is generally believed that a humoral response to infection by MTB is ineffective and optimal control of infection must involve activation of T cells and macrophages. As MTB is a human pathogen, research on MTB is often conducted using Mycobacterium smegmatis, which is considered sufficiently similar, but is not pathogenic to humans.

In addition, HIV and malaria continue to infect many people causing suffering and death across the globe. Effective vaccines are presently unavailable and greatly needed.

Within an immune response, T cells are important tools of the immune system and a major source of the cascade of cytokines that occurs following an immune response. Two of the principle forms of T cells are identified by the presence of the cell surface molecules CD4 and CD8. T cells that express CD4 are generally referred to as helper T cells. T helper cells include the subsets Th1 and Th2, and the cytokines they produce are known as Th1-type cytokines and Th2-type cytokines, both sets of which are of critical importance in developing an immune response. The Th1-type cytokines produce a pro-inflammatory response stimulating the opsonization of intracellular parasites, basically the humoral immune response. Interferon gamma is one of the principal Th1 cytokines. The Th2-type cytokines include interleukins 4, 5, 10 and 13, which are closely associated with the promotion of a cellular immune response. Against an infection, a balanced Th1 and Th2 response is most desired.

Protective anti-microbial vaccines are greatly needed that provide protection against or treatment of infection by multiple different microbes including different serotypes, species, and genus of virus, bacteria, fungus, and/or parasites. It is further needed that such vaccines be efficiently and economically produced.

### SUMMARY OF THE INVENTION

The present invention provides new and useful compositions, as well as tools and methods directed to immunogenic compositions, vaccines and antibodies against one or more pathogens for treating and/or preventing infections in mammals such as humans, a viral, bacterial, fungal, or parasitic infection and enhancing the immune system of a patient.

One embodiment of the invention is directed to peptides containing one or more and preferably multiple viral, bacterial, fungal, and/or parasitic epitopes and/or composite peptide antigens or composite epitopes. Peptides of the invention may comprise multiple viral epitopes, bacterial epitopes, and/or parasitic epitopes, or preferably combination of different epitopes of different microbes. The peptides may be part of an immunogenic composition which may optionally contain an adjuvant such as, for example, Freund's, a liposome, saponin, lipid A, squalene, and derivatives and combinations thereof. Preferred adjuvants include, for example, AS01 (Adjuvant System 01) which is a liposome-based adjuvant which comprises QS-21 (a saponin fraction extracted from Quillaja saponaria Molina), and 3-O-desacyl-4′-monophosphoryl lipid A (MPL; a non-toxic derivative of the lipopolysaccharide from Salmonella minnesota) and on occasion a ligand such as a toll-like receptor (e.g., TLR4), AS01b which is a component of the adjuvant Shingrix, ALF (Army Liposome Formulation) which comprises liposomes containing saturated phospholipids, cholesterol, and/or monophosphoryl lipid A (MPLA) as an immunostimulant. ALF has a safety and a strong potency. AS01 is included in the malaria vaccine RTS,S (MOSQUIRIX®). ALF modifications and derivatives include, for example, ALF adsorbed to aluminum hydroxide (ALFA), ALF containing QS21 saponin (ALFQ), and ALFQ adsorbed to aluminum hydroxide (ALFQA). A preferred adjuvant formulation comprises Freund's adjuvant, a liposome, saponin, lipid A, squalene, unilamellar liposomes having a liposome bilayer that comprises at least one phosphatidylcholine (PC) and/or phosphatidylglycerol (PG), as phospholipids, which may be dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), and/or distearyl phosphatidylglycerol (DSPG), a cholesterol, a monophosphoryl lipid A (MPLA), and a saponin. Preferably, the immunogenic composition is a vaccine that treats or prevents a viral, bacterial or parasitic infection in humans, mammals and other animals including but not limited to porcine and avian species.

Another embodiment of the invention comprises a composite peptide containing one or more microbial epitopes and/or composite antigens or epitopes, and one or more T cell stimulating epitopes. The T cell stimulating epitope is obtained or derived from tetanus toxin, tetanus toxin heavy chain proteins, diphtheria toxoid, cross reactive material (CRM and CRM₁₉₇), synthesized or recombinantly produced CRM, tetanus toxoid, Pseudomonas exoprotein A, Pseudomonas aeruginosa toxoid, Bordetella pertussis toxoid, Clostridium perfringens toxoid, Escherichia coli heat-labile toxin B subunit, Neisseria meningitidis outer membrane complex, Hemophilus influenzae protein D, Flagellin Fli C, Horseshoe crab Haemocyanin, and/or a fragment, derivative, or modification thereof. Preferably the T cell stimulating epitope is at the N-terminus or the C-terminus of the peptide. Peptides of the invention may comprise multiple microbial epitopes and/or multiple T cell stimulating epitopes. Composite peptides may be part of an immunogenic composition which may optionally contain an adjuvant such as, for example, Freund's, ALFQ, ALFQA, ALFA, AS01, AS01b, a liposome, saponin, lipid A, squalene, oil in water emulsion and derivatives and combinations thereof. Preferably, the immunogenic composition is a vaccine that treats and/or prevents a viral, bacterial or parasitic infection in humans, mammals and other animals.

Another embodiment of the invention is directed to antibodies including polyclonal and monoclonal antibodies that bind to the peptide and sequences as disclosed herein including composite peptide epitopes, viral epitopes, bacterial epitopes, parasitic epitopes, fungal epitopes or viral peptide epitopes. Antibodies of this disclosure include mammalian antibodies (e.g., murine, caprine), and human and humanized antibodies.

Another embodiment of the invention is directed to hybridoma cells and cell lines that express monoclonal antibodies as disclosed herein.

Other embodiments and advantages of the invention are set forth in part in the description, which follows, and in part, may be obvious from this description, or may be learned from the practice of the invention.

### DESCRIPTION OF THE FIGURES

FIG. 1A Antisera Binding Titers to Influenza A virus (H1N1, H3N2) and Influenza B virus (Yamagata lineage) from sera of ICR mice immunized intramuscularly (IM) with three different doses of unconjugated composite peptide vaccine (Flu Pep5906+Flu Pep11) with ADDAVAX™ (an oil-in-water nano-emulsion) adjuvant.

FIG. 1B Antisera Binding Titers to Influenza A virus (H5N1) and Influenza B virus (Yamagata lineage) from sera of ICR mice immunized intramuscularly (IM) with three different doses of unconjugated composite peptide vaccine (Flu Pep5906 +Flu Pep11) with ADDAVAX™ adjuvant.

FIG. 2 Antisera Neutralizing Titers against Influenza A virus (H3N2) from sera of ICR mice immunized intramuscularly (IM) with three different doses of unconjugated composite peptide vaccine (Flu Pep5906+Flu Pep11) with ADDAVAX™ adjuvant.

FIG. 3A Antisera Binding Titers to Influenza A virus (H1N1, H3N2) and Influenza B virus (Yamagata lineage) from sera of ICR mice immunized either intramuscularly (IM) or intradermally (ID) with two different doses of unconjugated composite peptide vaccine (Flu Pep5906+Flu Pep11) with ALFQ adjuvant.

FIG. 3B Antisera Binding Titers to Influenza A virus (H5N1) and Influenza B virus (Yamagata lineage) from sera of ICR mice immunized either intramuscularly (IM) or intradermally (ID) with two different doses of unconjugated composite peptide vaccine (Flu Pep5906+Flu Pep11) with ALFQ adjuvant.

FIG. 4 Antisera Neutralizing Titers against Influenza A virus (H3N2) from sera of ICR mice immunized either intramuscularly (IM) or intradermally (ID) with two different doses of unconjugated composite peptide vaccine (Flu Pep5906+Flu Pep11) with ALFQ adjuvant.

FIG. 5 Binding activity of anti-influenza MABs to various strains of BPL-inactivated Influenza A virus (H5N1, H5N6) and live Influenza B virus (Yamagata lineage). Anti-Flu Pep6 (HA) Mab LD9 at 1, 10, and 25 μg/ml.

FIG. 6 Anti-Flu Pep10 (NA) Mab NB5 at 1, 10, and 25 μg/ml.

FIG. 7 Anti-Flu Pep5906 (M2e) Mab GA4 at 1, 10, and 25 μg/ml.

FIG. 8A Binding of MABs JG7 and GG9 hybridoma supernatant to fixed mycobacteria (strain EK-MTB, Erdman).

FIG. 8B Binding of MABs JG7 and GG9 hybridoma supernatant to fixed mycobacteria (strain HN878).

FIG. 8C Binding of MABs JG7 and GG9 hybridoma supernatant to fixed mycobacteria (strain CDC1551).

FIG. 8D Binding of MABs JG7 and GG9 hybridoma supernatant to fixed mycobacteria (strain M. smegmatis).

FIG. 9 Binding of purified MABs JG7 and GG9 to live mycobacteria.

FIG. 10A Binding of MABs JG7 and GG9 to fixed MTB—Susceptible strain H37Ra.

FIG. 10B Binding of MABs JG7 and GG9 to fixed MTB—multidrug-resistant (MDR).

FIG. 10C Binding of MABs JG7 and GG9 to fixed MTB—extensively drug-resistant (XDR) strain.

FIG. 11 Binding of MABs JG7 and GG9 to various gram-positive bacteria.

FIG. 12A Opsonophagocytic Killing Activity (OPKA) of MABs JG7 and GG9 against Mycobacterium smegmatis (SMEG) using HL-60 granulocytes.

FIG. 12B Opsonophagocytic Killing Activity (OPKA) of MABs JG7 and GG9 against Mycobacterium smegmatis (SMEG) U-937 macrophages.

FIG. 13 OPKA of MAB JG7 against Mycobacterium tuberculosis (MTB) clinical isolate STB1 using U-937 macrophages.

FIG. 14A Rapid clearance of MTB in murine blood by MAB GG9.

FIG. 14B Rapid clearance of MTB in murine blood by MAB JG7.

FIG. 14C Percent mice with undetectable MAB.

FIG. 15 Binding of MABs JG7 and GG9 to Peptidoglycan (PGN).

FIG. 16 Binding profile of antisera from MS 190 immunized with PGN-CRM.

FIG. 17 Binding of anti-PGN antibodies (Day-81 sera) to fixed whole bacteria: staphylococci and mycobacteria.

FIG. 18 OPKA of Anti-PGN antibodies (Day-81 pooled sera from MS 190 group) against SMEG using the macrophage cell line U-937.

FIG. 19 Binding of Anti-PGN Hybridoma MD11 positive clones, in 24-wells, to ultrapure PGN and to various fixed gram-positive bacteria.

FIG. 20 Binding of purified anti-PGN MAB MD11 to ultrapure peptidoglycan from S. aureus and to various fixed whole bacteria.

FIG. 21 Titration of MAB MD11 binding activity to ultrapure PGN and fixed M. smegmatis.

FIG. 22A Competitive inhibition of MAB MD11 binding to ultrapure PGN by small synthetic PGN peptides.

FIG. 22B Competitive inhibition of MAB MD11 binding to the small synthetic PGN peptides by Ultrapure PGN.

FIG. 23 OPKA of anti-PGN MAB MD11 (IgG2) against M. smegmatis using U-937 macrophages and HL60 granulocytes.

FIG. 24 OPKA of anti-PGN MABs JG7, GG9 (IgG1) and MD11 (IgG2) against Staphylococcus epidermidis using Polymorphonuclear cells (PMNs).

FIG. 25A Binding of MAB JG7 to PGN peptides, PGN Pep1-Pep6.

FIG. 25B Binding of MAB GG9 to PGN peptides, PGN Pep1-Pep6.

FIG. 25C Binding of MAB MD11 to PGN peptides, PGN Pep1-Pep6.

FIG. 26 Binding of MABs JG7, GG9 and MD11 to Ultrapure PGN from S. aureus.

FIG. 27 Binding of MABs LD7 and CA6 hybridoma supernatant to alpha crystallin HSP.

FIG. 28 Binding of purified MABs LD7 and CA6 to live mycobacteria (SMEG) in mid-log phase, live, heat-treated or ethanol-treated SMEG.

FIG. 29 Binding of MABs LD7 and CA6 (purified from subclones) to live mycobacteria.

FIG. 30 Opsonophagocytic Killing Activity (OPKA) of MABs LD7 and CA6 against Mycobacterium smegmatis (SMEG) using U-937 macrophages.

FIG. 31A Serum antibody responses in mice immunized subcutaneously with 20 μg dose of Coronavirus Pep02. IgG1 antisera titers to immunogens.

FIG. 31B Serum antibody responses in mice immunized subcutaneously with 20 μg dose of Coronavirus Pep05. IgG1 antisera titers to immunogens.

FIG. 31C Serum antibody responses in mice immunized subcutaneously with 20 μg dose of Coronavirus Pep11. IgG1 antisera titers to immunogens.

FIG. 32A Serum antibody responses in mice immunized subcutaneously with 20 μg dose of Coronavirus Pep02. IgG2b antisera titers to immunogens.

FIG. 32B Serum antibody responses in mice immunized subcutaneously with 20 μg dose of Coronavirus Pep05. IgG2b antisera titers to immunogens.

FIG. 32C Serum antibody responses in mice immunized subcutaneously with 20 μg dose of Coronavirus Pep11. IgG2b antisera titers to immunogens.

FIG. 33A Serum antibody responses in mice immunized subcutaneously with 20 μg dose of Coronavirus Pep02. IgG1 antisera titers to immunogens.

FIG. 33B Serum antibody responses in mice immunized subcutaneously with 20 μg dose of Coronavirus Pep05. IgG1 antisera titers to immunogens.

FIG. 33C Serum antibody responses in mice immunized subcutaneously with 20 μg dose of Coronavirus Pep02. IgG2b antisera titers to immunogens.

FIG. 33D Serum antibody responses in mice immunized subcutaneously with 20 μg dose of Coronavirus Pep05. IgG2b antisera titers to immunogens.

FIG. 34A Serum antibody responses in mice immunized with Coronavirus Pep05. Profile of IgG1 antisera titers to the composite coronavirus peptides.

FIG. 34B Serum antibody responses in mice immunized with Coronavirus Pep05. Profile of IgG1 antisera titers to influenza epitopes.

FIG. 34C Serum antibody responses in mice immunized with Coronavirus Pep05. Profile of IgG1 antisera titers to individual coronavirus RNA polymerase and spike protein epitopes.

FIG. 34D Serum antibody responses in mice immunized with Coronavirus Pep11. Profile of IgG1 antisera titers to the composite coronavirus peptides.

FIG. 34E Serum antibody responses in mice immunized with Coronavirus Pep11. Profile of IgG1 antisera titers to influenza.

FIG. 34F Serum antibody responses in mice immunized with Coronavirus Pep11. Profile of IgG1 antisera titers to individual coronavirus RNA polymerase and spike protein.

FIG. 35A Serum antibody responses in mice immunized with Coronavirus Pep05. Profile of IgG2b antisera titers to the composite coronavirus peptides.

FIG. 35B Serum antibody responses in mice immunized with Coronavirus Pep05. Profile of IgG2b antisera titers to influenza epitopes.

FIG. 35C Serum antibody responses in mice immunized with Coronavirus Pep05. Profile of IgG2b antisera titers to individual coronavirus RNA polymerase and spike protein.

FIG. 35D Serum antibody responses in mice immunized with Coronavirus Pep11. Profile of IgG2b antisera titers to the composite coronavirus peptides.

FIG. 35E Serum antibody responses in mice immunized with Coronavirus Pep11. Profile of IgG2b antisera titers to influenza epitopes.

FIG. 35F Serum antibody responses in mice immunized with Coronavirus Pep11. Profile of IgG2b antisera titers to individual coronavirus RNA polymerase and spike protein epitopes.

FIG. 36A Serum antibody responses in select mice immunized subcutaneously with 20 μg dose of either Coronavirus Pep11 or Coronavirus Pep05. One year post primary immunizations, the selected mice were given a boost and bled a week after. IgG1 antibody titers to coronavirus peptides.

FIG. 36B Serum antibody responses in select mice immunized subcutaneously with 20 μg dose of either Coronavirus Pep11 or Coronavirus Pep05. One year post primary immunizations, the selected mice were given a boost and bled a week after. IgG1 antibody titers to influenza epitopes.

FIG. 37A Serum antibody responses in select mice immunized subcutaneously with 20 μg dose of either Coronavirus Pep11 or Coronavirus Pep05. One year post primary immunizations, the selected mice were given a boost and bled a week after. IgG antibody titers to influenza virus A.

FIG. 37B Serum antibody responses in select mice immunized subcutaneously with 20 μg dose of either Coronavirus Pep11 or Coronavirus Pep05. One year post primary immunizations, the selected mice were given a boost and bled a week after. IgG antibody titers to human Coronavirus.

FIG. 38 Neutralizing titers in select mice immunized subcutaneously with 20 μg dose of either Coronavirus Pep11 or Coronavirus Pep05.

FIG. 39A IgG1 antisera titers (day 70) to various subtypes of influenza virus in mice immunized with Coronavirus Pep05 and Coronavirus Pep11.

FIG. 39B Neutralizing titers to influenza virus of Group1 (H1N1) and Group2 (H3N2) subtypes.

FIG. 40A IgG1 antisera titers (day 266) to human Coronavirus (hCoV) NL-63 are demonstrated.

FIG. 40B End-point neutralization titers based on 75% neutralization of hCoV NL-63 are shown as PRNT75 values.

FIG. 41 IgG1 antisera titers (day 252) to three variants of gamma-irradiated SARS CoV-2 are shown in mice immunized with Coronavirus Pep02, Coronavirus Pep05 and Coronavirus Pep11.

FIG. 42A Serum antibody responses in mice immunized with a combination of Coronavirus Pep05 and Coronavirus Pep11. Profile of IgG1 antisera titers to the composite coronavirus peptides and influenza epitopes.

FIG. 42B Serum antibody responses in mice immunized with a combination of Coronavirus Pep05 and Coronavirus Pep11. Profile of IgG2b antisera titers to the composite coronavirus peptides and influenza epitopes.

FIG. 42C Serum antibody responses in mice immunized with a combination of Coronavirus Pep05 and Coronavirus Pep11. Day 21 (pre-boost) virus binding titers (IgG1) to various subtypes of influenza A and B and three variants of SARS-CoV-2.

FIG. 43A Serum antibody responses in mice immunized intradermally with 1 μg dose of a composite vaccine comprising of Coronavirus Pep11 and Coronavirus Pep05 and booster immunizations given on days 21 and 35. IgG1 antisera titers to the coronavirus peptides for each dose group.

FIG. 43B Serum antibody responses in mice immunized intradermally with 10 μg dose of a composite vaccine comprising of Coronavirus Pep11 and Coronavirus Pep05 and booster immunizations given on days 21 and 35. IgG1 antisera titers to the coronavirus peptides for each dose group.

FIG. 43C Serum antibody responses in mice immunized intradermally with 20 μg dose of a composite vaccine comprising of Coronavirus Pep11 and Coronavirus Pep05 and booster immunizations given on days 21 and 35. IgG1 antisera titers to the coronavirus peptides for each dose group.

FIG. 43D Serum antibody responses in mice immunized intradermally with 1 μg dose of a composite vaccine comprising of Coronavirus Pep11 and Coronavirus Pep05 and booster immunizations given on days 21 and 35. IgG1 antisera titers to influenza epitopes and universal T cell epitopes.

FIG. 43E Serum antibody responses in mice immunized intradermally with 10 μg dose of a composite vaccine comprising of Coronavirus Pep11 and Coronavirus Pep05 and booster immunizations given on days 21 and 35. IgG1 antisera titers to influenza epitopes and universal T cell epitopes for each dose group.

FIG. 43F Serum antibody responses in mice immunized intradermally with 20 μg dose of a composite vaccine comprising of Coronavirus Pep11 and Coronavirus Pep05 and booster immunizations given on days 21 and 35. IgG1 antisera titers to influenza epitopes and universal T cell epitopes for each dose group.

FIG. 44A Serum antibody responses in mice immunized intradermally with 1 μg, 10 μg or 20 μg dose of a composite vaccine comprising of Coronavirus Pep11 and Coronavirus Pep05. One year post primary immunizations, the mice were given a boost and bled a week after. IgG antibody titers to influenza virus A.

FIG. 44B Serum antibody responses in mice immunized intradermally with 1 μg, 10 μg or 20 μg dose of a composite vaccine comprising of Coronavirus Pep11 and Coronavirus Pep05. One year post primary immunizations, the mice were given a boost and bled a week after. IgG antibody titers to human Coronavirus.

FIG. 45 Neutralizing titers in mice immunized intradermally with 1 μg, 10 μg or 20 μg dose of a composite vaccine comprising of Coronavirus Pep11 and Coronavirus Pep05.

FIG. 46A Serum antibody responses in select mice immunized intradermally with 10 μg of a composite vaccine comprising of Coronavirus Pep11 and Coronavirus Pep05. One year post primary immunizations, the mice were given a boost and bled a week after. IgG1 antibody titers to coronavirus peptides.

FIG. 46B Serum antibody responses in select mice immunized intradermally with 10 μg of a composite vaccine comprising of Coronavirus Pep11 and Coronavirus Pep05. One year post primary immunizations, the mice were given a boost and bled a week after. IgG1 antibody titers to influenza epitopes.

FIG. 47A Serum antibody responses in select mice immunized intradermally with 10 μg of a composite vaccine comprising of Coronavirus Pep11 and Coronavirus Pep05. One year post primary immunizations, the mice were given a boost and bled a week after. IgG titers to influenza virus A.

FIG. 47B Serum antibody responses in select mice immunized intradermally with 10 μg of a composite vaccine comprising of Coronavirus Pep11 and Coronavirus Pep05. One year post primary immunizations, the mice were given a boost and bled a week after. IgG titers to human Coronavirus.

FIG. 48 Neutralizing titers in select mice immunized intradermally with 10 μg of a composite vaccine comprising of Coronavirus Pep11 and Coronavirus Pep05.

FIG. 49A Profile of serum antibody responses to PGN and TB Pep 01 analyzed using IgM detection antibodies.

FIG. 49B Profile of serum antibody responses to PGN and TB Pep 01 analyzed using IgG detection antibodies.

FIG. 49C Day-42 serum antibody responses to MTB CDC1551.

FIG. 50A Human IgM Hybridoma Clones DRG-5 BD11 supernatant binding activity to PGN and TB Pep 01.

FIG. 50B Human IgM mAb DRG-5 BD11 IIG1 binding activity to PGN and various live gram-positive bacteria at 10\hat{ }5 CFU/mL.

FIG. 51A Binding activity of antisera from pigs immunized with LHNVD-105 at 100 μg, 250 μg, or 500 μg dose or PBS (phosphate buffered saline) on d0 and d28 to Flu A/California (H1N1)pdm09. Data are represented as means±SEM.

FIG. 51B Binding activity of antisera from pigs immunized with LHNVD-105 at 100 μg, 250 μg, or 500 μg dose or PBS on d0 and d28 to Flu A/Hong Kong/4801/2014 (H3N2). Data are represented as means±SEM. FIG. 51C Binding activity of antisera from pigs immunized with LHNVD-105 at 100 μg, 250 μg, or 500 μg dose or PBS on d0 and d28 to LHNVD-105. Data are represented as means±SEM.

FIG. 51D HAI titers of d49 antisera from pigs immunized with LHNVD-105 at 100 μg, 250 μg, or 500 μg dose or PBS against Flu A/California/2009 (H1N1)pdm09. Data are represented as Geometric Mean Titer (GMT) with standard error.

FIG. 51E HAI titers of d49 antisera from pigs immunized with LHNVD-105 at 100 μg, 250 μg, or 500 μg dose or PBS against Flu A/Hong Kong/4801/2014 (H3N2). Data are represented as Geometric Mean Titer (GMT) with standard error.

### DESCRIPTION OF THE INVENTION

Vaccinations and vaccines are often the best mechanism for avoiding an infection and preventing the spread of debilitating and dangerous pathogens. With respect to viral infections, parasitic infections and many bacterial infections, vaccinations may be the only effective option as preventative or treatment options are few and those that are available provide only limited effectiveness. Conventional vaccinations require a priori understanding or general identification of the existing antigenic regions of the pathogen. The pathogen itself is propagated and a suitable vaccine developed from heat-killed or otherwise attenuated microorganisms. Alternatively, an antigen or collection of antigens is identified that will generate a protective immune response upon administration. The need for a vaccine is especially urgent with respect to preventing infection by certain bacteria, viruses and parasites. Some bacteria and especially certain viruses mutate constantly or mutate when passing through an intermediate host, often rendering the vaccine developed to the prior or originating bacteria or virus useless against the new strains that emerge. As a consequence, some vaccines may need to be reformulated yearly (or more often) and often administered at fairly high doses. The development and manufacturing costs are high and administering vaccines pose a great many complications and associated risks to patients.

Antigens and epitopes as disclosed herein, including specific combination as described herein, were surprisingly discovered. These antigens contain or are derived from a plurality of antigenic regions (e.g., epitopes which may be continuous or discontinuous epitopes) of a pathogen or of different pathogens. Most all viruses such as Influenza virus and Corona virus, and most all bacterial microbes contain both continuous and discontinuous epitopes. For example, the various strains of Influenza virus (e.g., H1N1; H1N9; H3N2; H3N8; H5N2; H7N7; H9N2) contain hundreds of epitopes. Epitopes A198, S199, R201 (H3N2) of influenza virus HA protein are believed to be continuous, whereas other epitopes of HA protein (e.g., G49, K50, L59, D60, 162, D63, P74, H75, V78, F79, R90, K92, F94, P143, D271, P273, 1274, D27; H3N2) are believed to be discontinuous. Epitopes D147, H150, H197, D198, E199, K221, D251 (H3N2) of influenza virus NA proteins are believed to be discontinuous, whereas epitopes S367, S372, N400 (H1N9) are believed to be continuous.

Composite antigens of the invention may contain an antigenic region that represents a combination of all or parts of two or more epitopes (e.g., a composite peptide), or a plurality of immunologically responsive regions derived from one or multiple antigenic sources (e.g., epitopes of viruses, parasites, bacteria, fungi, cells). These immunological regions are amino acid sequences or epitopes that are generally highly conserved sequences found at those antigenic regions of a pathogen or other antigen associated with an infection or a disease or, importantly, associated with stimulation of the immune system to provide protection against the pathogen. Vaccines may be administered via injection (e.g., intramuscular, intradermal, intravenous, intraperitoneal) or taken orally or intranasally. Preferably, immunogenic compositions are administered collectively to animals such as in a water or food supply, or as an aerosol dispensed in a closed or partially closed environment, thereby avoiding the need and expense of providing the vaccine individually.

Composite epitope vaccine antigen sequences are unique peptide antigens that combine conserved peptide sequences from the same, or different microbes into one sequence that provides a peptide that is different from any peptide sequence found in nature. Peptide epitopes may be known or previously unknown epitopes that have been identified in microbes such as bacteria, parasites, fungi, or viruses. One or more epitopes from a single microbe can be sequenced as a single, or repeated epitope and may be combined with one or more epitopes from one or more other pathogens in a continuous peptide sequence. The composite peptide antigens may be to a single microbe or to one or more microbes, or viruses, such as for example, influenza, coronavirus, adenovirus, and respiratory syncytial virus. The composite peptide antigen may also be from a single bacterium, or from one or more gram positive, or gram-negative bacteria, such Pneumococcus spp., Staphylococcus spp. (e.g., S. aureus), Mycobacteria spp. (e.g., M. tuberculosis, M. smegmatis, M. leprae, M. kansasii, M. mantenii, M. fortuitum, or M. xenopi), Bacillus spp. (e.g., B. subtilis), Escherichia spp. (e.g., E. coli), Haemophilus spp. (e.g., H. influenza), Salmonella spp., etc. The epitopes may be combined in any order or configured to provide an immunogenic structure that induces an immune response in a host immunized with the composite peptide vaccine.

One embodiment of the invention is directed to peptide epitopes of a pathogen, such as viral, parasitic and/or bacterial antigens. Antigens and peptide epitopes disclosed herein may be selected regions of a viral, parasitic, and/or bacterial microbe that is known or believed to generate an effective immune response after administration. The peptide composite (composite antigen) sequence may contain a plurality of immunologically responsive regions or epitopes of one or more pathogens, which are artificially arranged, preferably along a single amino acid sequence or peptide. The plurality may contain multiples of the same epitope, although generally not in a naturally occurring order, or multiples of a variety of different epitopes from one or more pathogens. Epitopes may be identical to known immunological regions of a pathogen, or entirely new constructs that have not previously existed and therefore artificially constructed. Preferably, the composite antigen of this disclosure induces a protective immunogenic response in the animal or a mammal (e.g., human) and stimulates both mucosal and systemic immune responses similar to those of the natural infection. Preferably that response includes the production of killer T-cell (T_{C} or CTL) responses, helper T-cell (T_{H}) responses, macrophages (MP), and specific antibody production in an inoculated subject. Also preferably the response generated is opsonic.

Composite antigens of the invention may also be obtained or derived from the sequences of a pathogen such as, for example, multiple or combined epitopes of the proteins and/or polypeptides of gram-positive and/or gram-negative bacteria, for example, but not limited to Streptococcus, Pseudomonas, Mycobacterium such as M. tuberculosis, Shigella, Campylobacter, Salmonella, Haemophilus influenza, Chlamydophila pneumonia, Corynebacterium diphtheriae, Clostridium tetani, Mycoplasma pneumonia, Staphylococcus aureus, Moraxella catarrhalis, Legionella pneumophila, Bordetella pertussis, Escherichia coli, such as E. coli 0157, and multiple or combined epitomes of conserved regions of any of the foregoing. Exemplary parasites from which sequences may be obtained or derived include but are not limited to Plasmodium such as Plasmodium falciparum and Trypanosoma. Exemplary fungi include, but are not limited to, Aspergillus fumigatus and Aspergillus flavus. Exemplary viruses include, but are not limited to arena viruses, bunyaviruses, coronaviruses, paramyxoviruses, filoviruses, Hepadna viruses, herpes viruses, orthomyxoviruses, orthopneumovirus, parvoviruses, picornaviruses, papillomaviruses, reoviruses, retroviruses, rhabdoviruses, and togaviruses. Preferably, the virus epitopes are obtained or derived from sequences of Influenza viruses.

Antigens as disclosed herein include composite antigens, which are engineered, artificially created antigens made from two or more epitopes, such that the resulting composite antigen has physical and/or chemical properties that differ from or are additive of the individual epitopes. Preferable the composite antigen, when exposed to the immune system of a mammal or an animal, is capable of simultaneously generating an immunological response to each of the constituent epitope of the composite and preferably to a greater degree (e.g., as measurable from a cellular or humoral response to an identified pathogen) than the individual epitopes. In addition, the composite antigen provides the added function of generating a protective immunological response in a mammal or an animal when used as a vaccine and against each of the constituent epitopes. Preferably, the composite has the additional function of providing protection against not only the pathogens from which the constituents were derived, but related pathogens as well. These related pathogenic organisms may be different strains and/or different serotypes of the same species of organism, or different species of the same genus of organism, or different organisms entirely that are only related by a common epitope.

Composite peptides may contain one or more composite epitopes that represent two or more epitopes with epitope sequences only similar to the epitope sequences from which they were derived. Epitopes are regions obtained or derived from a conserved region of a protein or peptide of a pathogen that elicit a robust immunological response when administered to a mammal or an animal. Preferably, that robust response provides the subject with an immunological protection against developing disease from exposure to the pathogen. A preferred example is a composite epitope, which is one artificially created from a combination of two or more highly conserved, although not identical, amino acid sequences of two or more different, but otherwise related pathogens. The pathogens may be of the same type, but of a different strain, serotype, or species or other relation. The composite epitope contains the conserved region that is in common between the related epitopes, and also contains the variable regions which differ. The sequences of a composite epitope that represents a combination of two conserved, but not identical sequences, may be illustrated as follows:

| | |
|---|---|
| Sequence of Epitope 1 | . . . AAAAABAAAAA . . . |
| Sequence of Epitope 2 | . . . AAAAACAAAAA . . . |
| Composite Epitope | . . . AAAAABCAAAAA . . . |

wherein, “A” represents the amino acids in common between the two highly conserved epitopes, “B” and “C” represent the amino acids that differ, respectively, between two epitopes, each of “A”, “B” and “C” can be any amino acid and any number of amino acids. Preferably the conserved region contains about 20 or less amino acids on each side of the variable amino acids, preferably about 15 or less, preferably about 10 or less, preferably about 8 or less, preferably about 6 or less, and more preferably about 4 or less. Preferably the amino acids that vary between two similar, but not identical conserved regions are 5 or less, preferably 4 or less, preferably 3 or less, preferably 2 or less, and more preferably only 1.

A “composite epitope,” similar to the composite antigen, is an engineered, artificially created single epitope made from two or more constituent epitopes, such that the resulting composite epitope has physical and/or chemical properties that differ from or are additive of the constituent epitopes. Preferable the composite epitope, when exposed to the immune system of a mammal or an animal, is capable of simultaneously generating an immunological response to each of the constituent epitopes of the composite and preferably to a greater degree than that achieved by either of the constituent epitopes individually. In addition, the composite epitope provides the added function of generating a protective immunological response in a patient when used as a vaccine and against each of the constituent epitopes. Preferably, the composite has the additional function of providing protection against not only the pathogens from which the constituents were derived, but related pathogens as well. These related pathogenic organisms may be strains or serotypes of the same species of organism, or different species of the same genus of organism, or different organisms entirely that are only related by a common epitope.

Composite epitopes of the invention are entirely artificial molecules that do not otherwise exist in nature and to which an immune system has not been otherwise exposed. Preferably, these conserved immunological regions that are combined as a composite epitope represent immunologically responsive regions of proteins and/or polypeptides that are highly conserved between related pathogens. Although a vaccine can be developed from a single composite epitope, in many instances the most effective vaccine may be developed from multiple, different composite epitopes.

Composite antigens of the invention may contain one or more epitopes or composite epitopes, which may include one or more known epitopes to provide an effective vaccine. Although composite antigens may comprise a single composite epitope, a composite antigen would not comprise only a single known epitope. Preferably, the immunological response achieved from a vaccination with a composite antigen, or group of composite antigens, provides protection against infection caused by the original strains from which the sequence of the composite antigen was derived and also provides immunological protection against other strains, serotypes and/or species that share one or more of the general conserved regions represented in the composite antigen. Preferably that response stimulates both mucosal and systemic immune responses in the mammal or the animal, similar to those of the natural infection. Thus, the resulting immune response achieved from a vaccination with a composite antigen is more broadly protective than can be achieved from a conventional single antigen vaccination against multiple strains, serotypes, and species or otherwise related pathogens regardless of antigenic drift that may take place in the evolution of the pathogen. Preferably, vaccines developed from composite antigens of the invention avoid any need for repeated or annual vaccinations, the associated complications and expenses of manufacture, and the elevated risks to the subject. These vaccines are useful to treat individual animals, mammals, and populations or either, thereby preventing infection and mortality and subsequently infections in mammals including pandemics. Such vaccines are also useful to compliment conventional vaccines.

As discussed herein, the composite antigen preferably comprises a single chain of amino acids with a sequence derived from one or more epitopes or a plurality of epitopes, that may be the same or different. Epitope sequences may be repeated consecutively and uninterrupted along a composite sequence or interspersed among other sequences that may be single or a few amino acids as spacers or sequences that encode peptides (collectively spacers), and may be nonimmunogenic or immunogenic and capable of inducing a cellular (T cell) or humoral (B cell) immune response in an animal or a mammal. T-cell stimulating antigens include, for example, tetanus toxin, tetanus toxin heavy chain proteins, diphtheria toxoid (e.g., recombinantly engineered or purified CRM197), tetanus toxoid, Pseudomonas exoprotein A, Pseudomonas aeruginosa toxoid, Bordetella pertussis toxoid, Clostridium perfringens toxoid, Escherichia coli heat-labile toxin B subunit, Neisseria meningitidis outer membrane complex, Hemophilus influenzae protein D, Flagellin Fli C, Horseshoe crab Haemocyanin, and fragments, derivatives, and modifications thereof. Peptides sequence from unrelated microbes may be combined into a single composite antigen. For example, viral sequences of selected immunoresponsive peptides may be interspersed with conserved sequences or epitopes selected from other microbes, such as, for example, bacteria such as M. tuberculosis, S. pneumococcus, P. aeruginosa or S. aureus, viruses such as respiratory viruses, or parasites, such as malaria. Preferred viral proteins, from which preferred epitopes may be selected, include, but are not limited to the influenza virus proteins HA, NA, and M2e, and/or coronavirus proteins spike (S), polymerase (POL), envelope (E), membrane (M), and nucleocapsid (N).

An epitope of the composite antigen may be of any sequence and size, but is preferable composed of natural amino acids or mimotopes (i.e., a peptide and mimics the structure of an epitope but is composed of a different amino acid sequence than the natural epitope) and is more than 5 but less than 100 amino acids in length, preferably less than 80, preferably less than 70, preferably less than 60, preferably less than 50, preferably less than 40, preferably less than 30, preferably between 5 and 25 amino acids in length, preferably between 8 and 20 amino acids in length, and more preferably between 5 and 15 amino acids in length. Mimotopes may have 1, 2, 3, 4, 5, 6, 7, 8, 9, 10 or more amino acid differences as compared to the natural epitope. Composite antigens preferably contain any number of composite and/or other epitopes. The most effective number of epitopes of a composite antigen against a particular pathogen, pathogen family, or group of pathogens may be determined by one skilled in the art from the disclosures of this application and using routine testing procedures. Composite antigens may be effective with one epitope, preferably with 2 or more, 3 or more 4 or more, 5 or more or greater. Optionally, composite antigens may include one or more spacers between epitopes which may be sequences of antigenic regions derived from the same or from one or more different pathogens, or sequences that serve as immunological primers or that otherwise provide a boost to the immune system. That boost may be generated from a sequence of amino acids that are known to stimulate the immune system, either directly or as an adjuvant. Preferred adjuvants comprise analgesic adjuvants, inorganic compounds such as alum, aluminum hydroxide, oil in water emulsion, squalene oil in water nano-emulsion, aluminum phosphate, calcium phosphate hydroxide, mineral oil such as paraffin oil, bacterial products such as killed bacteria Bordetella pertussis, Mycobacterium bovis, toxoids, nonbacterial organics such as squalene, detergents, plant saponins such as Quillaja (Quil A), soybean, Polygala senega, cytokines such as IL-1, IL-2, IL-12, Freund's complete adjuvant, Freund's incomplete adjuvant, food-based oil, Adjuvant 65, which is a product based on peanut oil, and derivatives, modifications and combinations thereof. Preferred adjuvants include, for example, AS01 (Adjuvant System 01) which comprises TLR4 ligand, 3-O-desacyl-4′-monophosphoryl lipid (MPL), and a saponin, QS-21, AS01b which is a component of the adjuvant Shingrix, ALF (Army Liposome Formulation) which comprises liposomes containing saturated phospholipids, cholesterol, and/or monophosphoryl lipid A (MPLA) as an immunostimulant. ALF has a safety and a strong potency. ALF modifications and derivatives include, for example, ALF adsorbed to aluminum hydroxide (ALFA), ALF containing QS21 saponin (ALFQ), and ALFQ adsorbed to aluminum hydroxide (ALFQA). A preferred adjuvant formulation comprises a liposome, saponin, lipid A, squalene, unilamellar liposomes having a liposome bilayer that comprises at least one phosphatidylcholine (PC) and/or phosphatidylglycerol (PG), as phospholipids, which may be dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), and/or distearyl phosphatidylglycerol (DSPG), a cholesterol, a monophosphoryl lipid A (MPLA), and a saponin. Preferably the mole ratio of the cholesterol to the phospholipids is greater than about 50:50, and also that the unilamellar liposomes have a median diameter size in micrometer range as detected by light scattering analysis. Additional preferred adjuvants are disclosed in U.S. Pat. No. 10,434,167, which issued Oct. 8, 2019, the entirety of which is incorporated by reference herein.

In one preferred form, composite antigens useful to generate an immunological response against influenza virus comprise epitopes of HA, M or Matrix (M1, M2, M2e), and/or NA proteins, and/or new epitopes derived from similar conserved regions of different serotypes and strains of influenza virus, and/or from the S or POL protein of coronavirus. Also preferred are composite antigens containing epitopes of proteins of Mycobacterium tuberculosis and Clostridium tetani, and/or new epitopes derived from similar conserved regions of different serotypes of these bacteria. Another preferred composite antigen would include HIV and or malaria epitopes combined with one or more of the above microbial epitopes.

Another form of the antigen comprises a contiguous sequence of one or more epitopes, which may comprise composite and/or known epitopes, from one or more pathogens in a sequence that does not exist naturally and must be artificially constructed. For example, a contiguous sequence may contain epitopes in closer proximity to each other than would otherwise occur naturally or may contain spacer sequences between epitopes that do not otherwise occur naturally. Preferably, a contiguous sequence of the invention contains one or more composite epitopes, which is a combination of the sequences of the conserved regions of epitopes that are common to multiple pathogens plus those amino acids that differ between the two conserved regions. For example, where two pathogens contain similar conserved regions that differ by only a single amino acid, the composite sequences would include the conserved region amino acids and each of the amino acids that differ between the two regions as discussed herein.

It is also preferable that a composite antigen of the invention contain a plurality of repeated epitopes and, optionally, epitopes conjugated with linker regions between or surrounding each epitope, and the plurality of epitopes be the same or different. Preferred linkers include amino acid sequences of antigenic regions of the same or of different pathogens, or amino acids sequences that aid in the generation of an immune response. Preferred examples include, but are not limited to, any of the various antigenic regions of bacteria such as, but not limited to M. tuberculosis, S. aureus and E. coli and viruses such as, but not limited to influenza, coronavirus and HIV and parasites such as P. falciparum. It is also preferred that composite antigens contain epitopes that generate a systemic and/or a mucosal immune responses similar to that produced from a natural infection.

Another embodiment of the invention is directed to methods for treating or preventing infection of bacteria, virus, parasites, or other microorganisms in a mammal comprising administering to the mammal polyclonal or monoclonal antibodies that are specifically reactive against the peptides disclosed here. Preferably the polyclonal or monoclonal antibodies generate cellular phagocytic activity, destruction of the microorganism, enhances cytokine induced immunity to the microorganism or neutralizes toxic substances of the microorganism, and/or cocktails of two or more monoclonal antibodies (MABs) that enhance immunity to the microorganism. Preferably, the anti-microorganism antibodies are polyclonal antibodies or monoclonal antibodies and react against one or more MTB moieties.

Another embodiment of the invention is directed to monoclonal antibodies that are specifically reactive against epitopes of the microorganism. Preferably the monoclonal antibody is an IgA, IgD, IgE, IgG or IgM (including subtypes thereof such as, for example, IgG1, IgG2, IgG2A, IgG2B, IgG2C, IgG3 and IgG4), and may be derived from most any mammal such as, for example, human, porcine, caprine, murine, leporidae, muridae, and equine, to include rabbit, guinea pig, mouse, human, fully or partly humanized, chimeric or single chain of any of the above. The DNA encoding the antibodies may be utilized in any appropriate cell line to produce the encoded MABs. Another embodiment comprises hybridoma cultures that produce the monoclonal antibodies. Another embodiment of the invention comprises non-naturally occurring polyclonal antibodies that are specifically reactive against the microorganism. Some important monoclonal antibodies are described in Table 1.

TABLE 1

| | | | Hybridoma | Hybridoma | Target | mAb |
|---|---|---|---|---|---|---|
| Mouse ID | Immunogen | Conjugate | (mAb) ID | Clone ID | Antigen | Isotype |
| MS 1435 | TB Pep01 | CRM | LD7 | LD7 I BB2 I | 16 kD HSP16.3 | IgG2a |
| | | | | B9 | TB Pep01 | |
| MS 1435 | TB Pep01 | CRM | CA6 | CA6 II GA8 I | 16 kD HSP16.3 | IgG2b |
| | | | | A5 | TB Pep01 | |
| MS 190 | Ultrapure | CRM | MD11 | MD11 I C11 | PGN | IgG2b |
| | Peptidoglycan from | | | | | |
| | S. aureus (PGN) | | | | | |
| MS 2209 | A/Wuhan (H3N2) + | CRM | NB5 | NB5 II C2 I K8 | Neuraminidase | IgG2a |
| | Flu Pep11 | | | | (NA) Flu | |
| | | | | | Pep10 | |
| MS 2209 | A/Wuhan (H3N2) + | CRM | LD9 | LD9 III D6 | Hemagglutinin | IgG1 |
| | Flu Pep11 | | | | (HA) Flu | |
| | | | | | Pep06 | |
| MS 2209 | A/Wuhan (H3N2) + | CRM | EA9 | EA9 I F7 | Hemagglutinin | IgG1 |
| | Flu Pep11 | | | | (HA) Flu | |
| | | | | | Pep03 | |
| MS 1443 | Flu Pep5906 | CRM | GA4 | GA4 I G11 | Matrix | IgG1 |
| | | | | | (M1/M2/M2e) | |
| | | | | | Flu Pep5906 | |
| MS 2016 | Flu Pep5906 + | CRM | CG6 | CG6 II H8 | Matrix | IgG3 |
| | Flu Pep11 | | | | (M1/M2/M2e) | |
| | | | | | Flu Pep5906 | |
| MS 2016 | Flu Pep5906 + | CRM | KC7 | KC7 I D8 | Matrix | IgG3 |
| | Flu Pep11 | | | | (M1/M2/M2e) | |
| | | | | | Flu Pep5906 | |
| DRAGA | Ultrapure | CRM | DRG-5 BD11 | DRG-5 BD11 | PGN | IgM |
| 5 | Peptidoglycan from | | | II E6 II G1 | | |
| | S. aureus (PGN) + | | | | | |
| | TB Pep01 | | | | | |

Hybridoma cell lines that express the monoclonal antibodies disclosed herein were deposited with the American Type Culture Collection (ATCC; Manassas, VA). Hybridomas that produce monoclonal antibodies EA9 (PTA-127659), KC7 (PTA-127660), DRG-5BD11(PTA-127658), CG6 (PTA-127661), and LD9 (PTA-127662) (as identified in Table 1) were each deposited with ATCC on Oct. 13, 2023. Monoclonal antibodies produced by these hybridomas may include variable and hypervariable regions, CDR, and Fc regions that may be separately obtained and useful as such. These monoclonal antibodies may be fully or partly humanized, bispecific, and/or conjugated. Another embodiment of the invention is directed to methods for treating or preventing infection by administering a monoclonal or polyclonal antibody that is specifically reactive against the microorganism.

Another embodiment of the invention is directed to method of immunizing mammals or animals with the immunogenic compositions of the invention. Preferably, the vaccines of the invention are less susceptible to variation of antigenicity due to antigenic shift of pathogens which reduces or eliminates the need for annual or repeated vaccination to maintain protection of the mammal or animal populations against potential outbreaks of infection from, for example, new bacterial strain or viral isolates. In addition, the vaccines of the invention generally and advantageously provide increased safety considerations, both in their manufacture and administration (due in part to a substantially decreased need for repeated administration), a relatively long shelf life in part due to minimized need to reformulate due to strain-specific shift and drift, an ability to target immune responses with high specificity for particular microbial epitopes, and an ability to prepare a single vaccine that is effective against multiple pathogens, each of which may be a different. As single immunization to provided protection against one, or more viruses, bacteria, or parasites, such as influenza, coronavirus, HIV, M. tuberculosis, S. aureus, or malaria. The invention encompasses antigenic compositions, methods of making such compositions, and methods for their use in the prevention, treatment, management, and/or prophylaxis of an infection. The compositions disclosed herein, as well as methods employing them, find particular use in the treatment or prevention of viral, bacterial, parasitic and/or fungal pathogenesis and infection using immunogenic compositions and methods superior to conventional treatments presently available in the art. Preferably, vaccinations of immunogenic compositions of antigens disclosed herein provide protection against a pathogenic infection for more than a one-year cycle, which is typical for pathogens such as influenza virus. More preferably, protection is provided for up to 2 years, 5 years, 10 years, 15 years, 20 years, or longer.

These methods can prevent or control infections, such as, for example, an outbreak of viral, parasitic, fungal or bacterial infection, preferably but not limited to an influenza virus, coronavirus, and/or a tuberculosis bacterial infection, in a selected population of animals or mammals. The method includes at least the step of providing an immunologically effective amount of one or more of the disclosed immunogenic or vaccine compositions to a susceptible or an at-risk animal of a population, for a time sufficient to prevent, reduce, lessen, alleviate, control, or delay the outbreak of such an infection in the general population. Preferably, the administration is performed into the water or food supply, or as an aerosol into a closed or semi-closed environment where the animals are maintained, even temporarily maintained.

Another embodiment of the invention is directed to an immunogenic composition comprising nucleic acid sequences that encode protective antigens and/or epitopes against a pathogen. The sequences can be incorporated into a viral vector, suitable for immunizing a mammal. Preferred pathogens include, but are not limited to bacteria, viruses, parasites, fungi and viruses.

In a preferred example, antigens contain a conserved region derived from an influenza virus subtypes (e.g., influenza viruses with varying HA or NA compositions, such as H1N1, H5N1, H3N2, and H2N2). Epitopes of conserved regions on NA or HA may also confer cross-subtype immunity. As an example, conserved epitopes on NA(N1) may confer enhanced immunity to H5N1 and H1N1. With respect to similar or homologous chemical compounds among influenza A subtypes and/or strains within a subtype, preferably these are at least about 80 percent, more preferably at least about 90 percent, more preferably at least about 95 percent identical, more preferably at least about 96 percent identical, more preferably at least about 97 percent identical, more preferably at least about 98 percent identical, more preferably at least about 99 percent identical, and even more preferably 100 percent identical (invariant). Preferably, at least one peptide sequence within the composite antigen is also conserved on homologous proteins (e.g., protein subunits) of at least two viral particles, preferably influenza particles. Proteins of influenza virus include, for example, expressed proteins in the virus structure, such as HA, NA, protein polymerases (PB1, PB2, PA), matrix proteins (M1, M2), and nucleoprotein (“NP”). Preferably, the conserved peptide sequences are conserved on at least two or more of the M1, M2, HA, NA, or one or more polymerase proteins.

In a preferred example, a selected sequence in the M1 and M2 proteins of the H5N1 influenza virus corresponds to the M1 and M2 proteins found in other H5N1 particles, and to the same sequence in the M1 and M2 proteins of the H3N2 influenza virus. In addition, while HA and NA proteins have highly variable regions, conserved sequences from HA and NA are found across many influenza strains and many subtypes (e.g., HA and NA sequences are conserved across H5N1 and H1N1). In a preferred embodiment of the invention, the sequences are derived from a conserved sequence present within variants or strains (viral isolates expressing substantially the same HA and NA proteins, but wherein the HA and NA protein amino acid sequences show some minor drift), of a single influenza virus subtype and more preferably across at least two influenza virus subtypes, e.g., subtypes of influenza A virus.

Peptide or polypeptide that includes at least one conserved epitope sequence, which may also comprise one or more repeats of the same or a different epitope sequence, each of which is conserved across a plurality of homologous proteins that is conserved in a population of bacterial, parasitic or viral strains or serotypes, and a pharmaceutically acceptable carrier. In exemplary composite antigens, at least one epitope sequence (continuous or discontinuous) may be repeated at least once or multiple times. Compositions may include a pharmaceutically acceptable carrier.

Peptide sequences preferably include sequences derived from genome (i.e., RNA) segment 7 of the influenza virus, while in a more preferred embodiment, the sequences include at least portions of the M1 and M2 proteins. In other preferred embodiments, the sequences include sequences expressed from genome segments encoding the HA or NA proteins. Such sequences are less affected by subtype drift and more broadly protective against infections.

Antigens may include one or more T-cell stimulating epitopes, such as diphtheria toxoid, tetanus toxoid, a polysaccharide, a lipoprotein, or a derivative or any combination thereof (including fragments or variants thereof). Typically, the at least one repeated sequence of the composite antigen is contained within the same molecule as the T-cell stimulating epitopes. In the case of protein-based T-cell stimulating epitopes, the at least one repeated sequence of the composite antigen may be contained within the same polypeptide as the T-cell stimulating epitopes, may be conjugated thereto, or may be associated in other ways. Preferably, one or more T-cell stimulating epitopes are positioned at either the N-Terminus or the C-Terminus (or both) of the antigen.

In additional embodiments, the composite antigens, with or without associated T-cell stimulating epitopes may include one or more polysaccharides or portions thereof, or one or more or multiple portions of a protein or substantially all of the immunogenic portions of a protein, wherein substantially all means sufficient to treat or prevent an infection. A preferred composition includes an immunogenic portion of A composition comprising an immunogenic portion of a peptidoglycan and an immunogenic portion of a heat shock protein. Preferably, the immunogenic portion of the peptidoglycan is obtained from a gram-positive microorganism and the gram-positive microorganism is of a spp. of Mycobacteria, a spp. of Staphylococcus, a spp. of Bacillus, or a spp. of Streptococcus. Preferably, the immunogenic portion of the peptidoglycan comprises multiple immunogenic portions of a peptidoglycan protein such as substantially all of the peptidoglycan protein. Preferably, the immunogenic portion of the heat shock protein is of a spp. of Mycobacteria such as, for example, a spp. of Mycobacteria such as M. tuberculosis, M. smegmatis, M. leprae, M. kansasii, M. mantenii, M. fortuitum, or M. xenopi. Preferably, and further the immunogenic portion is an alpha helix portion of the heat shock protein. Preferably, the immunogenic portion of peptidoglycan and the immunogenic portion of the heat shock protein are a contiguous amino acid sequence such as, for example, wherein the contiguous sequence comprises the sequence of SEQ ID NOs 148, 149, 151, or 152. Preferably, the composition includes an adjuvant which is preferably a nano-emulsion. Preferably the composition treats or prevents a gram-positive infection (e.g., Mycobacterial infection) in a mammal and may be a vaccine administered as described herein and induces opsonophagocytic killing activity against a microorganism.

In preferred embodiments, at least one sequence of a composite antigen is conjugated to one or more polysaccharides. In other embodiments, one or more polysaccharides are conjugated to other portions of the composite antigen. Certain embodiments of the present invention are selected from polysaccharide vaccines, protein-polysaccharide conjugate vaccines, protein vaccines, or combinations thereof.

Composite antigens of the invention may be synthesizing by in vitro chemical synthesis, solid-phase protein synthesis, and in vitro (cell-free) protein translation, or recombinantly engineered and expressed in bacterial cells, fungi, insect cells, mammalian cells, virus particles, yeast, and the like.

A composite antigen may include one of the following elements: at least one repeated epitope; at least one T-cell epitope; at least one polysaccharide (sugars); at least one structural component; or a combination thereof. The one structural component may include one or more of: at least one linker segment; at least one sugar-binding moiety; at least one nucleotide-binding moiety; at least one protein-binding moiety; at least one enzymatic moiety; or a combination thereof. The invention encompasses methods of preparing an immunogenic composition, preferably a pharmaceutical composition, more preferably a vaccine, wherein a target antigen of the present invention is associated with a pharmaceutically acceptable diluent, excipient, or carrier, and may be used with most any adjuvant, such as, for example, ALFQ, ALFQA, ALFA, AS01, AS01b, and/or combinations, derivatives, and modifications thereof.

Within the context of the present invention, that a relatively small number of conservative or neutral substitutions (e.g., 1 or 2) may be made within the sequence of the composite antigen or epitope sequences disclosed herein, without substantially altering the immunological response to the peptide. In some cases, the substitution of one or more amino acids in a particular peptide may in fact serve to enhance or otherwise improve the ability of the peptide to elicit a systemic response in an animal or a mammal that has been provided with a composition that comprises the modified peptide, or a polynucleotide that encodes the peptide. Suitable substitutions may generally be identified using computer programs and the effect of such substitutions may be confirmed based on the reactivity of the modified peptide with antisera and/or T-cells. Accordingly, within certain preferred embodiments, a peptide for use in the disclosed diagnostic and therapeutic methods may comprise a primary amino acid sequence in which one or more amino acid residues are substituted by one or more replacement amino acids, such that the ability of the modified peptide to react with antigen-specific antisera and/or T-cell lines or clones is not significantly less than that for the unmodified peptide.

As described above, preferred peptide variants are those that contain one or more conservative substitutions. A “conservative substitution” is one in which an amino acid is substituted for another amino acid that has similar properties, such that one skilled in the art of peptide chemistry would expect the secondary structure and hydropathic nature of the peptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Examples of amino acid substitutions that represent a conservative change include: (1) replacement of one or more Ala, Pro, Gly, Glu, Asp, Gln, Asn, Ser, or Thr; residues with one or more residues from the same group; (2) replacement of one or more Cys, Ser, Tyr, or Thr residues with one or more residues from the same group; (3) replacement of one or more Val, Ile, Leu, Met, Ala, or Phe residues with one or more residues from the same group; (4) replacement of one or more Lys, Arg, or His residues with one or more residues from the same group; and (5) replacement of one or more Phe, Tyr, Trp, or His residues with one or more residues from the same group. A variant may also, or alternatively, contain non-conservative changes, for example, by substituting one of the amino acid residues from group (1) with an amino acid residue from group (2), group (3), group (4), or group (5). Variants may also (or alternatively) be modified by, for example, the deletion or addition of amino acids that have minimal influence on the immunogenicity, secondary structure and hydropathic nature of the peptide.

Epitopes may be arranged in any order relative to one another in the composite sequence which may be with or without spacers. The number of spacer amino acids between two or more of the epitopic sequences can be of any practical range, including, for example, from 1 or 2 amino acids to 3, 4, 5, 6, 7, 8, 9, or even 10 or more amino acids between adjacent epitopes.

Another embodiment of the invention is directed to polynucleotides including DNA, RNA (e.g., cRNA, mRNA), and PNA (peptide nucleic acid) constructs that encode the composite sequences of the invention. These polynucleotides may be single-stranded (coding or antisense) or double-stranded, and may be DNA (genomic, cDNA or synthetic) or RNA molecules. Additional coding or non-coding sequences may, but need not, be present within a polynucleotide of the present invention, and a polynucleotide may, but need not, be linked to other molecules and/or support materials. As is appreciated by those of ordinary skill in the art that, as a result of the degeneracy of the genetic code, there are many nucleotide sequences that encode a given primary amino acid sequence. Some of these polynucleotides bear minimal homology to the nucleotide sequence of any native gene. Nonetheless, polynucleotides that vary due to differences in codon usage are specifically contemplated by the present invention. Polynucleotides that encode an immunogenic peptide may generally be used for production of the peptide, in vitro or in vivo. Any polynucleotide may be further modified to increase stability in vivo. Possible modifications include, but are not limited to, the addition of flanking sequences at the 5′ and/or 3′-ends; the use of phosphorothioate or 2′-o-methyl rather than phosphodiesterase linkages in the backbone; and/or the inclusion of nontraditional bases such as inosine, queosine and wybutosine, as well as acetyl- methyl-, thio- and other modified forms of adenine, cytidine, guanine, thymine and uridine.

A nucleic acid vaccine of the invention contains the genetic sequence of a composite antigen as cRNA or mRNA, or DNA, plus other necessary sequences that provide for the expression of the composite antigen in cells. By injecting the mammal with the genetically engineered nucleic acid, the composite antigen is produced in or preferably on cells, which the mammal's immune system recognizes and thereby generates a humoral or cellular response to the composite antigen, and therefore the pathogen. Nucleic acid vaccines have a number of advantages over conventional vaccines, including the ability to induce a more general and complete immune response in the mammal. Accordingly, nucleic acid vaccines can be used to protect an animal or a mammal against disease caused from many different pathogenic organisms of viral, bacterial, and parasitic origin as well as certain tumors.

Nucleic acid vaccines typically comprise a viral or bacterial nucleic acid (e.g., cRNA, mRNA, DNA) that encodes an antigen contained in vectors or plasmids that have been genetically modified to transcribe and translate the composite antigenic sequences into specific protein sequences derived from a pathogen. By way of example, the nucleic acid vaccine is administered, and the cellular machinery transcribed and/or translates the nucleic acid into the antigens which produce an immune response. The antigens, being non-natural and unrecognized by the mammalian immune system, are processed by cells and the processed proteins, preferably the epitopes, displayed on cell surfaces. Upon recognition of these antigens as foreign, the immune system generates an appropriate immune response that protects from the infection. In addition, nucleic acid vaccines of the invention are preferably codon optimized for expression in the animal (or mammal) of interest. In a preferred embodiment, codon optimization involves selecting a desired codon usage bias (the frequency of occurrence of synonymous codons in coding DNA) for the particular cell type so that the desired peptide sequence is expressed.

Compositions of the invention may contain composite antigens and epitopes, composite sequences, and/or RNA and/or DNA vaccines. Composition may include adjuvants such as, for example, oil in water emulsion, ALFQ, ALFQA, ALFA, AS01, AS01b, and/or combinations, derivatives, and modifications thereof. The formulation of pharmaceutically-acceptable excipients and carrier solutions is well known to those of ordinary skill in the art, as is the development of suitable dosing and treatment regimens for using the particular compositions described herein in a variety of treatment regimens.

The amount of immunogenic composition(s) and the time needed for the administration of such immunogenic composition(s) will be within the purview of the ordinary-skilled artisan having benefit of the present teachings. The administration of a therapeutically-effective, pharmaceutically-effective, and/or prophylactically-effective amount of the disclosed immunogenic compositions may be achieved by a single administration. Alternatively, in some circumstances, it may be desirable to provide multiple, or successive administrations of the immunogenic compositions, either over a relatively short, or even a relatively prolonged period of time, as may be determined by the skilled person overseeing the administration of such compositions.

The immunogenic compositions and vaccines of the present invention preferably contain an adjuvant such as oil in water emulsion or ALFQ and may be given by IM, SQ, Intradermal or intranasal administration or in a manner compatible with the dosage formulation, and in such an amount as will be prophylactically or therapeutically effective and preferably immunogenic. The quantity to be administered depends on the subject to be treated, including, e.g., the capacity of the immune system to mount an immune response, and the degree of protection desired. Suitable dosage ranges may be on the order of several hundred micrograms (μg) of active ingredient per animal or mammal with a preferred range from about 0.1 μg to 2000 μg (even though higher amounts, such as, e.g., in the range of about 1 to about 10 mg are also contemplated), such as in the range from about 0.5 μg to 1000 μg, preferably in the range from about 1 μg to about 500 μg and especially in the range from about 10 μg to about 100 μg. Suitable regimens for initial administration and booster shots are also variable but are typified by an initial administration followed by optional but preferred subsequent inoculations or other periodic administrations.

An effective dose comprises amounts the range of about 0.1 μg to about 1 mg total protein or target antigen per animal or mammal. In one exemplary embodiment, the vaccine dosage range is about 0.1 μg to about 10 mg per animal or mammal. However, one may prefer to adjust dosage based on the amount of peptide delivered. In either case, these ranges are merely guidelines from which one of ordinary skill in the art may deviate according to conventional dosing techniques. Precise dosages may be determined by assessing the immunogenicity of the conjugate produced in the appropriate host so that an immunologically effective dose is delivered. An immunologically effective dose is one that stimulates the immune system of the animal or mammal to establish an immune response to the immunogenic composition or vaccine. Preferably, a level of immunological memory sufficient to provide long-term protection against disease caused by microbial infection is obtained. The immunogenic compositions or vaccines of the invention may be preferably formulated with an adjuvant. By “long-term” it is preferably meant over a period of time of at least about 6 months, over at least about 1 year, over at least about 2 to 5 or even at least about 2 to about 10 years or longer. Preferably protection is provided with one administration (or one initial series of administrations) and multiple administrations over time are not required.

The following examples illustrate embodiments of the invention but are not to be viewed as limiting the scope of the invention.

### EXAMPLES

### Example 1 Peptides and Sequences

The following is a list of exemplary peptide sequences. These sequences include composite sequences as well as sequences of interest that can be combined to form composite sequences:

Influenza Virus
SEQ ID NO 1
DWSGYSGSFVQHPELTGLD (N1 sequence; H1 N5)
SEQ ID NO 2
ETPIRNE (M2e epitope)
SEQ ID NO 3
FVIREPFISCSHLEC
SEQ ID NO 4
GNFIAP (HA epitope; Pep 1)
SEQ ID NO 5
GNLIAP (HA epitope; Pep 2)
SEQ ID NO 6
GNLFIAP (composite sequence of SEQ ID NOs 4 and 5; Pep 3)
SEQ ID NO 7
GNLIFAP (composite sequence of SEQ ID NOs 4 and 5)
SEQ ID NO 8
HYEECSCY (NA epitope; Pep 10)
SEQ ID NO 9
LLTEVETPIR (highly conserved region M1/M2e)
SEQ ID NO 10
LLTEVETPIRN (highly conserved region M1/M2e)
SEQ ID NO 11
LLTEVETPIRNE (highly conserved region M1/M2e)
SEQ ID NO 12
DWSGYSGSFVQHPELTGL (N1 sequence; H1 N5)
SEQ ID NO 13
EVETPIRNE (highly conserved region M1/M2e)
SEQ ID NO 14
FLLPEDETPIRNEWGLLTDDETPIRYIKANSKFIGITE
SEQ ID NO 15
GNLFIAPGNLFIAPHYEECSCYHYEECSCYQYIKANSKFIGITEHY
EECSCYTPIRNETPIRNE
SEQ ID NO 16
GNLFIAPGNLFIAPQYIKANSKFIGITEGNLFIAP (composite of SEQ
ID NO 6, SEQ ID NO 6, SEQ ID NO 61, and SEQ ID NO 6)
SEQ ID NO 17
HYEECSCYDWSGYSGSFVQHPELTGLHYEECSCYQYIKAN
SKFIGITE
SEQ ID NO 18
ITGFAPFSKDNSIRLSAGGDIWVTREPYVSCDP
SEQ ID NO 19
IWGIHHP (HA epitope)
SEQ ID NO 20
IWGVHHP (HA epitope)
SEQ ID NO 21
IWGVIHHP (composite of SEQ ID NOs. 19 and 20)
SEQ ID NO 22
IWGIVHHP (composite of SEQ ID NOs. 19 and 20)
SEQ ID NO 23
KSCINRCFYVELIRGR (N3 conserved epitope)
SEQ ID NO 24
LLTEVETPIRNESLLTEVETPIRNEWG (M2e epitope)
SEQ ID NO 25
LLTEVETPIRNEW (M2e epitope)
SEQ ID NO 26
LLTEVETPIRNEWG (M2e epitope)
SEQ ID NO 27
LTEVETPIRNE (M2e epitope)
SEQ ID NO 28
LTEVETPIRNEW (M2e epitope)
SEQ ID NO 29
LTEVETPIRNEWG (M2e epitope)
SEQ ID NO 30
MSLLTEVET (M2e epitope)
SEQ ID NO 31
MSLLTEVETP (M2e epitope)
SEQ ID NO 32
MSLLTEVETPI (M2e epitope)
SEQ ID NO 33
MSLLTEVETPIR (M2e epitope)
SEQ ID NO 34
MSLLTEVETPIRN (M2e epitope)
SEQ ID NO 35
MSLLTEVETPIRNE (M2e epitopes)
SEQ ID NO 36
MSLLTEVETPIRNETPIRNE (M2e epitope)
SEQ ID NO 37
MSLLTEVETPIRNEW (M2e epitope)
SEQ ID NO 38
MSLLTEVETPIRNEWG (M2e epitope)
SEQ ID NO 39
MSLLTEVETPIRNEWGCRCNDSSD (M2e epitope)
SEQ ID NO 40
SLLTEVET (M2e epitope)
SEQ ID NO 41
SLLTEVETPIR (M2e epitope)
SEQ ID NO 42
SLLTEVETPIRNE (M2e epitope)
SEQ ID NO 43
SLLTEVETPIRNEW (M2e epitope)
SEQ ID NO 44
SLLTEVETPIRNEWG (M2e epitope)
SEQ ID NO 45
SLLTEVETPIRNEWGTPIRNE (M2e epitope)
SEQ ID NO 46
SLLTEVETPIRNEWGTPIRNETPIRNE (M2e epitope)
SEQ ID NO 47
SLLTEVETPIRNEWGTPIRNETPIRNETPIRNE (M2e epitopes)
SEQ ID NO 48
SLLTEVETPIRNEWGLLTEVETPIR (M1/M2e conserved region)
SEQ ID NO 49
TEVETPIRNE (M2e epitope)
SEQ ID NO 50
TPIRNE (M2e epitope)
SEQ ID NO 51
VETPIRNE (M2e epitope)
SEQ ID NO 52
VTREPYVSCDPKSCINRCFYVELIRGRVTREPYVSCDPWYIK
ANSKFIGITE
SEQ ID NO 53
WGIHHP (HA conserved region; Pep 5)
SEQ ID NO 54
WGVHHP (HA conserved region; Pep 4)
SEQ ID NO 55
WGVIHHP (composite of SEQ ID NOs 53 and 54; Pep 6)
SEQ ID NO 56
WGIVHHP (composite of SEQ ID NOs 53 and 54; Pep 7)
SEQ ID NO 57
YIWGIHHP (HA conserved region)
SEQ ID NO 58
YIWGVHHP (HA conserved region)
SEQ ID NO 59
YIWGVIHHP (composite of SEQ ID NOs 57 and 58)
SEQ ID NO 60
YIWGIVHHP (composite of SEQ ID NOs 57 and 58)
SEQ ID NO 61
QYIKANSKFIGITE (T cell epitope)
SEQ ID NO 62
PIRNEWGCRCNDSSD (M2e epitope)
SEQ ID NO 63
GNLFIAPWGVIHHPHYEECSCY (underlined sequences are epitopes
HA{composite} (SEQ ID NO 6) and NA (SEQ ID NO 8), respectively,
with middle as SEQ ID NO 55, of Influenza A; Pep 11)
SEQ ID NO 64
CAGAGNFIAP
SEQ ID NO 65
CAGAGNLIAP
SEQ ID NO 66
CAGAGNLFIAP
SEQ ID NO 67
CAGAWGVHHP
SEQ ID NO 68
CAGAWGIHHP
SEQ ID NO 69
CAGAWGVIHHP
SEQ ID NO 70
CAGAWGIVHHP
SEQ ID NO 71
GNLIAPWGVIHHP
SEQ ID NO 72
CAGAGNLIAPWGVIHHP
SEQ ID NO 73
GNLFIAPWGVIHHP
SEQ ID NO 74
CAGAGNLFIAPWGVIHHP
SEQ ID NO 75
HYEECSCY
SEQ ID NO 76
CAGAHYEECSCY
SEQ ID NO 77
CAGAGNLFIAPWGVIHHPHYEECSCY
SEQ ID NO 78
GNLFIAPWGVIHHPGNLFIAPWGVIHHP
SEQ ID NO 79
CAGAGNLFIAPWGVIHHPGNLFIAPWGVIHHP
SEQ ID NO 80
HYEECSCYGNLFIAPWGVIHHP
SEQ ID NO 81
GNLFIAPHYEECSCYWGVIHHP
SEQ ID NO 82
SLLTEVETPIRNEWGLLTEVETPIRQYIKANSKFIGITE
SEQ ID NO 83
GNLFIAPGNLFIAPQYIKANSKFIGITEGNLFIAP
SEQ ID NO 84
HYEECSCYDWSGYSGSFVQHPELTGLHYEECSCYQYIKANSKFIGITE
SEQ ID NO 85
VTREPYVSCDPKSCINRCFYVELIRGRVTREPYVSCDPQYIKANSKFIGITE
SEQ ID NO 86
DWSGYSGSFVQHPELTGL
SEQ ID NO 87
ITGFAPFSKDNSIRLSAGGDIWVTREPYVSCDP
SEQ ID NO 88
KSCINRCFYVELIRGR
SEQ ID NO 89
GNLFIAPRYAFA
SEQ ID NO 90
CAGAGNLFIAPRYAFA
SEQ ID NO 91
GNLVVPRYAFA
SEQ ID NO 92
CAGAGNLVVPRYAFA
SEQ ID NO 93
GNLIAPRYAFA
SEQ ID NO 94
CAGAGNLIAPRYAFA
SEQ ID NO 95
GNLVVP
SEQ ID NO 96
CAGAGNLVVP
SEQ ID NO 97
FVIREPFISCSHLEC
SEQ ID NO 98
CAGAFVIREPFISCSHLEC
SEQ ID NO 99
GNLFIAPWGVIHHPHYEECSCY (Pep 11)
SEQ ID NO 100
GNLFIAPWGVIHHPHYEECSCYQYIKANSKFIGITE
(Pep 11 with C terminal T cell epitope = Pep 63)
SEQ ID NO 101
QYIKANSKFIGITEGNLFIAPWGVIHHPHYEECSCY
(Pep 11 with N terminal T cell epitope = Pep 64)
SEQ ID NO 102
HVEECSY (N1 and N2)
SEQ ID NO 103
WFIHHP (H5)
SEQ ID NO 104
DLWSYNAELLV (stem peptide)
SEQ ID NO 105
DIWTYNAELLV (stem peptide)
SEQ ID NO 106
HXXXW-matrix peptide common to Flu A and B that constitutes the
main functional element of the M2 channel
Coronavirus
SEQ ID NO 107
YPKCDRA = RNA Polymerase region
SEQ ID NO 108
WDYPKCDRA = RNA Polymerase region
SEQ ID NO 109
SLDQINVTFLDLEYEMKKLEESY w/ QYIKANSKFIGITE = spike
protein w/ tetanus toxoid T cell epitope
Five coronavirus composite sequences using conserved epitopes.
SEQ ID NO 110
SLDQINVTFLDLEYEMKKLEESY
(coronavirus spike protein conserved epitope (SP))
SEQ ID NO 111
SLDQINVTFLDLEYEMKKLEESYQYIKANSKFIGITE
(tetanus toxoid T cell epitope + SP)
SEQ ID NO 112
WDYPKCDRA
(polymerase conserved epitope (POL))
SEQ ID NO 113
WDYPKCDRAQYIKANSKFIGITE
(POL + tetanus T cell epitope)
SEQ ID NO 114
WDYPKCDRASLDQINVTFLDLEYEMKKLEESYQYIKANSKFIGITE
(Cor POL + SP + Tet)
SEQ ID NO 115
WDYPKCDRATEVETPIRNEHYEECSCYQYIKANSKFIGITE
Cor POL. Flu M2e. Flu NA. Tetanus T cell
(One coronavirus conserved epitope and two Flu conserved epitopes that
is a broader pandemic vaccine)
Coronavirus Peptides and Composite Coronavirus/Influenza Peptides
SEQ ID NO 116
SLDQINVTFLDLEYEMKKLEESYQYIKANSKFIGITE
(spike protein epitope with T-cell stimulating epitope)
SEQ ID NO 117
WDYPKCDRA (corona conserved seq-polymerase) neutralizing Ab
SEQ ID NO 118
YPKCDRA (corona conserved seq-polymerase)
SEQ ID NO 119
ARDLICAQ
(highly conserved cor seq-spike attachment same is all three-Cor
MERSSARS)
SEQ ID NO 120
KWPWYIWLGFIAGL (highly conserved cor seq-spike attachment)
SEQ ID NO 121
ENQKLIAN (highly conserved cor seq-spike attachment)
SEQ ID NO 122
ARDLICAQKWPWYIWLGFIAGLENQKLIAN
(combination of conserved seqs w/o T cell epitope)
SEQ ID NO 123
ENQKLIANARDLICAQ
(combination of conserved seqs w/o T cell epitope)
SEQ ID NO 124
WDYPKCDRAENQKLIANARDLICAQ
(combination of conserved seqs w/o T cell epitope)
SEQ ID NO 125
WDYPKCDRAENQKLIANKWPWYIWLGFIAGL
(combination of conserved seqs w/o T cell epitope)
SEQ ID NO 126
ARDLICAQENQKLIANWDYPKCDRAQYIKANSKFIGITE
(combinations of cor conserved seqs w/ T cell epitope)
SEQ ID NO 127
KWPWYIWLGFIAGLWDYPKCDRAQYIKANSKFIGITEARDLIC
AQENQKLIANWDYPKCDRAQYIKANSKFIGITE
(combination of cor conserved seqs w/ T cell epitope)
SEQ ID NO 128
ARDLICAQENQKLIANQYIKANSKFIGITE ARDLICAQENQKLIAN
WDYPKCDRAQYIKANSKFIGITE (combination of cor conserved seqs w/ T cell epitope)
SEQ ID NO 129
WDYPKCDRATEVETPIRNEHYEECSCYQYIKANSKFIGITE
ARDLICAQENQKLIANWDYPKCDRAQYIKANSKFIGITE
(combination of cor plus Influenza conserved seqs w/ T cell epitope)
Just bold = Cor
Italicized = m2e
Underlined = Flu
Bold and underlined-T-cell
SEQ ID NO 130
HYEECSCYWDYPKCDRAVETPIRNEQYIKANSKFIGITE
(combination of cor plus Influenza conserved seqs w/ T cell epitope)
SEQ ID NO 131
ENQKLIANTEVETPIRNEHYEECSCYQYIKANSKFIGITE
(combination of cor plus Influenza conserved seqs w/ T cell epitope)
Universal Influenza Virus Vaccine Peptides
SEQ ID NO 132
GNLFIAPWGVIHHPHYEECSCYTEVETPIRNEQYIKANSKFIGITE
(Influenza composite epitopes of two HA conserved regions plus NA and
matrix epitopes with T cell epitope)
SEQ ID NO 133
QYIKANSKFIGITEGNLFIAPWGVIHHPHYEECSCYTEVETPIRNE
(T cell epitope and Influenza composite epitopes of two HA conserved
regions, plus NA and matrix epitopes)
Any combination of SEQ ID Nos.

### Example 2 Induction of Neutralizing Antibodies with Composite Peptides

ICR mice and cotton rats were immunized with 1 μg of conjugated or unconjugated composite influenza peptide vaccine (influenza HA, NA and M2e composite peptides with a T-cell epitope) formulated with ALFQ by intramuscular, or intradermal routes (cotton rats were given both intramuscular, or intradermal injections). Both routes of administration induced serum IgG antibodies that bound to groups 1 and 2 influenza viruses (Flu A California H1N1/pdm09 and Flu A Hong Kong H3N2/4801/2014). In addition, 1 μg of composite influenza vaccine formulated in ALFQ induced neutralizing antibodies against both influenza viruses given by intradermal and intramuscular routes. These data demonstrate that composite influenza peptide vaccines formulated in ALFQ induced a strong immune response at a very low dose without conjugation to a carrier and when administered by different routes of immunization. This provides an advantage in efficiency of manufacturing and decreased cost of production. Low dose intradermal administration also decreases vaccine costs for mass global immunization of humans and for immunizing mammals such as humans or animals such as birds and pigs.

### Example 3 Universal Influenza Vaccine

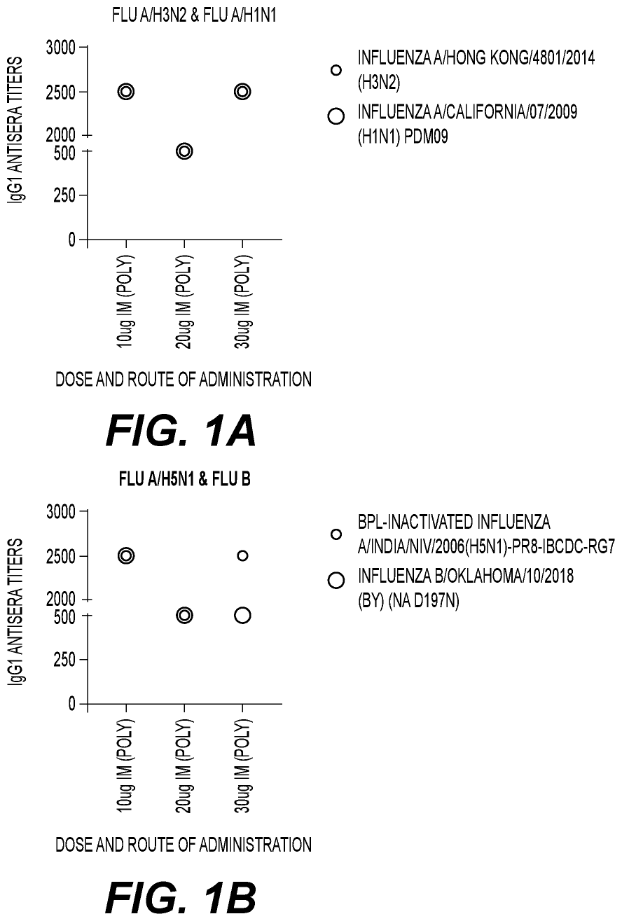

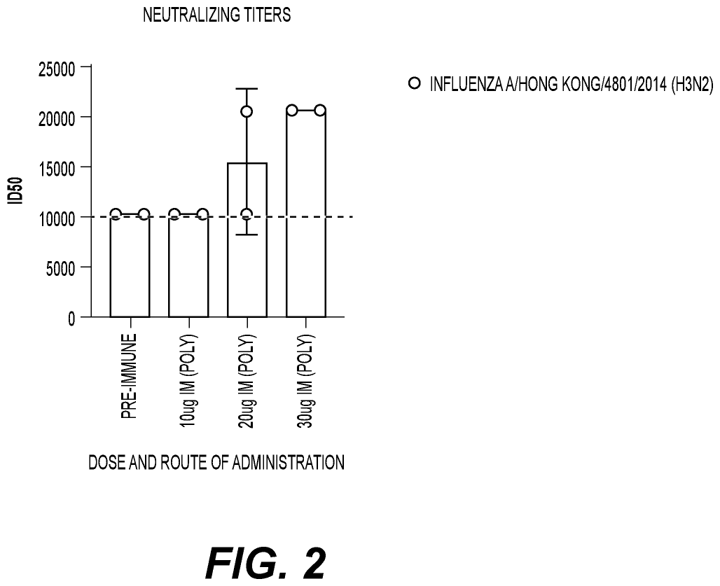

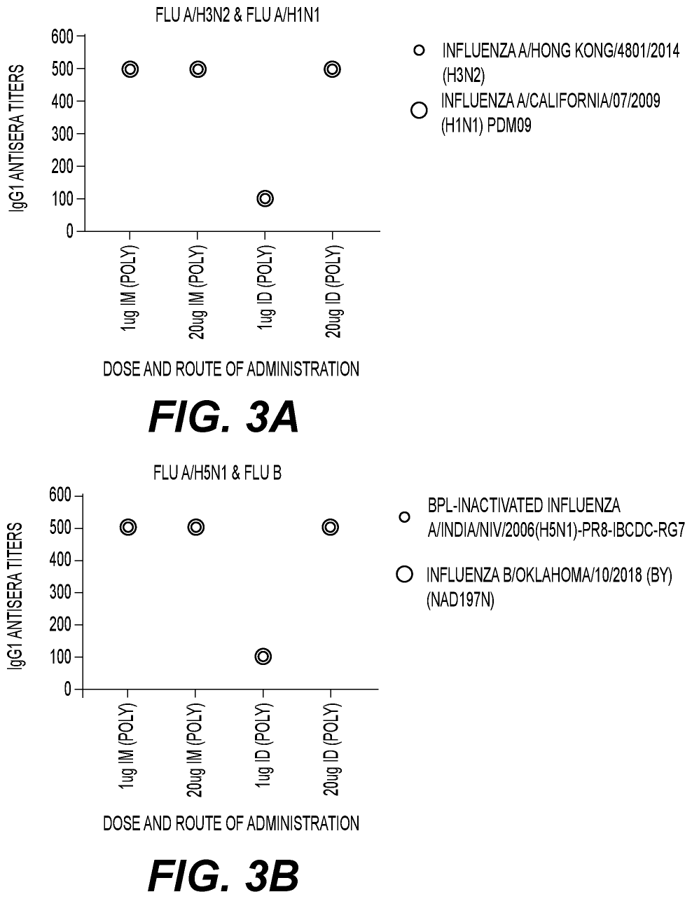

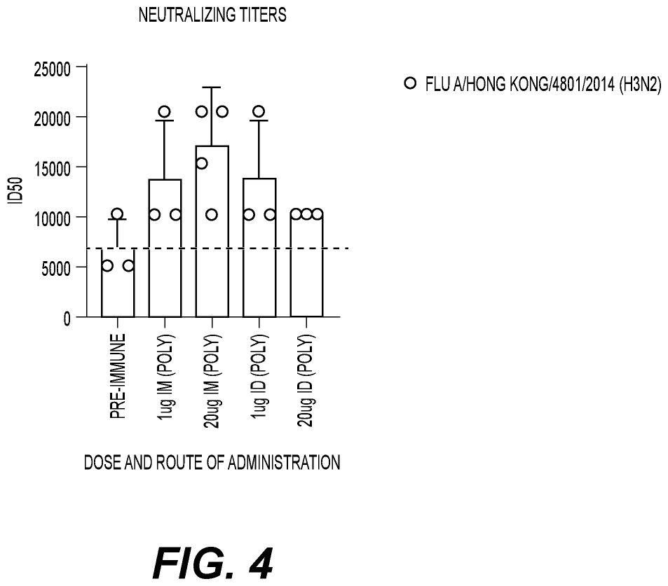

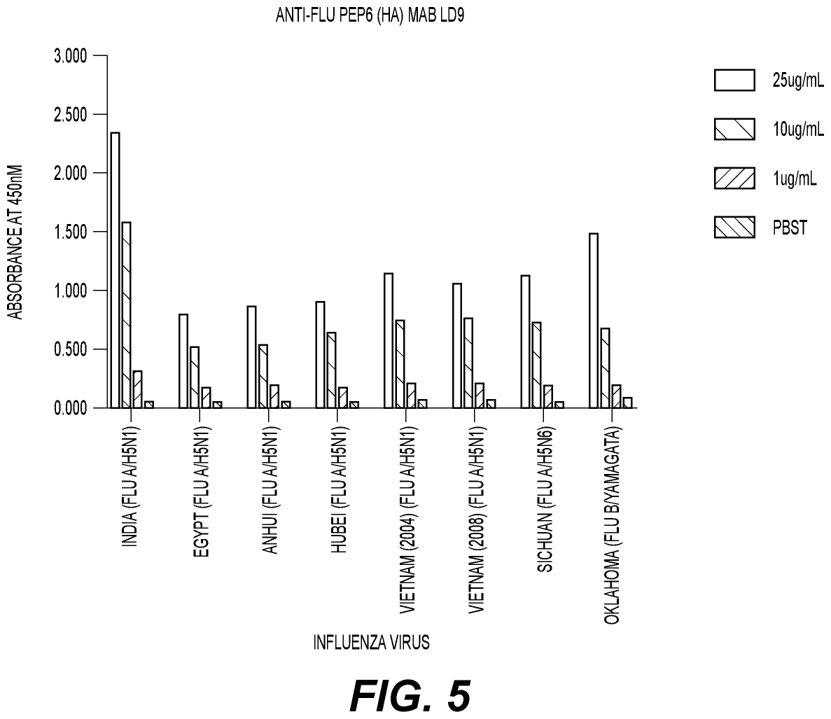

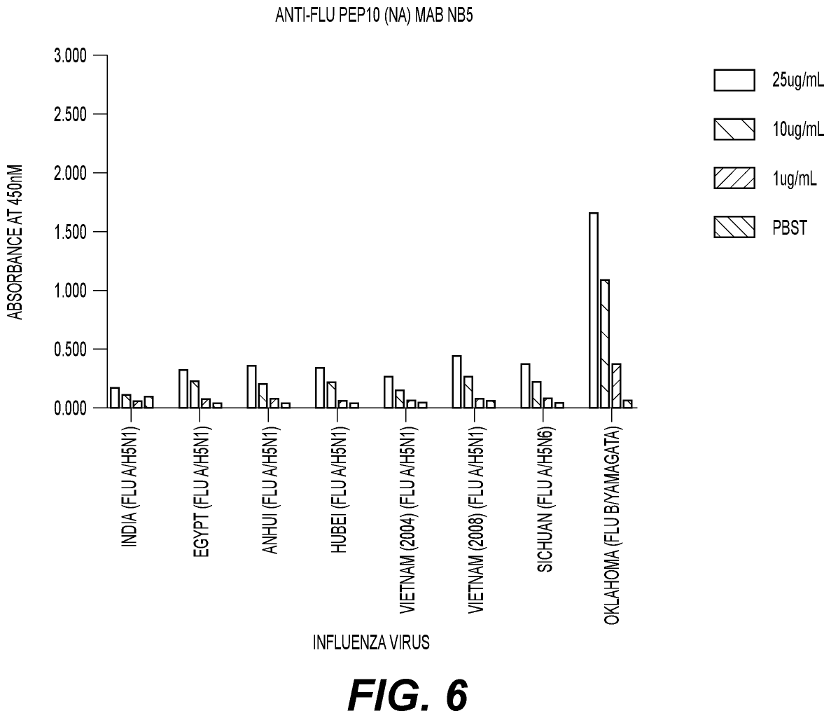

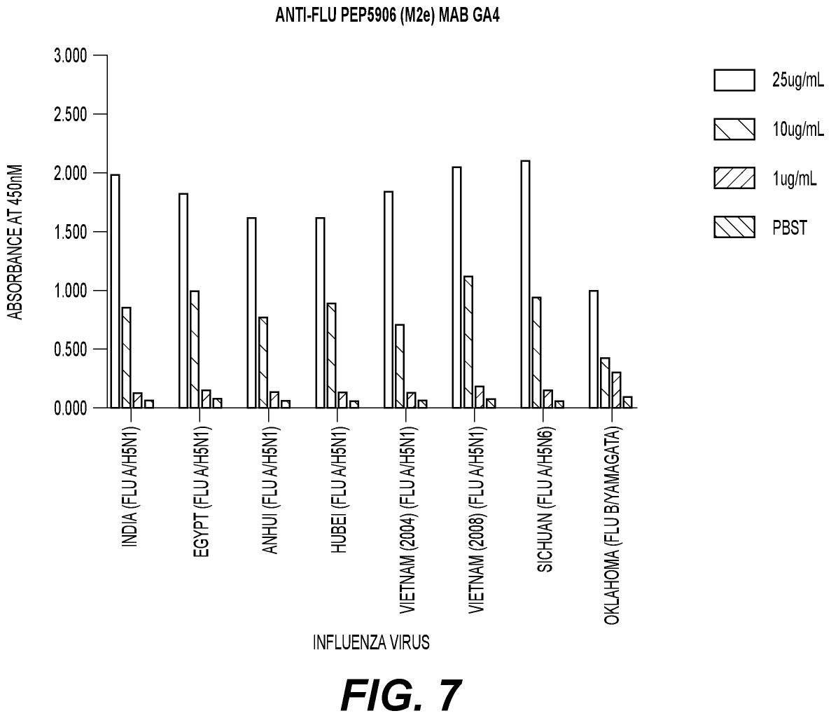

The composite influenza peptide vaccine induced serum antibodies that bind broadly across groups 1 and 2 influenza A viruses (to include pandemic and avian influenza strains) and influenza B virus (FIGS. 1 and 3). The composite influenza peptide vaccine induced serum antibodies that neutralize influenza A viruses (FIGS. 2 and 4). As shown in FIG. 5, anti-Flu Pep 6 MAB LD9 targets Hemagglutinin (HA) and binds to pandemic and avian influenza strains and influenza B virus. As shown in FIG. 6, anti-Flu Pep 10 MAB NB5 targets Neuraminidase (NA) and binds to pandemic and avian influenza strains and influenza B virus. As shown in FIG. 7, anti-Flu Pep 5906 MAB GA4 targets Matrix Ectodomain (M2e), binds to pandemic and avian influenza strains and influenza B virus. Neutralizing MABs LD9, NB5, and GA4 that bind to these influenza peptide epitopes (HA, NA, and M2e, respectively) target both seasonal influenza A strains and pandemic/avian influenza strains to include H5N1 and H5N6 (FIGS. 5, 6 and 7). In addition, the neutralizing MABs to these peptides also bind strongly to influenza B (FIGS. 1, 3, 5, 6, 7).

### Example 4 Composite Peptide TB, Gram Positive Bacteria and Influenza/Coronavirus Vaccines

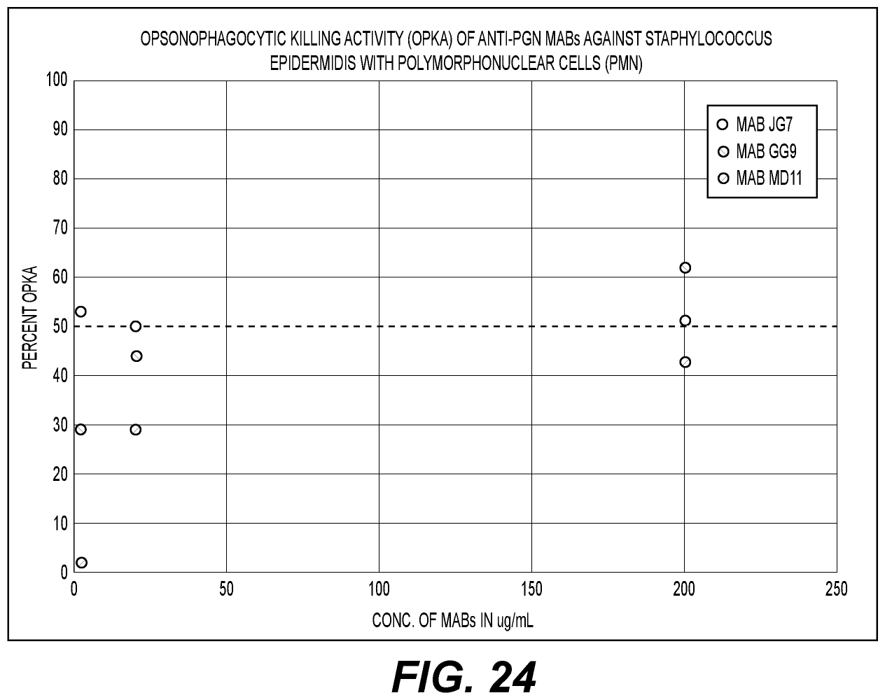

The 16.3 KD alpha crystallin heat shock protein (HSP16.3) belongs to the small heat shock protein (HSP20) family. It plays a major role for MTB survival, growth, virulence and cell wall thickening. TB Pep 01 is a highly conserved region of HSP16.3 and immunization of mice induced antibodies that bind to mycobacteria and promote opsonophagocytic killing of M. smegmatis (FIGS. 24-27). Peptidoglycan is a cell wall component that is common across many bacteria and antibodies to PGN bind to MTB (and other gram-positive bacteria). Immunization of mice with ethanol killed MTB induced anti-PGN antibodies that promoted phagocytic killing of MTB. In addition, these antibodies bind to small PGN epitopes (Table 2) and composite antigens (SEQ ID NO 132-139). Cell wall PGN (SEQ ID NO 132) composite peptides and HSP16.3 (SEQ ID NO 148) the highly conserved peptide (TB Pep 01) can be mixed and matched to produce composite peptides and mixtures with or without an added T cell epitope to provide vaccines to produce broadly protective immunity across large groups of bacteria.

TABLE 2
PGN Peptide Sequences

| SEQ | Peptide | | |
|---|---|---|---|
| ID NO | number | Peptide ID | Peptide Sequence |
| 132 | PGN Pep01 | LVD-PSEQ-A-PGN Pep | AEKAGGGGGAEKA |
| | | 01 | |
| 133 | PGN Pep02 | LVD-PSEQ-A-PGN Pep | AEKAEKAGGGGGAEKAEKA |
| | | 02 | |
| 134 | PGN Pep03 | LVD-PSEQ-A-PGN Pep | QYIKANSKFIGITEAEKAGGGGAEK |
| | | 03 | A |
| 135 | PGN Pep04 | LVD-PSEQ-A-PGN Pep | AEKAGGGGGAEKAQYIKANSKFIGI |
| | | 04 | TE |
| 136 | PGN Pep05 | LVD-PSEQ-A-PGN Pep | AEKA |
| | | 05 | |
| 137 | PGN Pep06 | LVD-PSEQ-A-PGN Pep | AEKAGGGGG |
| | | 06 | |

SEQ ID NO 138: QYIKANSKFIGITE = tetanus universal T cell epitope
SEQ ID NO. 139: GGGGG = pentaglycine bridge

In addition, combining HSP16.3 with PGN epitopes provides a TB vaccine that targets active MTB infection and latency. This vaccine could be used alone, or in combination with BCG and could be used as a booster vaccine with BCG, or other TB vaccines. In a similar fashion, LTA mimotopes combined with PGN epitopes (Table 3) provide an example of a broad composite peptide gram positive bacterial vaccine, while mixing coronavirus and influenza peptides provides a prototype composite peptide vaccine for prevention or treatment of infections by these viruses.

TABLE 3
MTB, PGN, and LTA Composite Peptide Sequences

| SEQ ID | Peptide | | |
|---|---|---|---|
| NO | number | Peptide ID | Peptide Sequence |
| 148 | TB Pep01 | LVD-PSEQ- | SEFAYGSFVRTVSLPVGADE |
| | | A-TB Pep 01 | |
| 149 | PGN.TB | LVD-PSEQ- | AEKAGGGGGAEKASEFAYGSFVRTVSLPVGADE |
| | Pep01 | A-PGN.TB | |
| | | Pep01 | |
| 150 | PGN.TB | LVD-PSEQ- | AEKAGGGGGAEKASEFAYGSFVRTVSLPVGADEQYIKAN |
| | Pep02 | A-PGN.TB | SKFIGITE |
| | | Pep02 | |
| 151 | PGN.TB | LVD-PSEQ- | SEFAYGSFVRTVSLPVGADEAEKAGGGGGAEKA |
| | Pep03 | A-PGN.TB | |
| | | Pep03 | |
| 152 | PGN.TB | LVD-PSEQ- | AEKAGGGGGSEFAYGSFVRTVSLPVGADEGGGGGAEKA |
| | Pep04 | A-PGN.TB | |
| | | Pep04 | |
| 153 | PGN.TB | LVD-PSEQ- | AEKAGGGGGSEFAYGSFVRTVSLPVGADEGGGGGAEKA |
| | Pep05 | A-PGN.TB | QYIKANSKFIGITE |
| | | Pep05 | |
| 154 | PGN.LTA | LVD-PSEQ- | WRMYFSHRHAHLRSPGGGGGAEKAGGGGGQYIKANSKFI |
| | Pep01 | A-PGN.LTA | GITE |
| | | Pep01 | |
| 155 | PGN.LTA | LVD-PSEQ- | WHWRHRIPLQLAGRAEKAGGGGGWRMYFSHRHAHLRSP |
| | Pep02 | A-PGN.LTA | QYIKANSKFIGITE |
| | | Pep02 | |

Description of sequences listed in Table 3.
148. TB Pep 01-MTB 16.3HSP Conserved Region (CR).
149-153. PGN epitopes and MTB 16.3HSP (CR) with and without a T cell epitope.
154 & 155. PGN and LTA peptides with a T cell epitope.

Studies in ICR mice have also demonstrated that immunization with unconjugated TB Pep 1 plus PGN formylated with ADDAVAX™ adjuvant induced robust serum antibody responses with doses as low as 10-20 μg of each peptide/antigen. Antibodies broadly targeted bacteria to include Mycobacteria, Staphylococci, Streptococci, and Bacillus species. In addition, antibodies were shown to promote opsonophagocytic killing of bacteria by U937 macrophages. This HSP16.3 and PGN vaccine that covers multiple pathogens provides a cost effective and easily scalable approached for a vaccine to target TB and gram-positive pathogens.

### Example 5 MABs to PGN are Opsonic

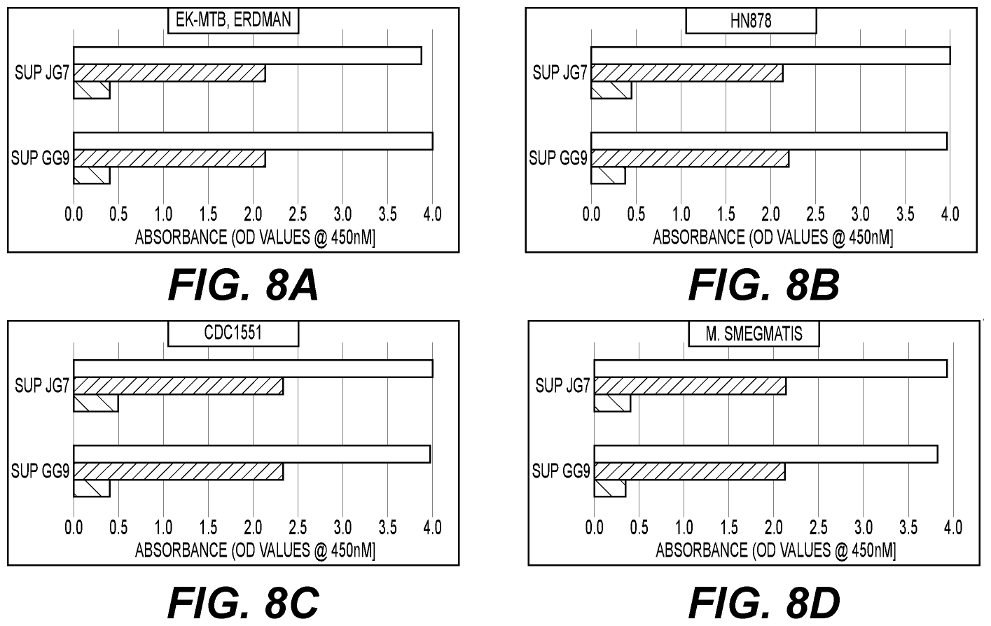

MABs JG7 and GG9 showed binding activity to killed MTB, live Mycobacterium smegmatis (SMEG) and several strains of live MTB-susceptible, MDR and XDR. In addition, JG7 and GG9 promoted opsonophagocytic killing of SMEG and MTB using macrophage and granulocytic cell lines and enhanced clearance of MTB from blood (see FIGS. 8-14).

Monoclonal antibodies (MABs) JG7, GG9, and MD11 were developed against a Mycobacterium tuberculosis (MTB) and gram-positive bacteria cell wall component peptidoglycan (PGN). Mouse splenocytes were fused with SP2/0 myeloma cells for production of hybridomas and MABs. MAB JG7 (IgG1) was derived from BALB/c MS 1323 immunized intravenously with Ethanol-killed Mycobacterium tuberculosis (EK-MTB), without adjuvant. Killing of MTB using Ethanol may have altered the MTB capsule exposing deeper cell wall epitopes. MAB GG9 (IgG1) was derived from BALB/c MS 1420 immunized subcutaneously with EK-MTB, without adjuvant. MAB MD11 (IgG2b) was derived from ICR MS 190 immunized subcutaneously with ultrapure Peptidoglycan (PGN), conjugated to CRM197 and adjuvanted with TiterMax® Gold. EK-MTB and PGN were immunogenic in mice. Serum antibodies that bound to gram-positive bacteria and MTB and promoted opsonophagocytic killing (OPKA) of the bacteria by phagocytic effector cells.

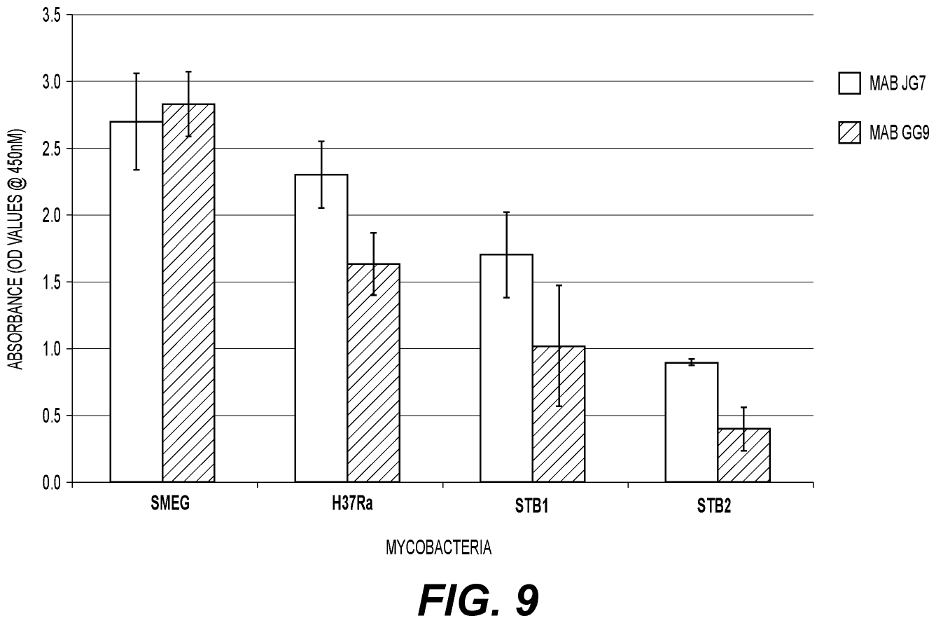

Binding activities of supernatants from hybridomas JG7 and GG9 (from mice 1323 and 1420, respectively), to Mycobacterium tuberculosis (MTB) and Mycobacterium smegmatis (SMEG), evaluated at dilutions 1:10, 1:100, and 1:1000 on fixed mycobacteria at 1×10⁵ CFU/well. FIG. 8A, FIG. 8B FIG. 8C, respectively, shows binding of supernatant to killed MTB Erdman, HN878 and CDC1551. FIG. 8D depicts binding of supernatants to fixed SMEG. OD values for growth media without antibody (negative control) range between 0.046-0.060. Binding activity of purified anti-Mycobacterium tuberculosis monoclonal antibodies (anti-MTB MABs) GG9 and JG7 to live Mycobacterium smegmatis (SMEG) and live susceptible MTB H37Ra (lab strain) and STB1 and STB2 (susceptible clinical isolates) was demonstrated in a live bacteria ELISA (FIG. 9). Data (expressed as mean±standard errors; n=3) are representative of three individual experiments.

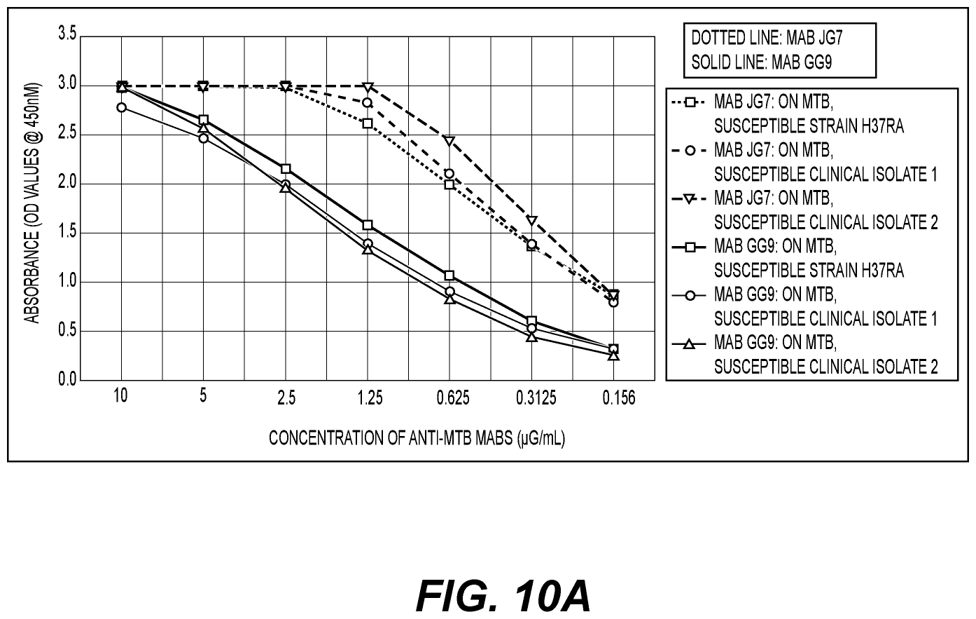

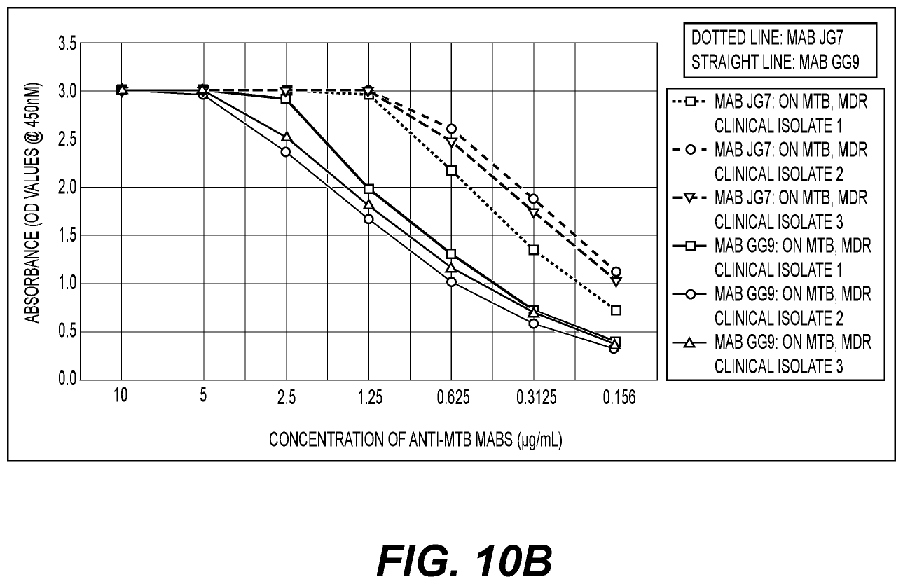

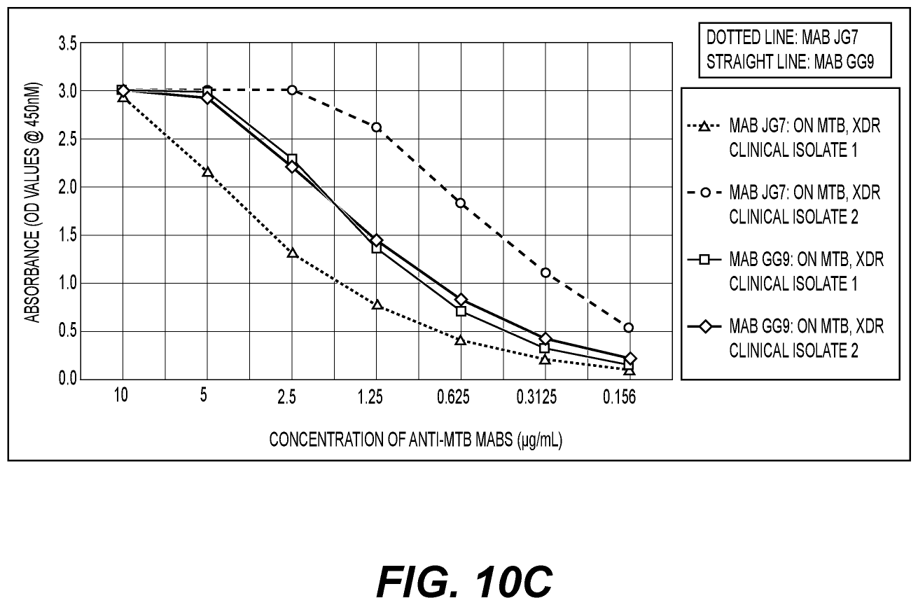

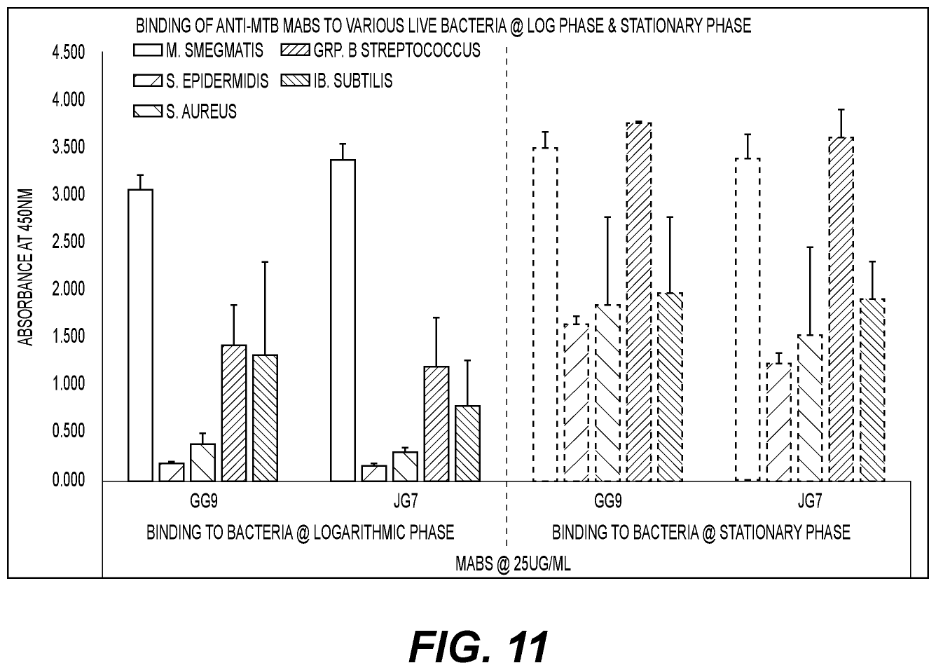

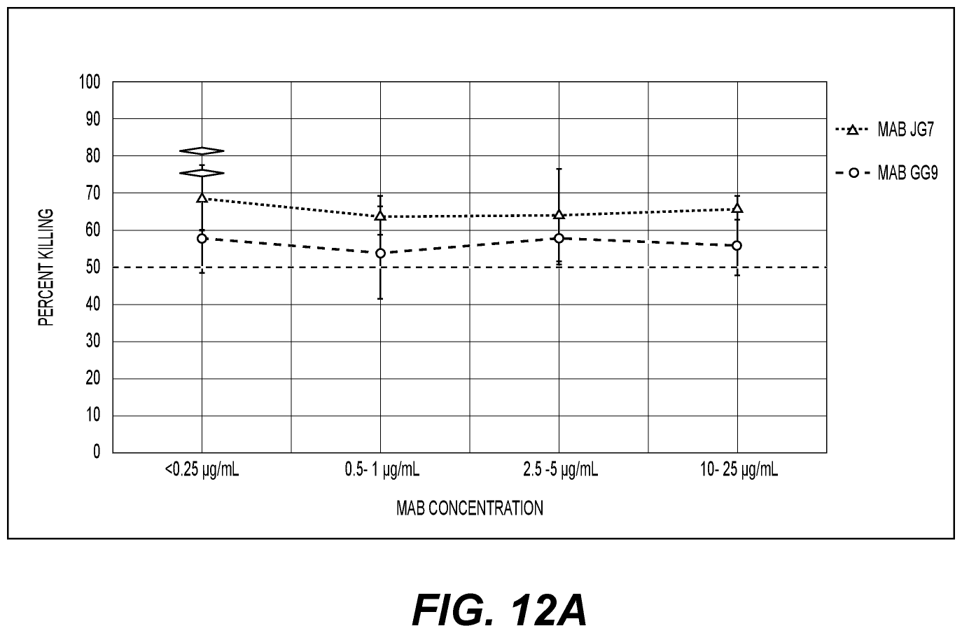

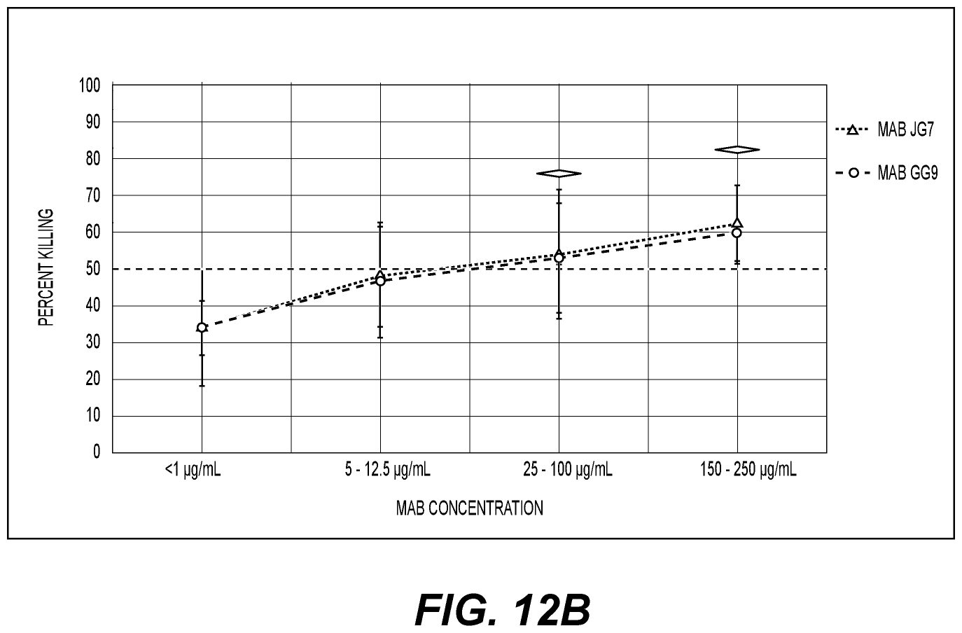

FIGS. 10A-C demonstrate binding activity of purified anti-Mycobacterium tuberculosis monoclonal antibodies (anti-MTB MABs) JG7 and GG9 to fixed MTB at 1×10⁵ CFU/well. FIG. 10A demonstrates MAB binding to susceptible H37Ra strain and clinical isolates 1, and 2; FIG. 10B to multidrug-resistant (MDR) clinical isolates 1, 2 & 3; and FIG. 10C to extensively drug-resistant (XDR) clinical isolates 1 and 2. Data (expressed as mean) are representative of three individual experiments. FIG. 11 demonstrates binding activity of anti-MTB MABs JG7 & GG9 to various live gram-positive bacteria grown to either log phase or stationary phase as screened in the live bacteria ELISA. As shown in FIGS. 12A-B, enhanced OPKA of MABS JG7 and GG9 against Mycobacterium smegmatis (SMEG) using HL60 granulocytes and C1q (FIG. 12A) occurred at low antibody concentrations (<0.25 μg/ml) and stayed constant when antibody levels were increased over one hundred-fold. While MAB JG7 consistently had higher percent killing, the difference did not reach statistical significance. Peak OPKA for both JG7 and GG9 occurred at 0.06 μg/mL and were 81% and 76%, respectively. In FIG. 12B, enhanced MAB OPKA against SMEG using U-937 macrophages (without C1q) was significantly more pronounced at higher antibody concentrations (JG7: p=0.0001, GG9: p<00001) and both MABs tracked closely together across all antibody concentrations. Peak OPKA for JG7 and GG9 were 82% at 175 μg/mL and 76% at 100 μg/mL, respectively.

To summarize, these supernatants bound to multiple MTB strains (FIGS. 8A-C), M. smegmatis (FIG. 8D), and susceptible, MDR, and XDR clinical isolates (FIGS. 9-12).

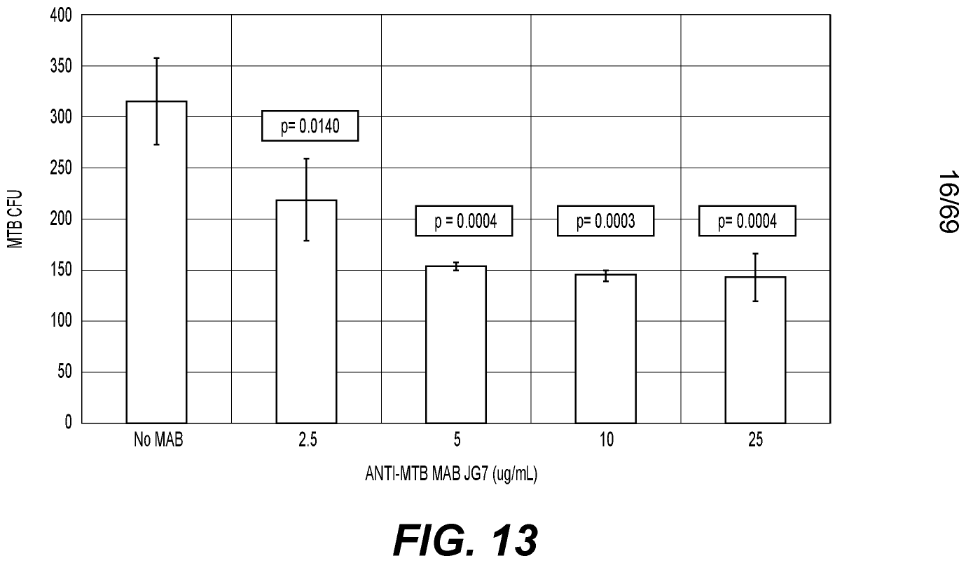

As shown in FIG. 13, OPKA of MAB JG7 against live Mycobacterium tuberculosis (MTB) clinical isolate STB1, using U-937 macrophages (without C1q) was significantly enhanced at MAB levels 2.5-25 μg/mL. Compared to the control sample wells (without MAB), antibody sample wells had CFU counts that were significantly reduced (p<0.5) from 315 (No MAB) to 219 (2.5 μg/mL), 154 (5 μg/mL), 145 (10 μg/mL) and 143 (25 μg/mL). Data (expressed as mean±standard errors; n=3) are representative of three individual experiments. The MABs also demonstrated broad bacterial binding and enhanced OPKA against MTB and M. smegmatis (FIG. 13).

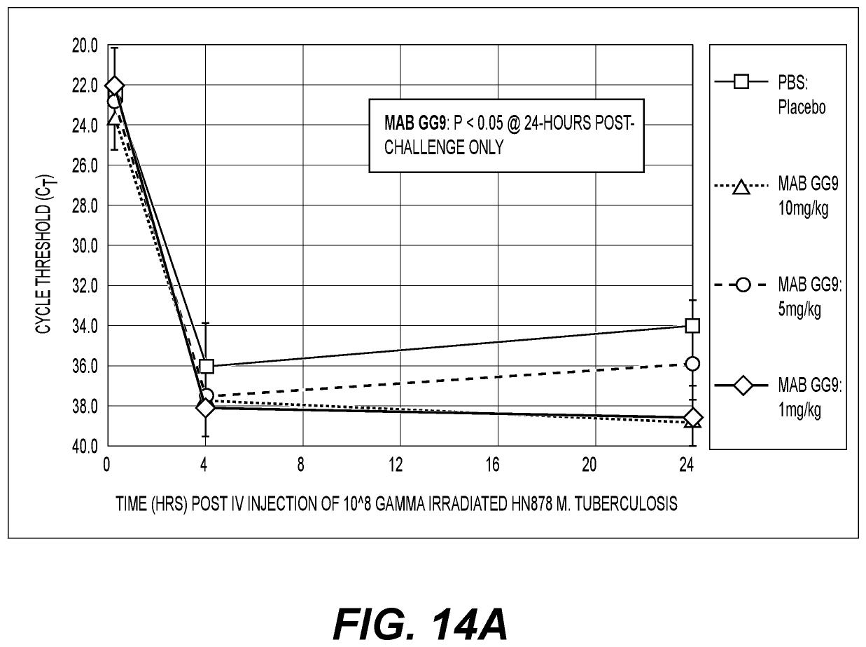

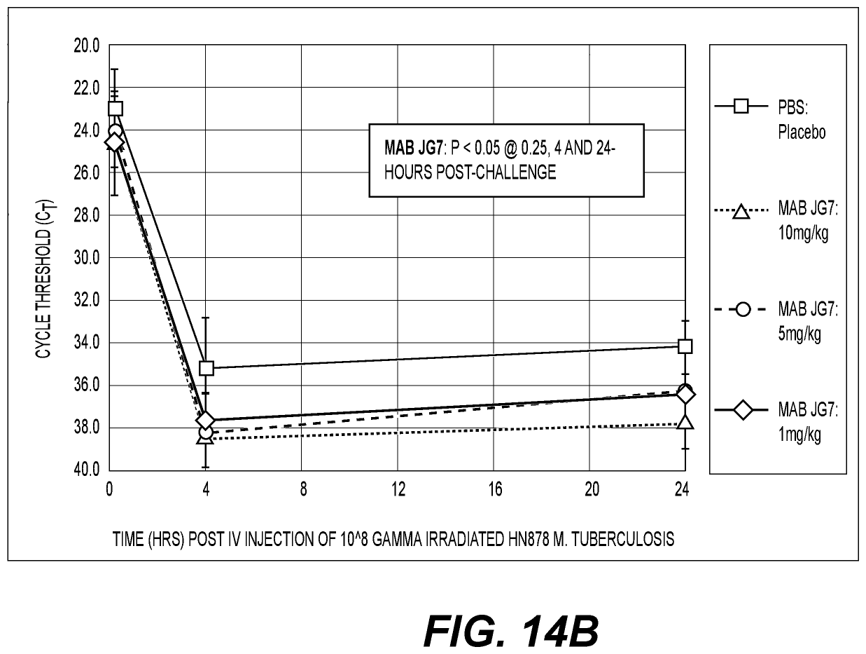

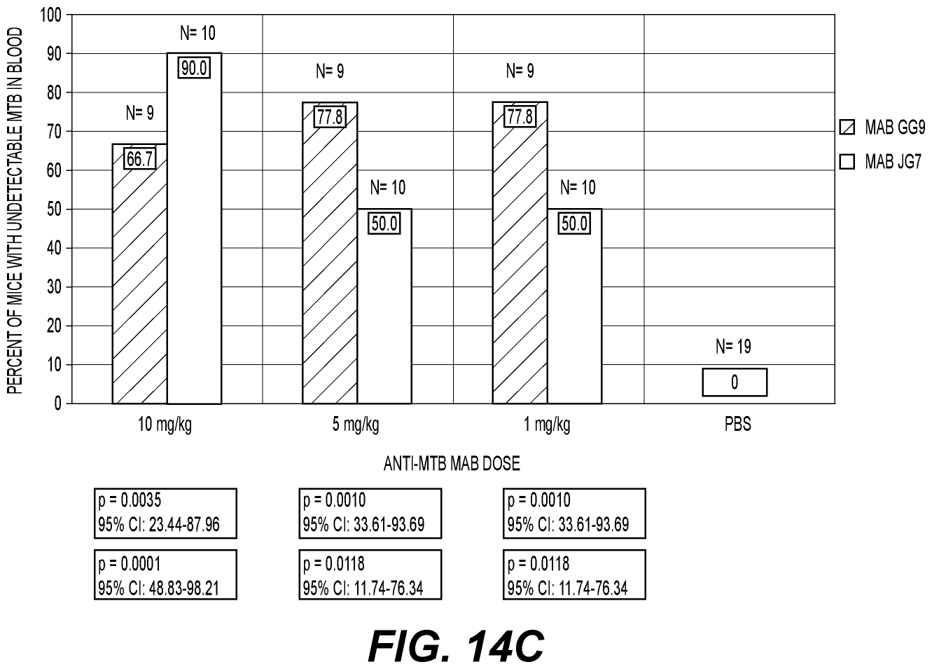

As shown in FIGS. 14A-C, using gPCR, rapid clearance of Mycobacterium tuberculosis (MTB) in blood was observed in all groups from the in vivo study with N=76 ICR mice. While MAB GG9 (FIG. 14A) significantly enhanced blood clearance at 24 hours post challenge (1 10 mg/kg p=0.0021, 10 mg/kg p=0.0013), MAB JC17 (FIG. 14B) significantly enhanced clearance at all time points (0.25, 4 and 24 hours) and at one or more doses. FIG. 14C shows the percentage of mice with undetectable levels of MTB in blood according to qPCR. Statistical significance determined by comparison of MAB-treated vs. PBS-treated blood samples from mice according to no detection (i.e. CT=40, qPCR.) was calculated using the Chi-squared test, with significance threshold set at p<0.05 and 95% confidence intervals shown. In addition, the MABs promoted rapid clearance of MTB from the blood of mice given as little as 1 mg/kg (FIG. 14A, 14B, 14C).

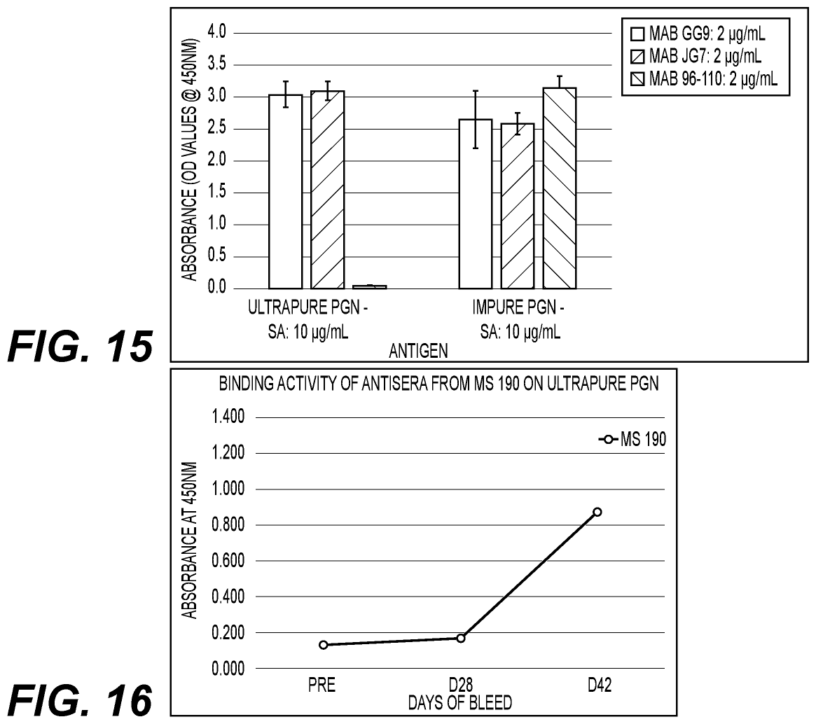

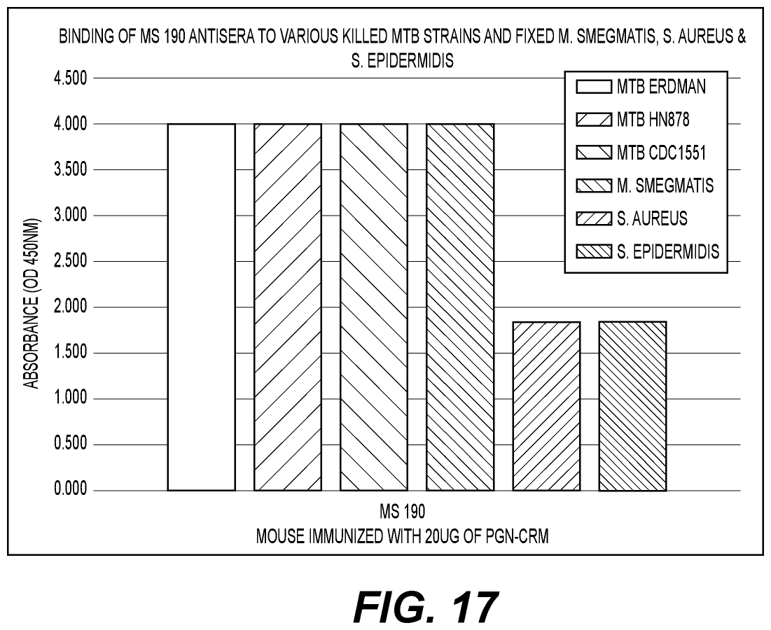

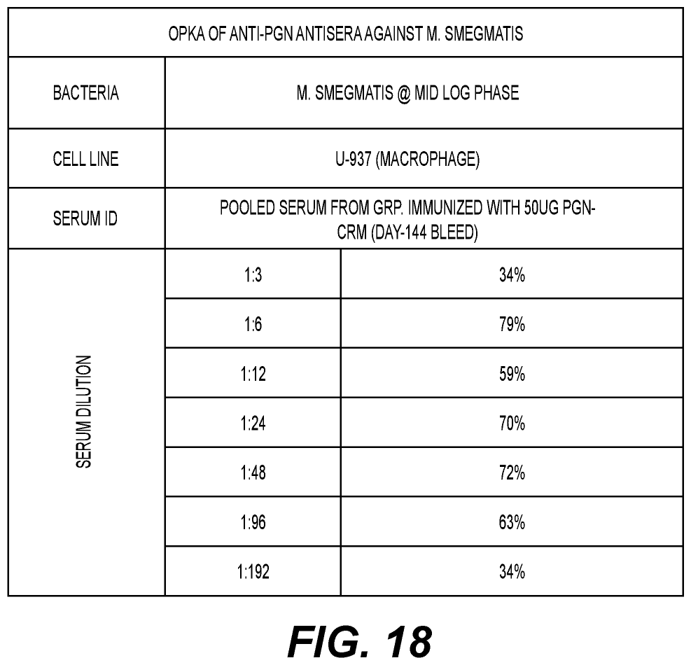

As shown in FIG. 15, MABs JG7 and GG9 and anti-LTA MAB (96-110) were analyzed for binding to a cell wall mixture and Ultrapure PGN, both from Staphylococcus aureus. Compared to a control MAB 96-110 directed against LTA that only bound to impure cell wall mixture containing components including LTA and PGN, MABs JG7 and GG9 bound to both cell wall mixture and ultrapure PGN (that does not contain other cell wall components such as LTA). This strongly suggests that MABs JG7 and GG9 bind to an epitope on PGN. PGN-binding activity of MABs GG9 and JG7 was demonstrated to Ultrapure and Impure PGN, while anti-LTA MAB 96-110 only bound the Impure PGN. MABs JG7 and GG9 are IgG1 and both MABs bound to ultra-pure peptidoglycan (PGN) (FIG. 15). Mice were subsequently immunized with CRM-conjugated PGN, and serum antibodies were induced that also reacted broadly across gram-positive bacteria and MTB. Moreover, the mice produced serum antibodies that bound to PGN and fixed bacteria. Mouse 190 (MS 190) serum antibodies showed good binding to ultrapure PGN (d42) (FIG. 16), bound broadly to various gram-positive bacteria (FIG. 17), and enhanced OPKA of M. smegmatis (FIG. 18). Mouse 190 (MS 190) with anti-PGN serum antibodies that also bound broadly to bacteria and enhanced OPKA was selected for hybridoma production (FIGS. 16-18).

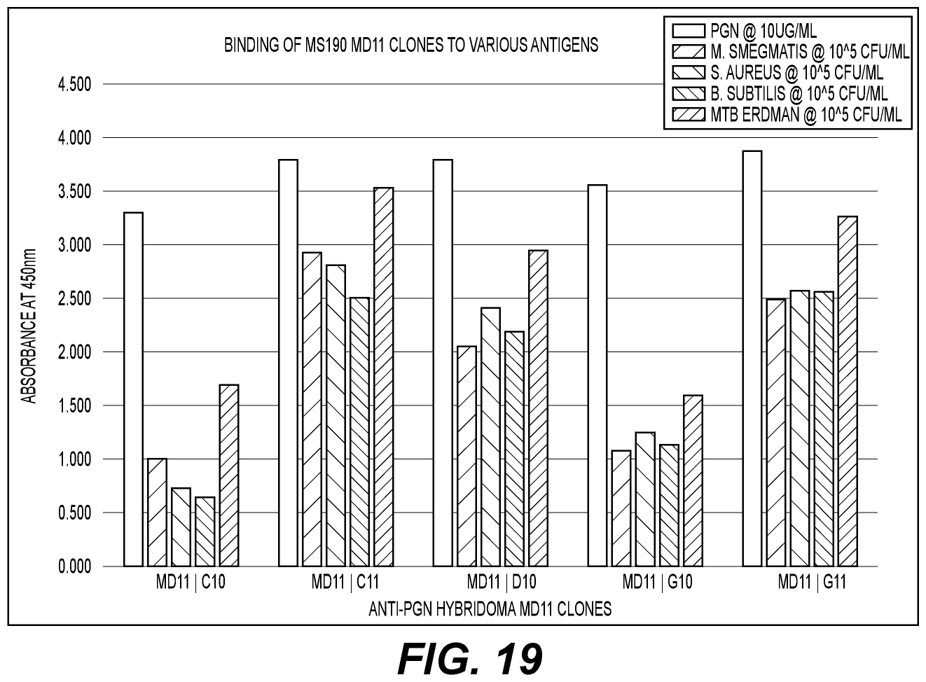

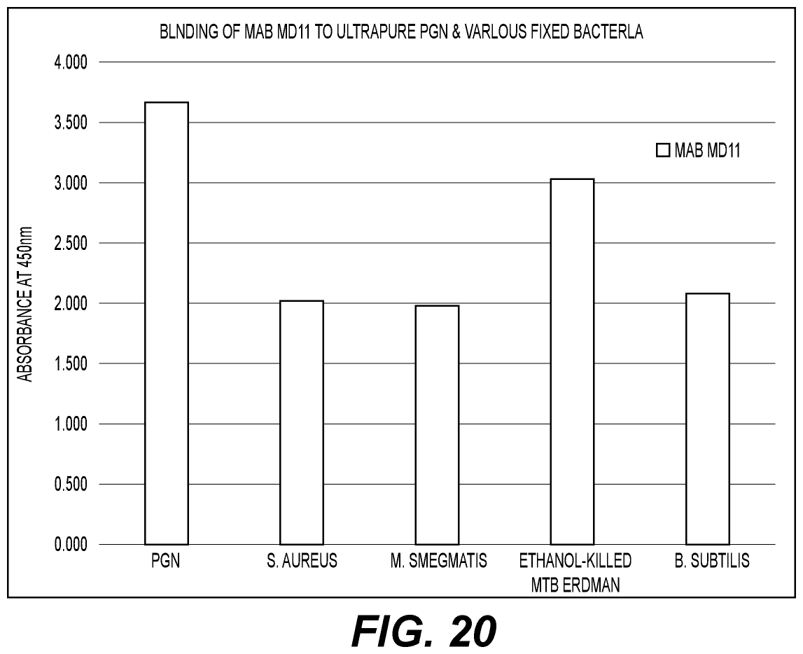

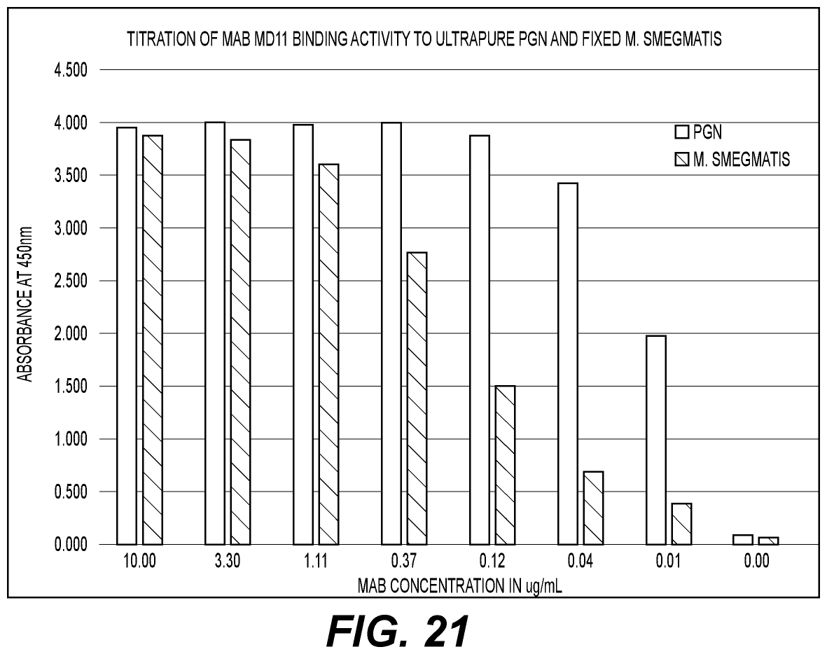

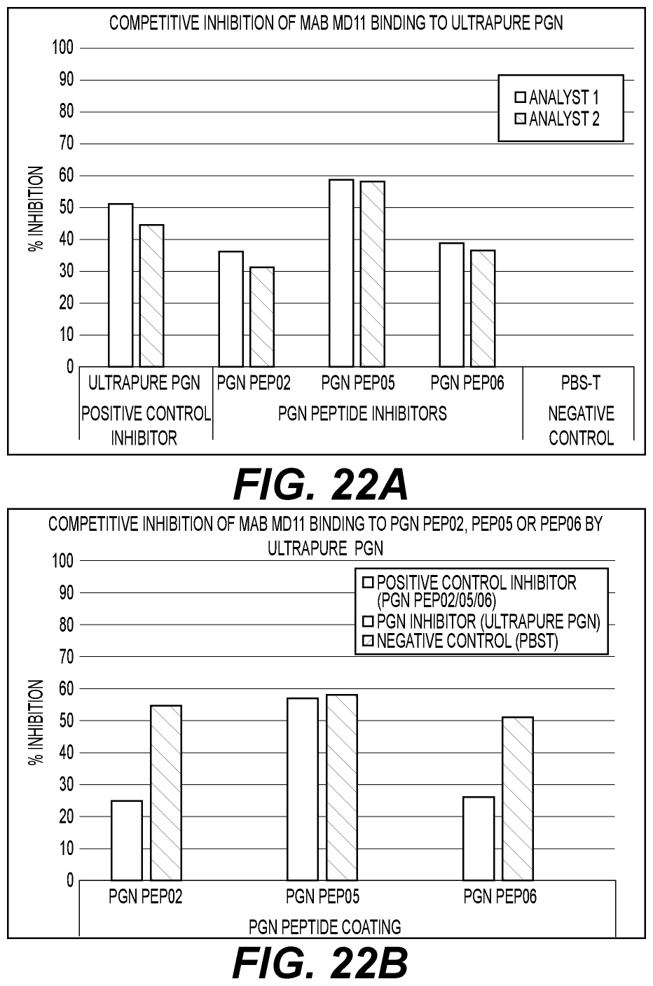

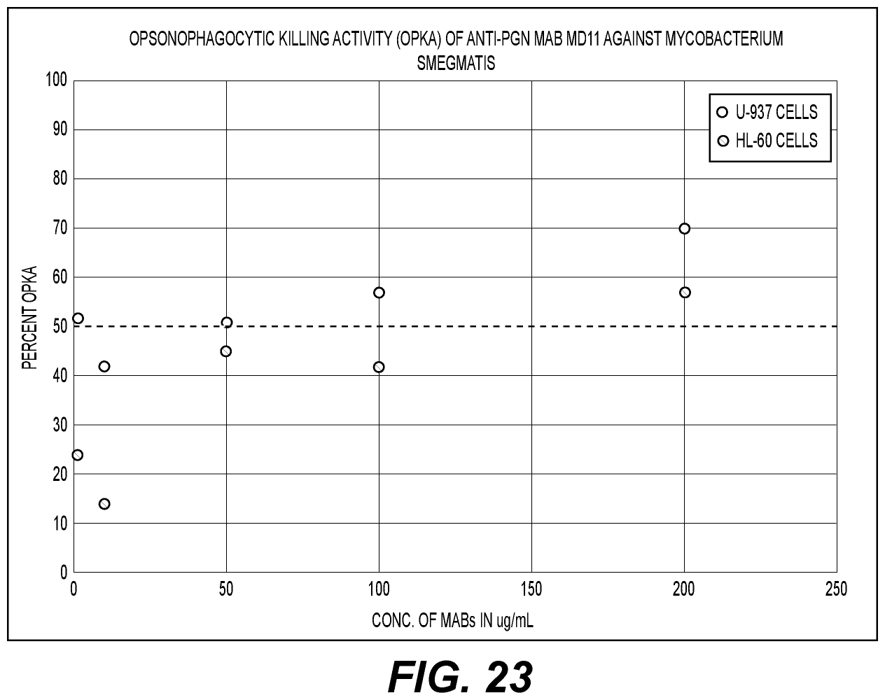

MS190 Hybridoma clone MD11 is an IgG2 MAB that hinds across multiple bacteria and ultra-pure PGN (FIGS. 19 and 21) which was identified from the hybridomas that were produced (FIG. 20). MAB MD11 showed binding activity to Peptidoglycan, killed MTB, and various strains of gram-positive bacteria (FIGS. 20 and 21). Conjugated PGN immunization induced broadly reactive antibodies to bacteria. As shown in FIGS. 22A and 22B, competitive inhibition of MAB MD11 binding to ultrapure PGN by small synthetic PGN peptides (FIG. 22A). Competitive inhibition of MAB MD11 binding to the small synthetic PGN peptides by Ultrapure PGN (FIG. 22B). In addition, MD11 bound to SMEG (FIG. 21) and promoted opsonophagocytic killing of SMEG and Staphylococci (>50% OPKA) using macrophages (U-937 cell line) and polymorphonuclear cells (PMNs; HL60 granulocytes), respectively (FIGS. 23 and 24).

### Example 6 Peptidoglycan Peptides and Composite Antigens

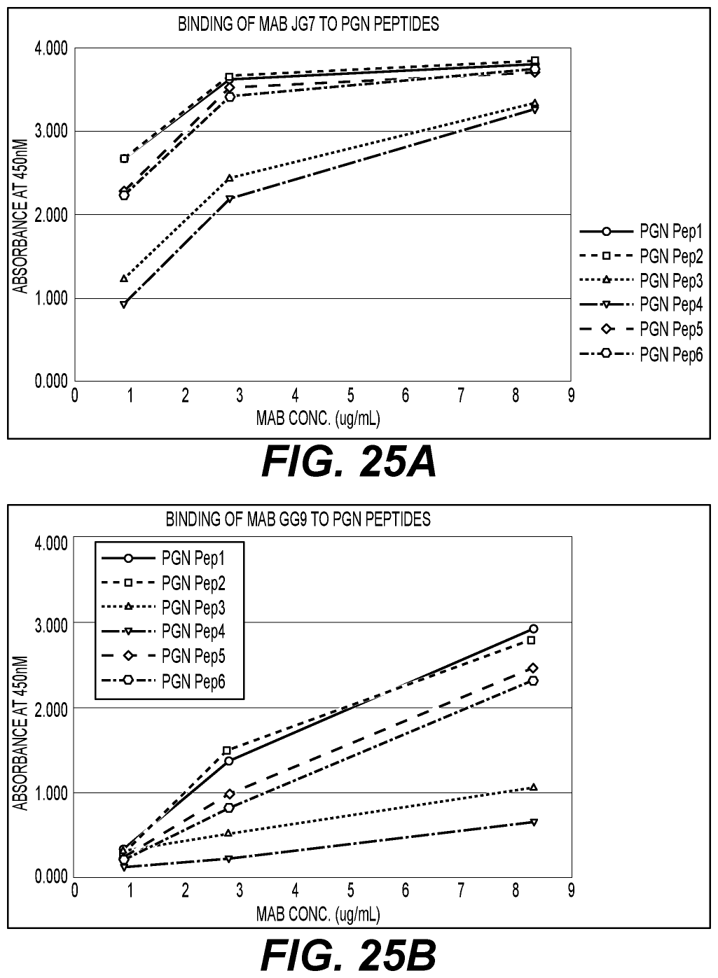

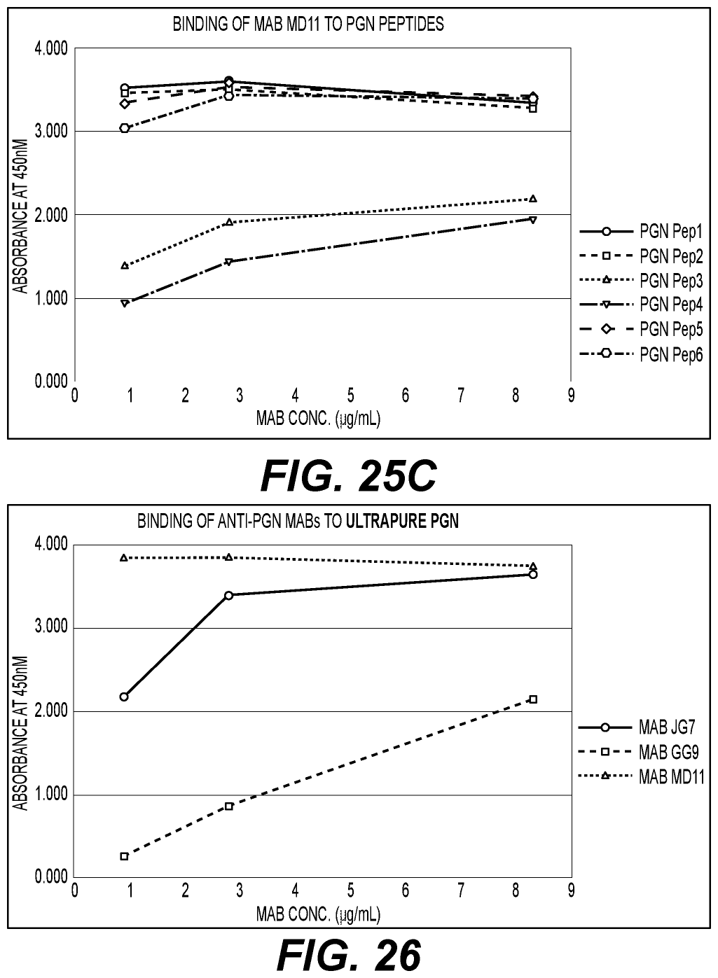

MABS JG7, GG9 and MD11 were analyzed for binding to small, synthesized peptides (FIGS. 25A, 25B and 25C) and to ultra-pure PGN (FIG. 26). MABs JG7 and GG9 are from mice immunized with ethanol killed MTB and MAB MD11 from a mouse immunized with CRM-conjugated PGN. Each of the MABs bound to all the small individual peptides (Table 1) and to PGN, but the binding patterns across the peptides were different.

### Example 7 Monoclonal Antibodies to Composite Peptides

Monoclonal antibodies (MABs) were developed against Mycobacterium tuberculosis Alpha Crystallin Heat Shock Protein. MAB LD7 (IgG2a) was derived from BALB/c MS 1435 immunized subcutaneously with TB Pep01 (Conserved Alpha Crystallin HSP), with Freund's adjuvant. MAB CA6 (IgG2b) was derived from BALB/c MS 1435 immunized subcutaneously with TB Pep01 (Conserved Alpha Crystallin HSP), with Freund's adjuvant.

PGN epitopes shown in Table 1 can be mixed and matched in varied combinations such as with or without a T cell epitope, to produce composite peptides and mixtures that could be formulated with adjuvants as MTB or Staph/Gram positive bacterial vaccines.

TABLE 4
MTB, LAM, and Staphylococcus LTA Peptide Sequences

| SEQ | | | | |
|---|---|---|---|---|
| ID | Peptide | | | |
| NO | number | Peptide ID | Peptide Sequence | Description |
| 140 | TB | LVD-PSEQ-A-TB | SEFAYGSFVRTVSLPVGADE | Conserved MTB |
| | Pep01 | Pep 01 | | Alpha Crystallin HSP |
| | | | | Epitope |
| 141 | TB | LVD-PSEQ-A-TB | SEFAYGSFVRTVSLPVGADE | Conserved MTB |
| | Pep02 | Pep 02 | GNLFIAPWGVIHHPHYEECSCY | Alpha Crystallin HSP |
| | | | | Epitope and 2 |
| | | | | conserved influenza |
| | | | | HA epitopes and 1 |
| | | | | conserved NA |
| | | | | Epitope |
| 142 | LAM | LVD-PSEQ-A- | HSFKWLDSPRLR | Conserved MTB |
| | Pep01 | LAM Pep 01 | | Lipoarabinomanin |
| | | | | Mimetope |
| 143 | LAM | LVD-PSEQ-A- | ISLTEWSMWYRH | Conserved MTB |
| | Pep02 | LAM Pep 02 | | Lipoarabinomanin |
| | | | | Mimetope |
| 144 | LTA | LVD-PSEQ-A- | WRMYFSHRHAHLRSP | LTA Epitope |
| | Pep01 | LTA Pep 01 | | |
| 145 | LTA | LVD-PSEQ-A- | WHWRHRIPLQLAAGR | LTA Epitope |
| | Pep02 | LTA Pep 02 | | |

SEQ ID No. 146: GNLFIAPWGVIHHPHYEECSCY = composite influenza peptide comprising HA and NA epitopes
SEQ ID No. 147: SEFAYGSFMRSVTLPPGADE = M. smegmatis peptide sequence

MTB, LAM and Staphylococcus LTA epitopes shown in Table 4 can be mixed and matched in combinations such as with or without a T cell epitope, to produce composite peptides and mixtures that could be formulated with adjuvants as MTB or Staph/Gram positive bacterial vaccines.

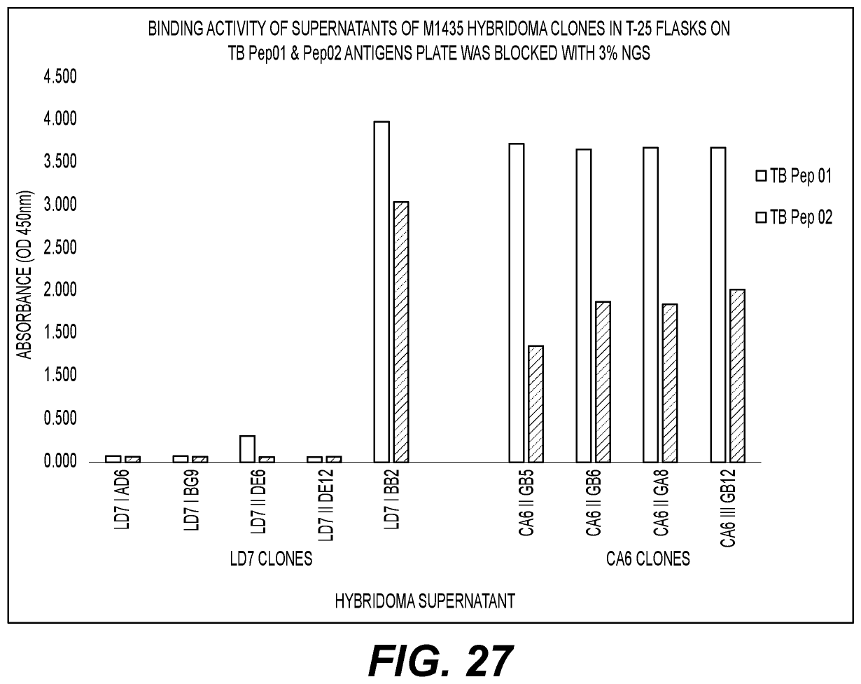

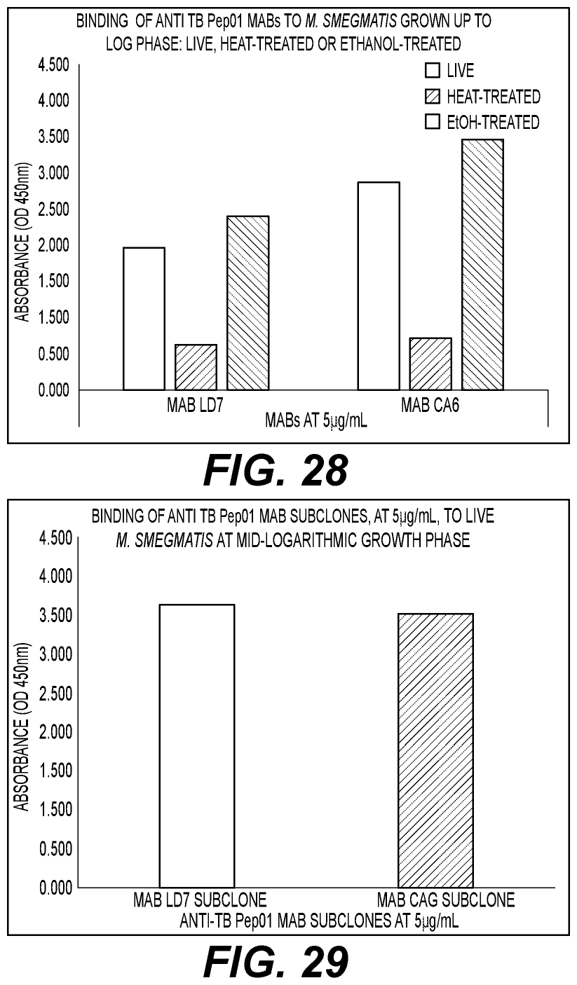

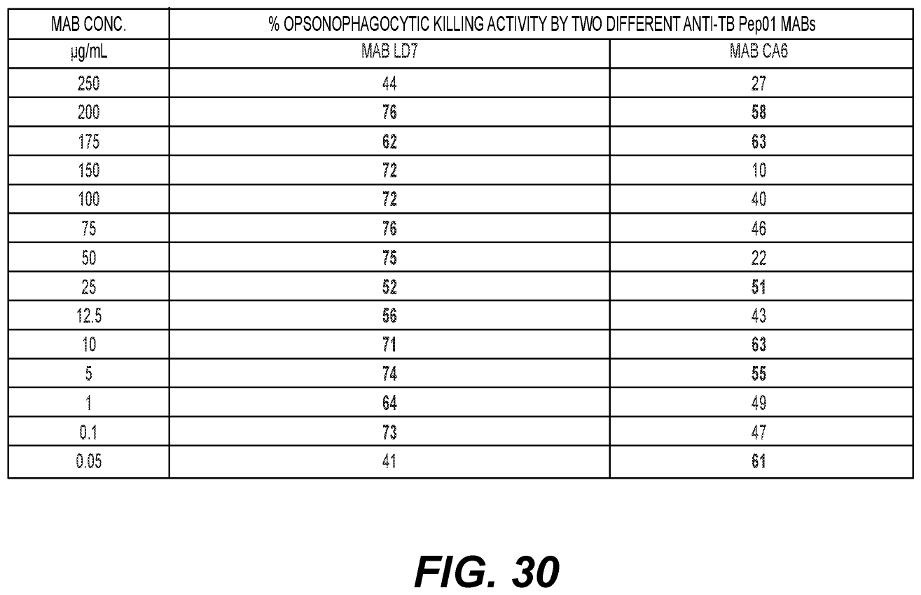

Binding activities of supernatants from hybridomas LD7 and CA6 to TB Pep01 and TB Pep02 at 1 μg/mL are shown in FIG. 27, to live Mycobacterium smegmatis (SMEG) in mid-log phase, live, heat-treated or ethanol-treated SMEG are shown in FIG. 28, and to live Mycobacterium smegmatis (SMEG) as demonstrated in a Live Bacteria ELISA is shown in FIG. 29. MABs LD7 and CA6 showed highly specific binding to the alpha crystallin HSP (TB Pep01) and promoted opsonophagocytic killing of M. smegmatis (SMEG). Enhanced OPKA of MABs LD7 and CA6 against Mycobacterium smegmatis (SMEG) using U-937 macrophages. Peak OPKA for LD7 was 76% and for CA6 was 63% (see FIG. 30). MABs were purified from hybridoma subclones and OD values (450 nM) for growth media without antibody (negative control) range between 0.046-0.060.

There is 80% homology (16 out of 20 amino acids) of HSP20 between M. tuberculosis (SEQ ID NO 140; SEFAYGSFVRTVSLPVGADE) and M. smegmatis (SEQ ID NO 147; SEFAYGSFMRSVTLPPGADE).

Mouse 1435 immunized with a conserved MTB alpha clystallin heat shock protein epitope developed serum antibodies that bound to a small synthesized alpha crystallin HSP peptide (TB Pep01). MAB LD7 (IgG2a) and MAB CA6 (IgG2b) that were subsequently produced from MS 1435 hound broadly to TB Pep01, TB Pep02 (composite peptide that constitutes TB Pep01, two conserved influenza hemaggiutinin epitopes, and one conserved neuraminidase epitope), and M. smegmatis (FIG. 27-30). In addition, these MABs showed enhanced OPKA (>50%) against M. smegmatis (FIG. 30).

The HSP epitope elicited strong humoral responses in mice, with high serum antibody titers and subsequently generated two MABs—LD7 and CA6 (IgG2a and IgG2b isotypes, respectively). These MABs bound strongly to the HSP epitope (OD450 nm of 3.0-3.5) but had low binding activity to fixed mycobacteria (OD450 nm<0.25). Notably, MABs LD7 and CA6 showed significantly increased binding activity to live SMEG, compared to fixed SMEG, and demonstrated significant OPKA against SMEG at both low (0.1 μm/mL) and high (200 μm/mL) antibody concentrations.

The small conserved synthetic HSP epitope induced a robust humoral response in mice and generated two MABs that recognized live SMEG and demonstrated significant OPKA against SMEG at MAB concentrations as low as 0.1 μm/mL. Immunization with this small conserved synthetic HSP epitope generates opsonic antibody responses against mycobacteria and provide important strategies for TB vaccines and therapeutics.

### Example 8 Composite Peptide Vaccines for Influenza and Other Viruses

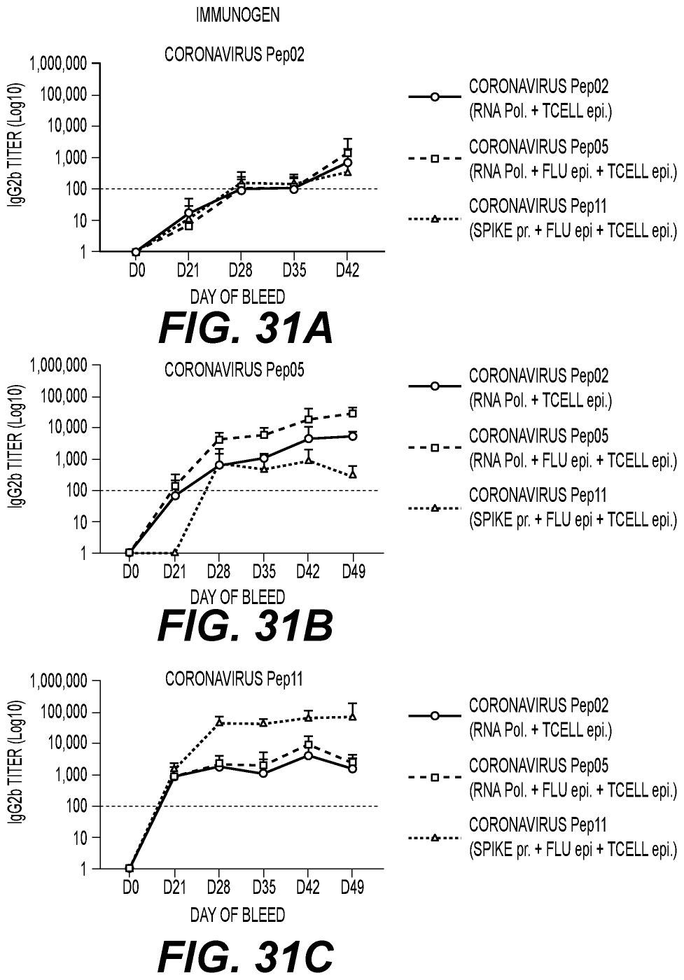

An influenza composite vaccine comprising small conserved epitopes such as HA, NA, or matrix peptide sequences induce broadly neutralizing antibodies across Group 1 and 2 Influenza A viruses. Combining one or more of these peptides with one or more small, conserved peptide sequences from two or more viruses (such as influenza and coronavirus) provides a prototype composite virus peptide vaccine that broadens the vaccine's prevention or treatment capabilities to include more than one virus (FIGS. 31-35). Combined influenza and coronavirus composite peptide vaccine antigens were synthesized and included the conserved influenza matrix and NA peptides plus the conserved coronavirus polymerase peptide (Cor Pep 05), or spike protein conserved sequence (Cor Pep 11) and a T cell epitope sequence (Table 5). The polymerase conserved epitope was also sequenced alone with the T cell epitope (Cor Pep 02).

TABLE 5
Composite Peptide Antigens for Influenza and Other Viruses

| SEQ | | | |
|---|---|---|---|
| ID | Peptide | | |
| NO | number | Peptide ID | Peptide Sequence |
| 156 | Coronavirus | LVD-PSEQ-A-Coronavirus Pep06 | WDYPKCDRATEVETPIRNEHYEECS |
| | Pep05 | | CYQYIKANSKFIGITE |
| 157 | Flu Pep03 | LVD-PSEQ-A-Flu Pep03 | GNLFIAP |
| 158 | Flu Pep06 | LVD-PSEQ-A-Flu Pep06 | WGVIHHP |
| 159 | Flu Pep10 | LVD-PSEQ-A-Flu Pep10 | HYEECSCY |
| 160 | Cor Pep13 | LVD-PSEQ-A-Coronavirus Pep13 | YFPLQSYGFQPTNGVGYQPYR |
| 161 | Cor Pep14 | LVD-PSEQ-A-Coronavirus Pep14 | YFPLQSYGFQPTNGVGYQPYRQYIK |
| | | | ANSKFIGITE |
| 163 | Cor Pep15 | LVD-PSEQ-A-Coronavirus Pep15 | YQAGSTPCNGVEGENCYFPLQ |
| 162 | Cor Pep16 | LVD-PSEQ-A-Coronavirus Pep16 | YQAGSTPCNGVEGENCYFPLQYIKA |
| | | | NSKFIGITE |
| 164 | Flu Pep52 | LVD-PSEQ-A-Flu Pep52 | ETPIRNE |
| 165 | Flu Pep53 | LVD-PSEQ-A-Flu Pep53 | TEVETPIRNE |
| 166 | Flu Pep57 | LVD-PSEQ-A-Flu Pep57 | SLLTEVETPIRNEWGLLTEVETPIR |
| 167 | Cor Pep11 | LVD-PSEQ-A-Coronavirus Pep11 | ENQKLIANTEVETPIRNEHYEECSCY |
| | | | QYIKANSKFIGITE |
| 168 | CorPep01 | LVD-PSEQ-A-Cor Pep 01 | WDYPKCDRA |
| 169 | Cor Pep02 | LVD-PSEQ-A-Cor Pep 02 | WDYPKCDRAQYIKANSKFIGITE |
| 170 | Cor Pep05 | LVD-PSEQ-A-Cor Pep 05 | WDYPKCDRATEVETPIRNEHYEECS |
| | | | CYQYIKANSKFIGITE |
| 171 | Cor Pep09 | LVD-PSEQ-A-Cor Pep 09 | ENQKLIAN |
| 172 | Cor Pep11 | LVD-PSEQ-A-Cor Pep 11 | ENQKLIANTEVETPIRNEHYEECSCY |
| | | | QYIKANSKFIGITE |

Description of sequences listed in Table 5.
156. Coronavirus RNA polmerase and influenza matrix and neuraminidase (NA) peptides, with a T cell epitope.
157-159. Influenza peptides-3 (Hemagglutinin, HA) 6 (HA), and 10 (NA).
160-163. Coronavirus peptides with and without a T cell epitope.
164-165. Influenza peptides-52 and 53.
166. Influenza peptide 57.
167. Coronavirus spike protein epitope and influenza matrix and NA peptides with a T cell epitope.
168. RNA polymerase region, non-spike.
169. Conserved regions from the RNA polymerase, Tetanus T-cell epitope.
170. Conserved regions from the RNA polymerase + Flu Pep53 (M2), Flu Pep10, Tetanus T-cell epitope.
171. Epitopes on spike protein.
172. Conserved SARS epitopes, Flu Pep53 (M2), Flu Pep10, Tetanus T-cell epitope.

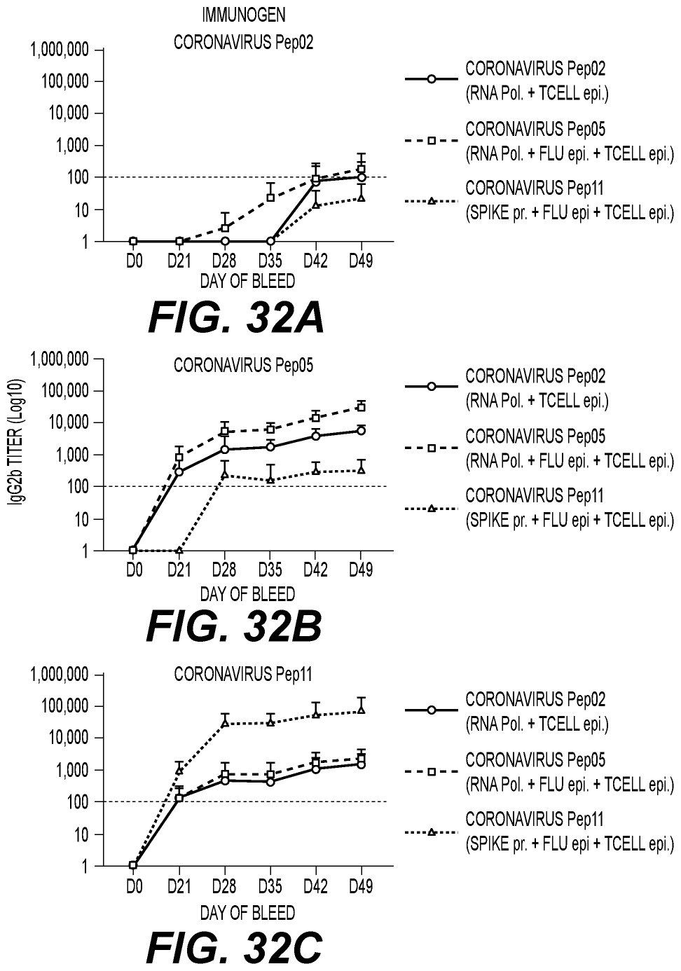

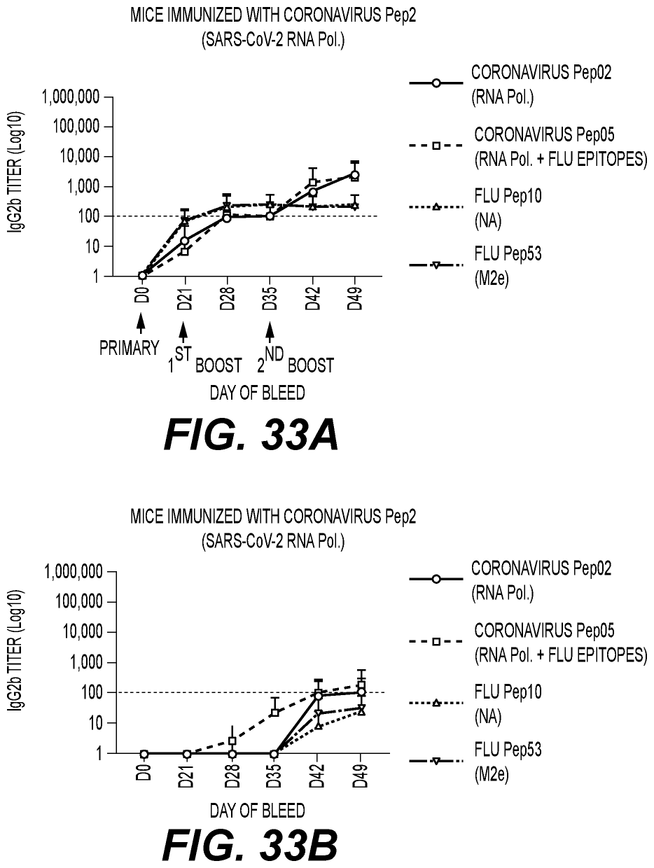

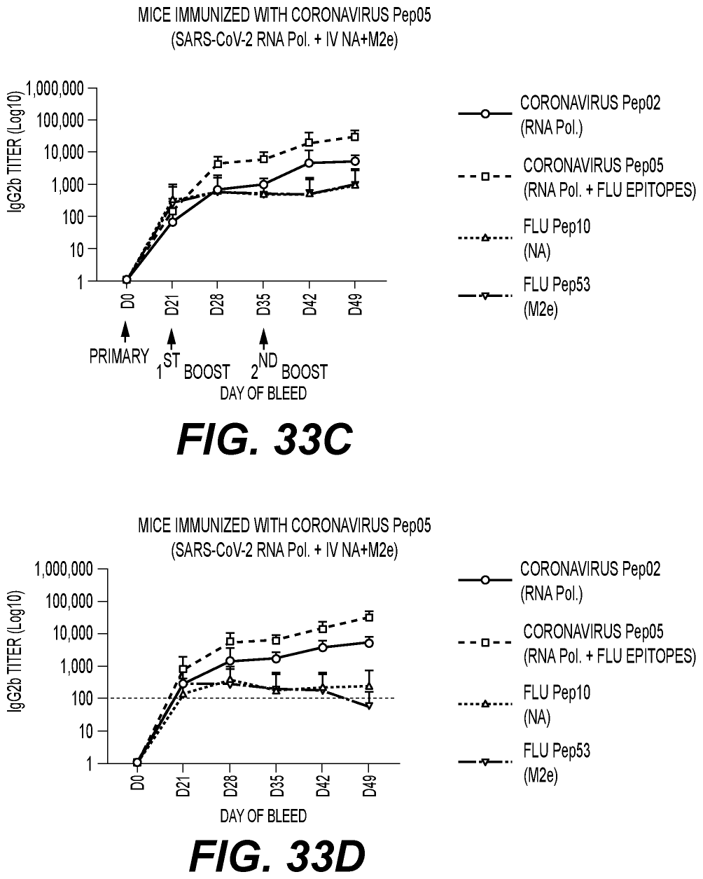

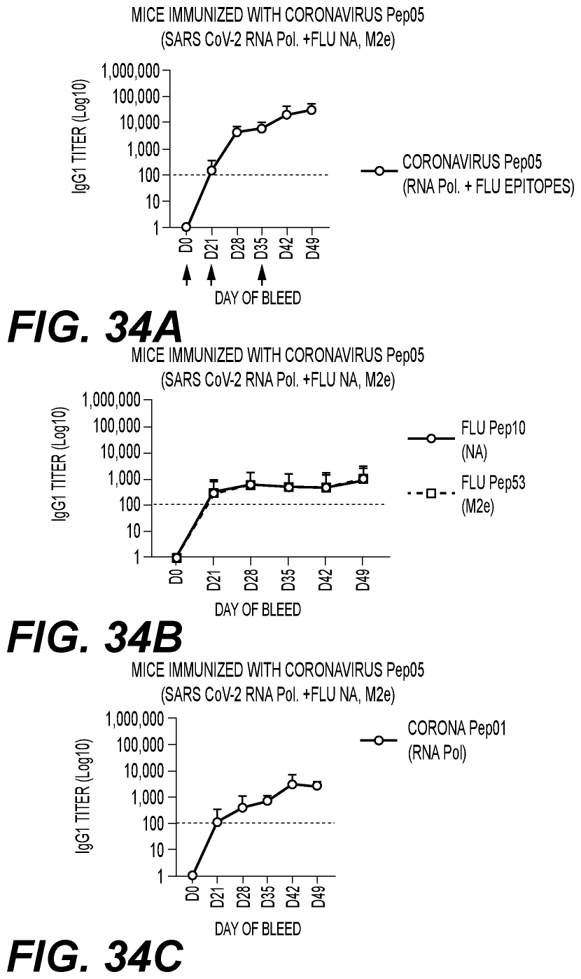

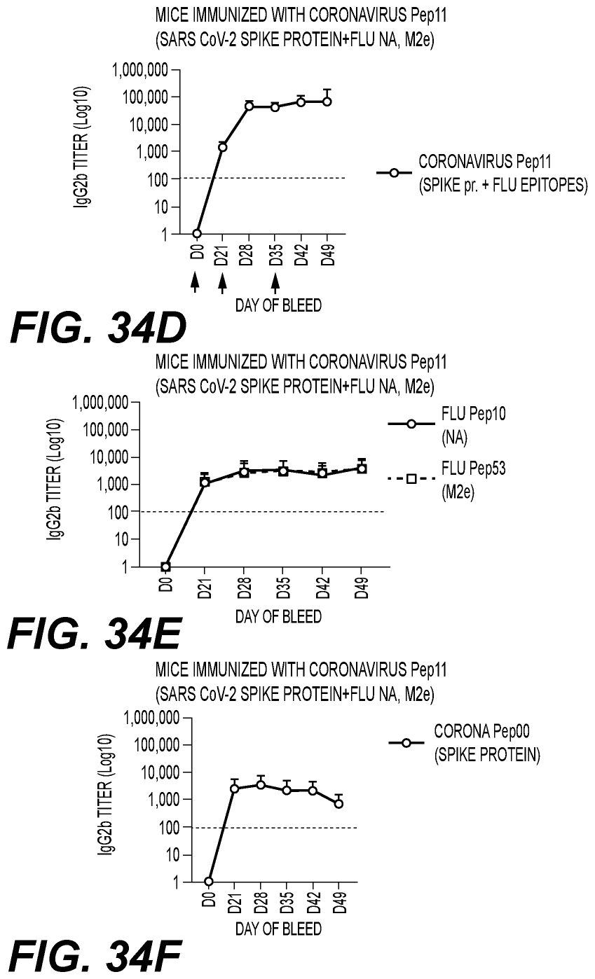

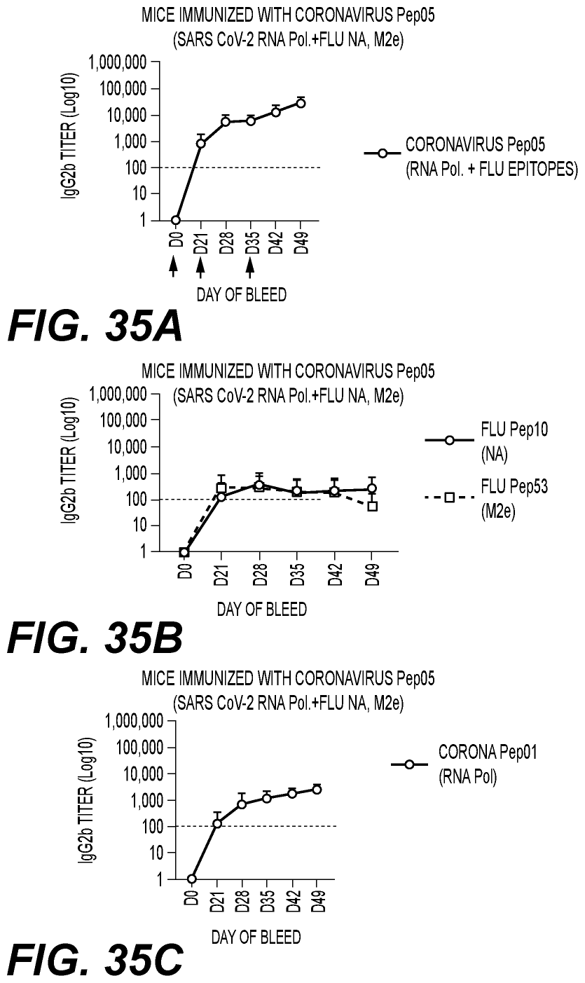

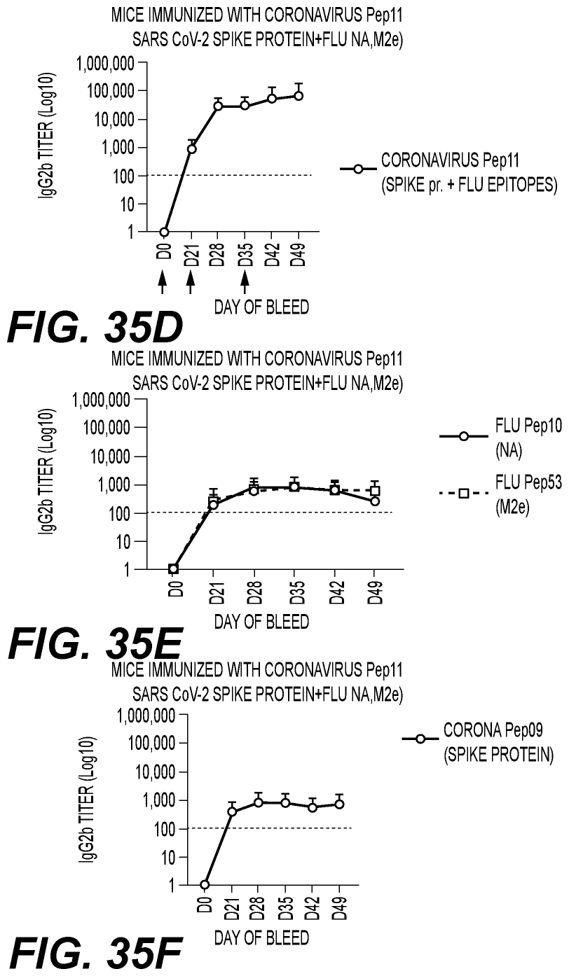

Mice were immunized with one, or more of these peptides formulated with ADDAVAX™ adjuvant and given by either subcutaneous (SQ) injection at a dose of 20 μg, or Intradermal (ID) injection at 1, 10, or 20 μg on days 0, 21 and 35. Robust serum IgG1 and IgG2b antibodies were induced to the conserved influenza and coronavirus epitopes and to whole coronavirus and influenza viruses. Serum antibody responses in mice immunized subcutaneously with 20 μg dose of Coronavirus Pep02 (FIG. 31A), Coronavirus Pep05 (FIG. 31B, or Coronavirus Pep11 (FIG. 31C, and booster immunizations given on Days 21 and 35. Profile of IgG1 antisera titers (FIGS. 31A-C) or IgG2b antisera titers (FIGS. 32A-C) to the immunogens are shown as Mean±SD (FIGS. 31A-C). Serum antibody responses in mice immunized with Coronavirus Pep02 (FIGS. 33A and B) and Coronavirus Pep05 (FIGS. 33C and D). IgG1 antisera titers to the composite coronavirus peptides are shown in FIGS. 33 A and C; and IgG2b titers to the same peptides are shown in FIGS. 33B and D. Serum antibody responses in mice immunized with Coronavirus Pep05 (FIG. 34A-C and FIGS. 35A-C) and Coronavirus Pep11 (FIG. 34D-F and FIGS. 35D-F). Profile of IgG1 or IgG2b antisera titers to the composite coronavirus peptides are shown in FIGS. 34A and D or FIGS. 35A and D; titers to influenza epitopes are indicated in FIGS. 34B and E or FIGS. 35B and E; and titers to individual coronavirus RNA polymerase and spike protein epitopes are demonstrated in FIGS. 34C and F or FIGS. 35C and F.

In addition, the antisera titers rose rapidly to the polymerase and spike coronavirus epitopes on the homologous composite peptide antigens and to the influenza epitopes on the composite antigens (FIGS. 31-35). The composite peptide antigens that included both coronavirus and influenza peptides with a T-cell epitope, provided a greater response than the composite peptide with the coronavirus polymerase epitope and a T-cell epitope. In addition, comparing the IgG responses to polymerase and spike protein epitopes showed dramatically different profiles with antibodies to polymerase steadily increasing over 49 days, while spike antibodies went up rapidly and either flattened or dropped between days 28 and 49 (FIGS. 34, 35). Antisera titers to the influenza epitopes increased rapidly and then leveled off after day 21.

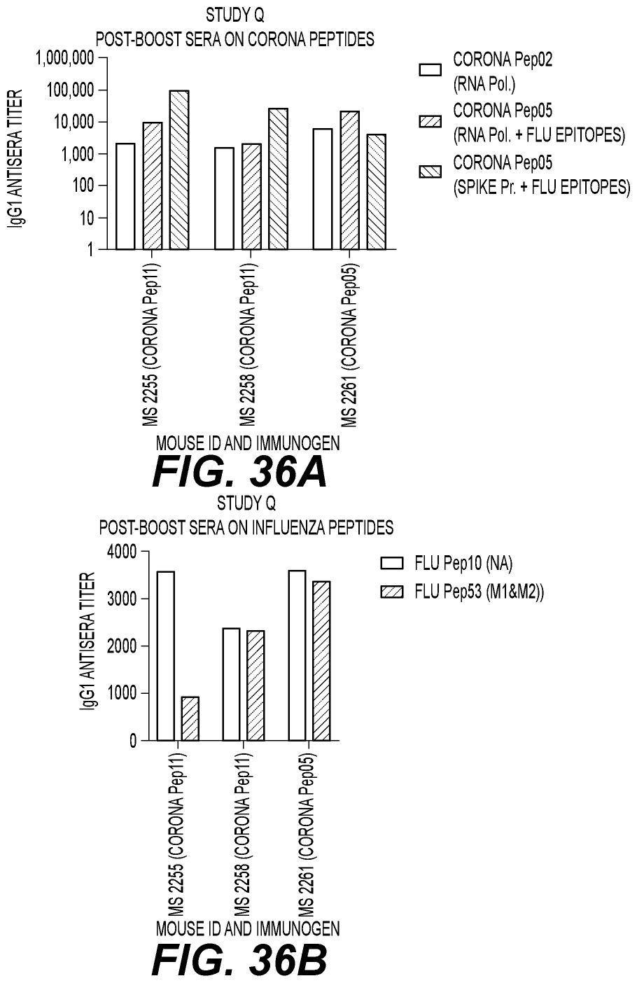

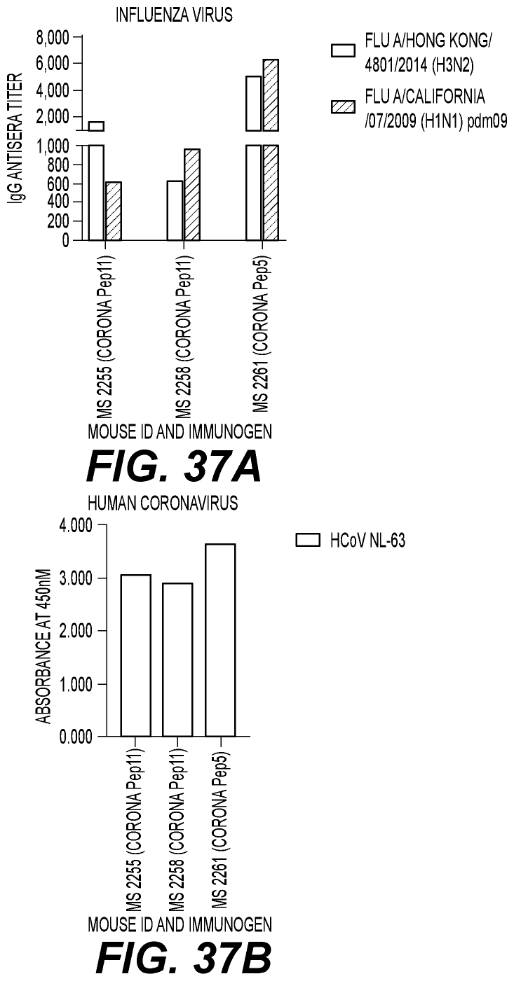

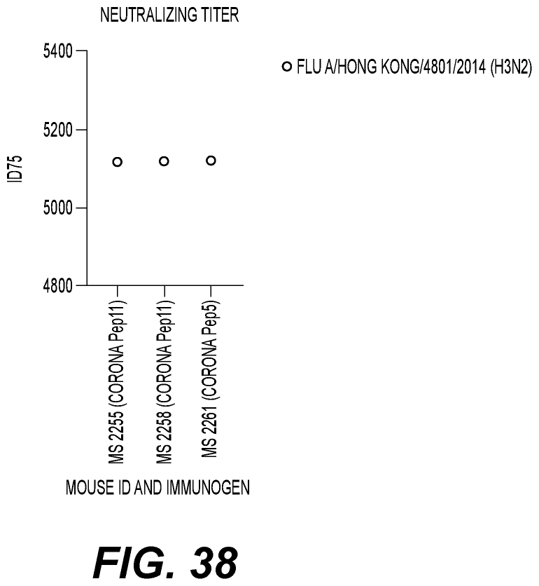

Serum antibody responses in select mice immunized subcutaneously with 20 μg dose of either Coronavirus Pep11 or Coronavirus Pep05 (FIG. 36A). One year post primary immunizations, the selected mice were given a boost and bled a week after. IgG1 antibody titers to coronavirus peptides (FIG. 36A) and influenza epitopes (FIG. 36B). Serum antibody responses in select mice immunized subcutaneously with 20 μg dose of either Coronavirus Pep11 or Coronavirus Pep05. One year post primary immunizations, the selected mice were given a boost and bled a week after. IgG antibody titers to influenza virus A (FIG. 37A) and IgG responses to human Coronavirus (FIG. 37B). Neutralizing titers in select mice immunized subcutaneously with 20 μg dose of either Coronavirus Pep11 or Coronavirus Pep05. One year post primary immunizations, the selected mice were given a boost and bled a week after. Neutralization of influenza A/Hong Kong (H3N2) (ID₇₅ values) (FIG. 38). Furthermore, the durability of the antibody responses were strong for both coronavirus and influenza peptides in the composite peptide antigens and for the antisera binding to influenza and coronavirus viruses one year after primary immunization (FIGS. 36-38).

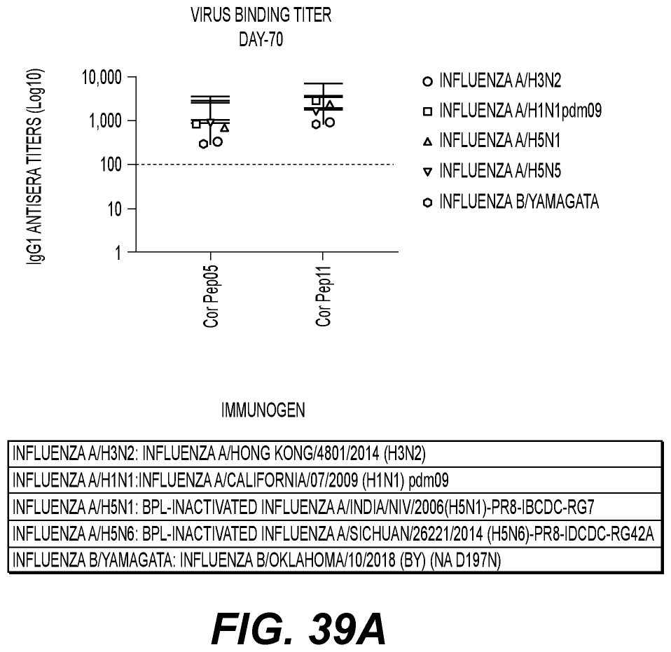

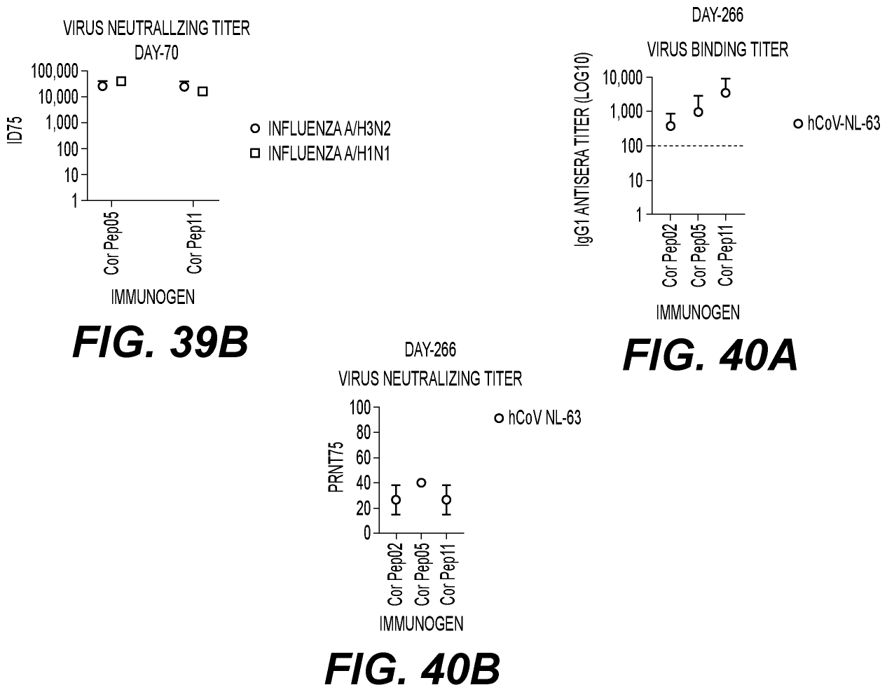

Also, 70 days after initial immunization, antisera bound across Groups 1 (H1N1) and 2 (H3N2) influenza A viruses and influenza B virus with strong neutralization (FIGS. 39A and B). IgG1 antisera titers (day 266) to human Coronavirus (hCoV) NL-63 are demonstrated in FIG. 40A. End-point neutralization titers based on 75% neutralization of hCoV NL-63 are shown as PRNT75 values in FIG. 40B.

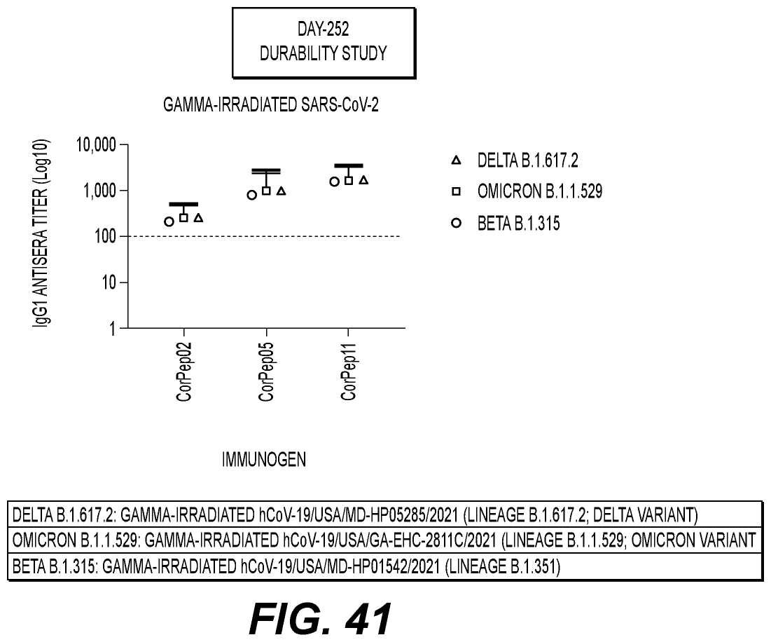

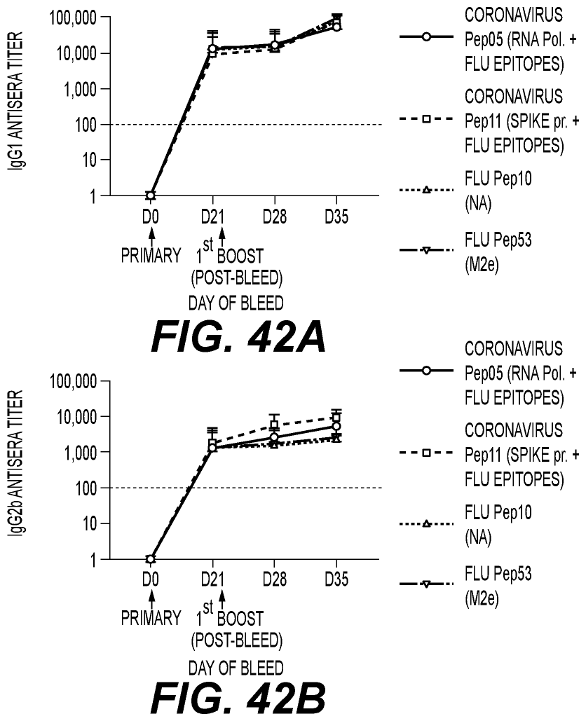

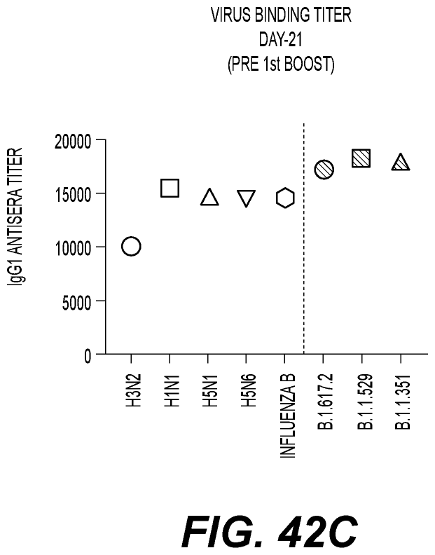

In addition, day 252 IgG1 antisera bound strongly across 3 variants of gamma-irradiated SARSCoV-2 variants re shown in mice immunized with Coronavirus Pep02, Coronavirus Pep05 and Coronavirus Pep11 (FIG. 41). Serum antibody responses were measured in mice immunized with a combination of Coronavirus Pep05 and Coronavirus Pep11. IgG1 and IgG2b antisera titers to the composite coronavirus peptides and influenza epitopes are shown in FIGS. 42A and B, respectively. Virus binding titers (IgG1) to various subtypes of influenza A and B and three variants of SARS-CoV-2 are demonstrated in FIG. 42C.

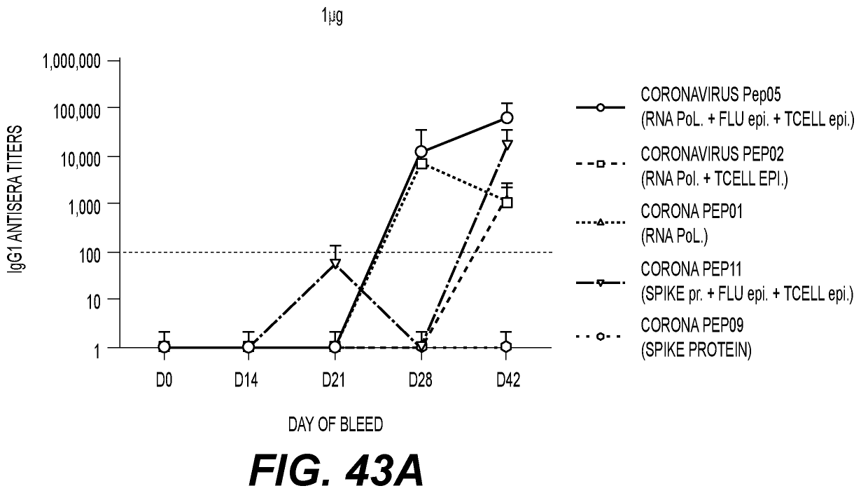

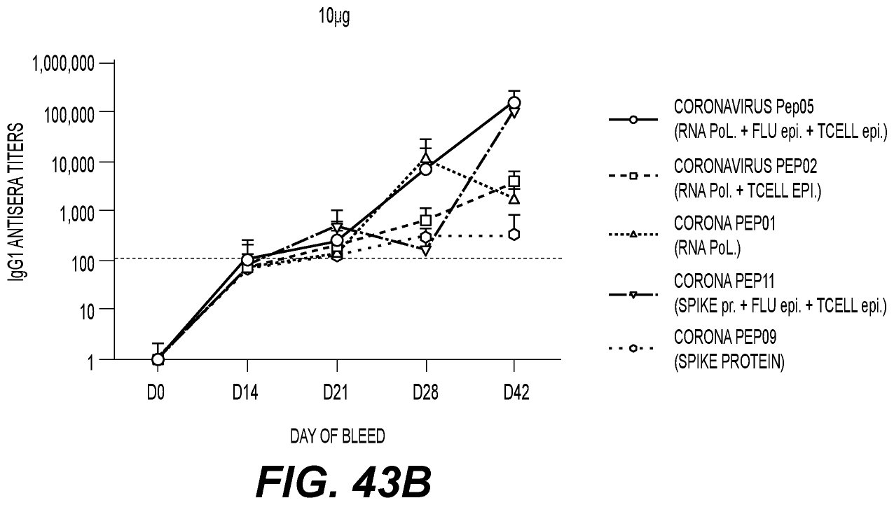

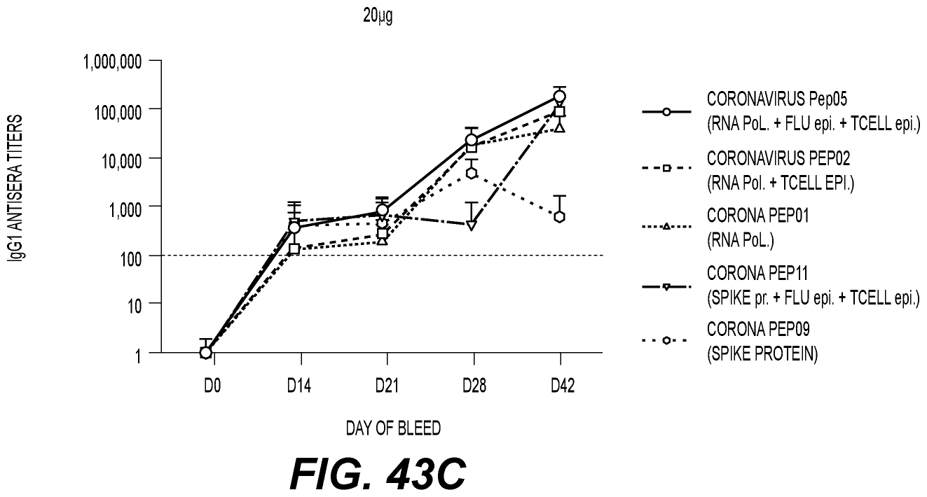

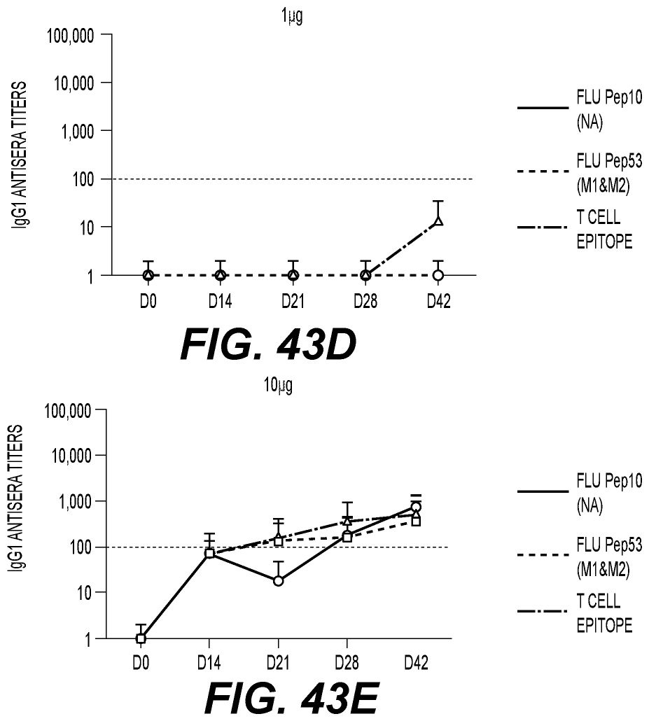

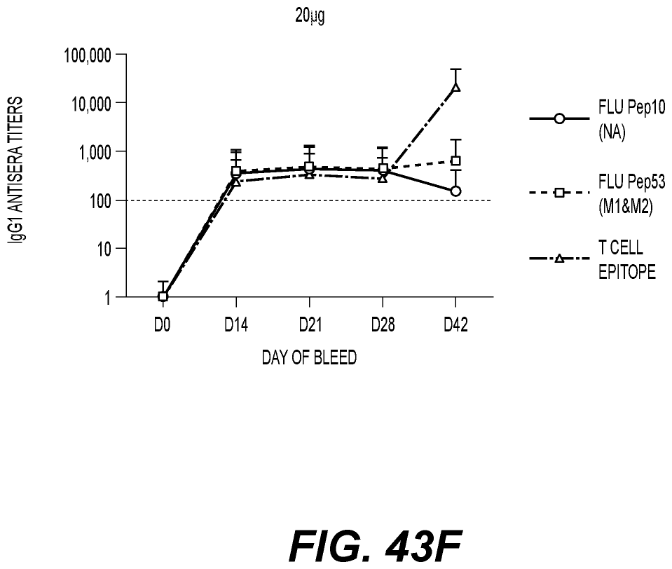

Serum antibody responses in mice immunized intradermally with 1 μg, 10 μg or 20 μg dose of a composite vaccine comprising of Coronavirus Pep11 and Coronavirus Pep05 and booster immunizations given on days 21 and 35. IgG1 antisera titers to the coronavirus peptides for each dose group are shown in FIGS. 43A-C, titers to influenza epitopes and universal T cell epitopes for each dose group are indicated in FIGS. 43D-F.

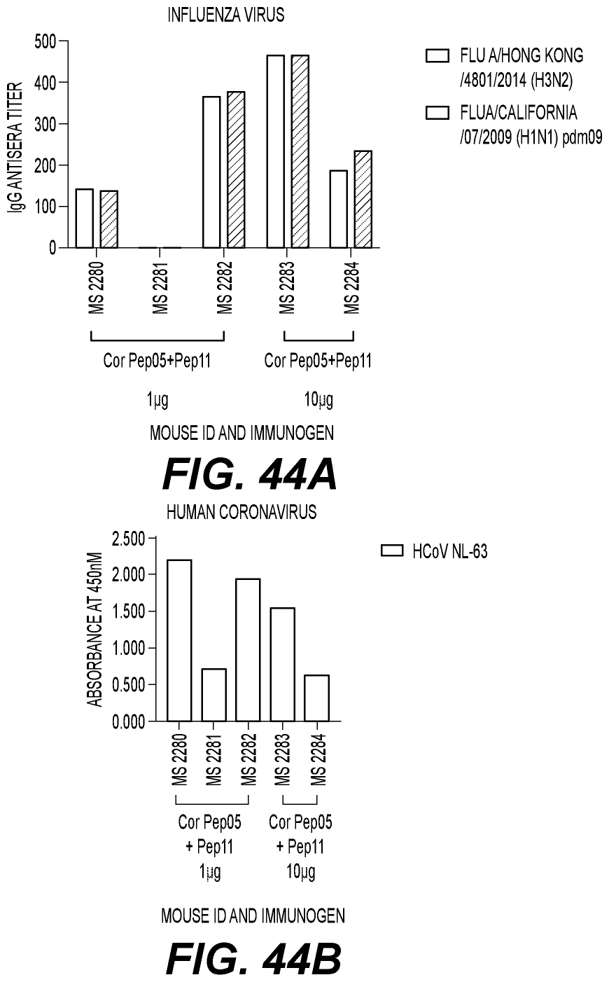

Serum antibody responses in mice immunized intradermally with 1 μg, 10 μg or 20 μg dose of a composite vaccine comprising of Coronavirus Pep11 and Coronavirus Pep05. One year post primary immunizations, the mice were given a boost and bled a week after. IgG antibody titers to influenza virus A (FIG. 44A) and IgG responses to human Coronavirus (FIG. 44B).

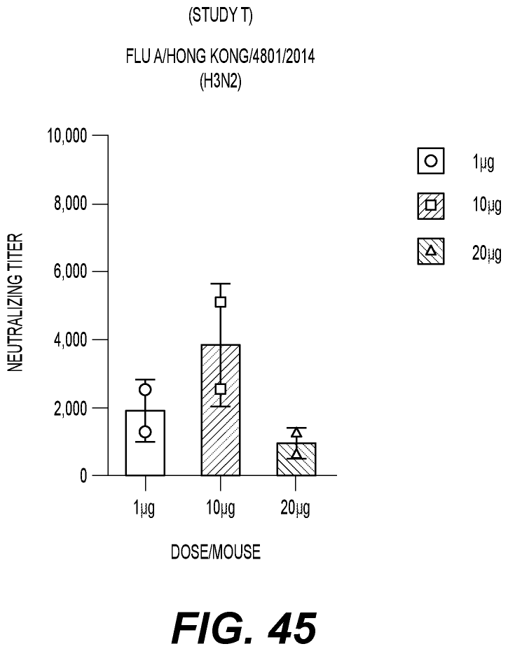

Neutralizing titers (day 56) in mice immunized intradermally with 1 μg, 10 μg or 20 μg dose of a composite vaccine comprising of Coronavirus Pep11 and Coronavirus Pep05. Neutralization of influenza A/Hong Kong (H3N2) (FIG. 45).

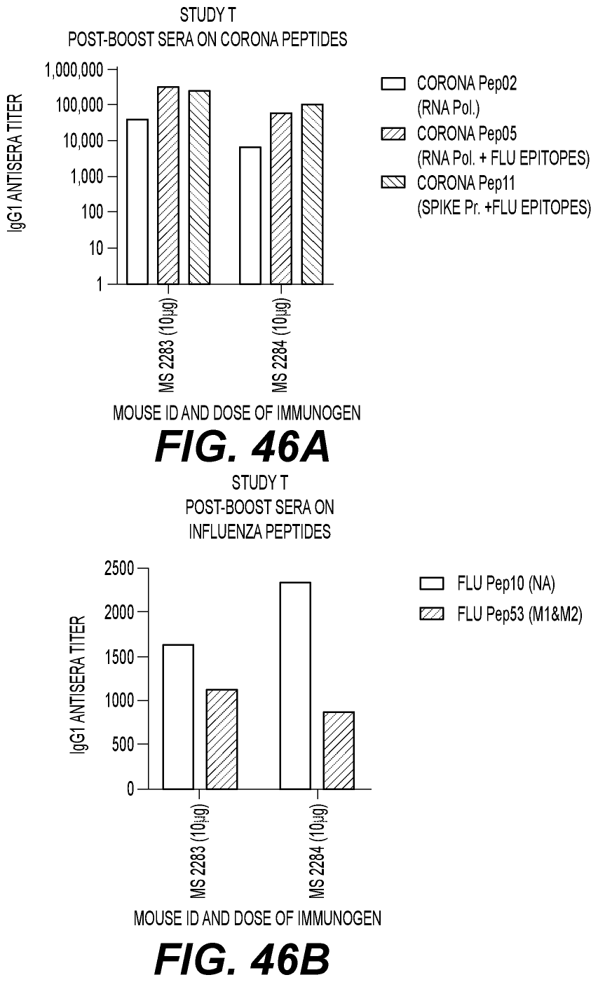

Serum antibody responses in select mice immunized intradermally with 10 μg of a composite vaccine comprising of Coronavirus Pep11 and Coronavirus Pep05. One year post primary immunizations, the mice were given a boost and bled a week after. IgG1 antibody titers to coronavirus peptides (FIG. 46A) and influenza epitopes (FIG. 46B).

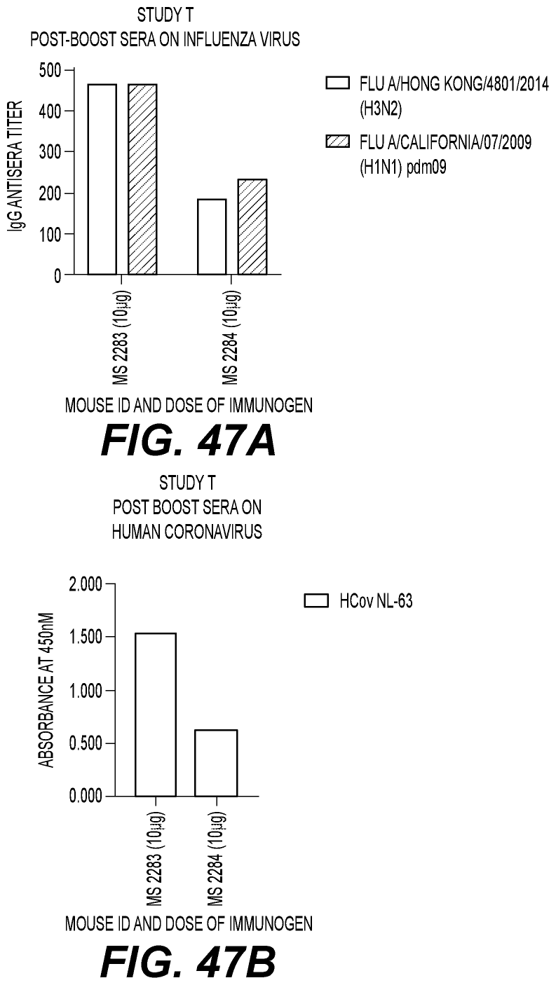

Serum antibody responses in select mice immunized intradermally with 10 μg of a composite vaccine comprising of Coronavirus Pep11 and Coronavirus Pep05. One year post primary immunizations, the mice were given a boost and bled a week after. IgG titers to influenza virus A (FIG. 47A) and IgG antibody responses to human Coronavirus (FIG. 47B).

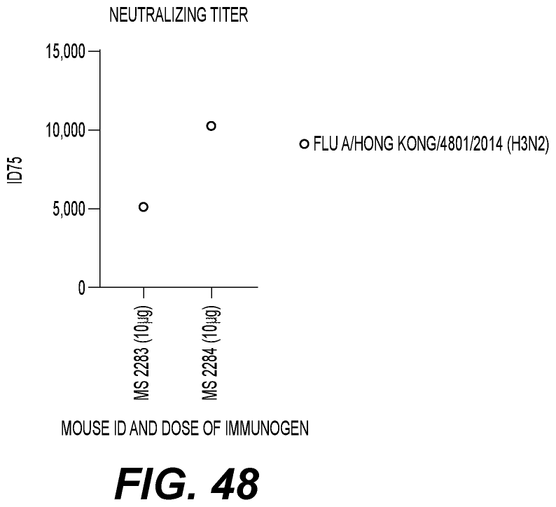

Neutralizing titers in select mice immunized intradermally with 10 μg of a composite vaccine comprising of Coronavirus Pep11 and Coronavirus Pep05 are shown in FIG. 48. Neutralization of influenza A/Hong Kong (H3N2) (ID₇₅ values) is shown one year post primary immunization.

These studies show that serum antibodies induced by both SQ and ID immunization bound to both live influenza and coronavirus and were strongly neutralizing (FIGS. 38-40, 45, and 48). In addition, mice were immunized with both coronavirus peptide vaccines Pep05 and Pep11 formulated together into a single vaccine (FIG. 42). The combined composite vaccine antigens induced an amazingly robust early response to coronavirus and influenza peptide epitopes and IgG1 bound at very high titers to influenza A strains and to influenza B as well SARSCoV-2 variants. These results are surprising and formulating composite peptide vaccine antigens together does not cause inhibition of epitope responses, but actually increases immune stimulation and may be related to increased number of immune cells activated. Further studies were done with different routes of administration and using different composite vaccine antigen doses. Both 1 and 10 μg doses induced serum IgG responses that were boosted one year after initial immunization (FIGS. 44-48). Robust antibody responses were seen with 1 and 10 μg doses and to include very strong influenza virus neutralization. These data demonstrate that the peptide composite vaccines can induce a robust immune response when given by various immunization routes.

Composite vaccine antigens that include HIV and malaria epitopes (SEQ ID Nos 214-218) provide Composite vaccines against important viral and parasitic pathogens. Composite peptide vaccine antigens provide efficiencies for vaccine production and delivery to populations around the world that often live in rural areas that lack medical infrastructure and require inexpensive vaccines that can provide broad immunity to key pathogens such as HIV and malaria, as well as influenza and tuberculosis.

Malaria and LPS, Lipooligosaccharide Epitopes
Malaria Epitope (CSP junctional region-
antibody blocks liver invasion)
SEQ ID No. 214: NPDPNANPNVDPNANGGGC
Lipooligosaccharide mimotopes
(nontypeable H. influenza)
SEQ ID No. 215: NMMRFTSQPPNNNMMNYIMDPRTH
E. coli and Salmonella LPS shared
epitopes (mimotopes)
SEQ ID No. 216: STLNYMYXAHPF-Core E. coli LPS)
SEQ ID No. 217: ISLSNIVDSQTP-LPS
(S. typhi and E. coli)
SEQ ID No. 218: GFSVITGAAMFE-LPS core and
Lipid A S. urbana and E. coli)
Combining these and other epitopes provides
vaccines and antibodies to important pathogens:
1. Malaria and HIV 2. Gram-positive and Gram-
negative bacteria and prevention or treatment of
sepsis and shock 3. H. influenza and Gram-
positive vaccine 4. Malaria, TB and HIV
5. Multi epitope TB vaccine

Combination Microbial Composite Peptides
Vaccine Antigens to Key Pathogens
1. malaria/HIV1/T
cell Epitopes-SEQ ID No. 219:
NPDPNANPNVDPNANGGGCRKSIHLGPGRAFYQYIKANSK
FIGITE
2. LPS/LTA/PGN/T
Cell epitopes-SEQ ID No. 220:
STLNYMYXAHPFWRMYFSHRHAHLRSPGGGGGAEK
AQYIKANSKFIGITE
3. LOS H. Flu/PGN/T
Cell epitopes-SEQ ID No. 221:
NMMRFTSQPPNNGGGGGAEKAQYIKANSKFIGITE
4. MALARIA/TB LAM/HIV3/T
Cell epitopes-SEQ ID No. 222:
NPDPNANPNVDPNANGGGCHSFKWLDSPRLRRKSIRIGPG
QAFYQYIKANSKFIGITE
5. PGN/TB LAM and TB 16.3
HSP/T cell epitopes-SEQ ID No. 223:
AEKAGGGGGHSFKWLDSPRLRSEFAYGSFVRTVSLPVGAD
EQYIKANSKFIGITE

### Example 9 Composite Peptide Vaccines for Targeting the N-Terminal Domain (NTD) of SARS-CoV-2 and Other Coronaviruses

The N-Terminal Domain (NTD) peptide sequences shown in Table 6 could be used to build composite peptide vaccine antigens with, or without a T-cell epitope (T-cell epitope combined sequences (SEQ ID NO 192) and with one or more coronavirus, influenza, or other microbe epitopes (SEQ ID NO 193). Highly conserved spike protein receptor binding domain (RBD) epitope with NTD: SEQ ID NO 190; SEQ ID NO 194. Coronavirus polymerase epitope with influenza M2e and Neuraminidase (NA) epitopes, combined with NTD: SEQ ID NO 192; SEQ ID NO 195. Coronavirus polymerase with influenza M2e and 2 highly conserved coronavirus spike protein RBD epitopes and a T cell epitope: SEQ ID NO 196. Coronavirus polymerase with 2 coronavirus highly conserved spike protein RBD epitopes, a coronavirus NTD epitope and a T cell epitope.

TABLE 6

| N-Terminal Domain (NTD) of SARS-COV-2 and other | | |
|---|---|---|
| coronaviruses sequences. | | |
| SEQ | | |
| ID | Peptide | |
| NO | number | Peptide Sequence |
| 178 | NTD | CATGIAVAG |
| | Pep01 | |
| 179 | NTD | YYYYYGMDVW |
| | Pep02 | |
| 180 | NTD | CATGYSSSWYFDYW |
| | Pep03 | |
| 181 | NTD | CAKGYSYGYNWFDSW |
| | Pep04 | |
| 182 | NTD | CQQYNNWPPLTF |
| | Pep05 | |
| 183 | NTD | CATGIAVAGQYIKANSKFIGITE |
| | Pep06 | |
| 184 | NTD | YYYYYGMDVWQYIKANSKFIGITE |
| | Pep07 | |
| 185 | NTD | CATGYSSSWYFDYWQYIKANSKFIGITE |
| | Pep08 | |
| 186 | NTD | CAKGYSYGYNWFDSWQYIKANSKFIGITE |
| | Pep09 | |
| 192 | NTD | CQQYNNWPPLTFQYIKANSKFIGITE |
| | Pep10 | |
| 193 | NTD | ARDLICAQCATGYSSSWYFDYWQYIKANSKFIGITE |
| | Pep11 | |
| 194 | NTD | WDYPKCDRATEVETPIRNEHYEECSCYCQQYNNWPPLTF |
| | Pep12 | QYIKANSKFIGITE |
| 195 | NTD | WDYPKCDRATEVETPIRNEARDLICAQENQKLIANCAT |
| | Pep13 | GIAVAGQYIKANSKDIGITE |
| 196 | NTD | WDYPKCDRAENQKLIANARDLICAQCATGYSSSWYFDY |
| | Pep14 | WQYIKANSKFIGITE |

### Example 10

To improve vaccine efficiency and global uptake of vaccines composite peptide vaccine antigens can combine multiple microbial peptide sequences to include, but not limited to peptide sequences that target respiratory viruses, hemorrhagic fever viruses, HIV, parasitic infections like malaria, bacterial infections, such as staphylococcus and M. tuberculosis and fungi such as candida, or aspergillosis. Composite peptide vaccine antigens could include, but are not limited to Malaria and HIV (1), gram negative and gram positive bacteria/toxins (2), gram negative and gram positive bacteria (3), malaria, TB and HIV (4), gram positive bacteria and TB (5). Composite peptide vaccine antigens can be combined using viral, bacterial, or parasitic peptide sequences in any order with, or without a T cell epitope. In addition, the composite peptide vaccine antigens can be given individually, or one or more composite peptide vaccines may be added together to further broaden the microbes targeted. Antibodies that target these composite peptides would be useful to prevent or treat the microbes that contain the epitopes within the composite peptides.

### Example 11 IgM Monoclonal Antibodies Targeting Peptidoglycan May Provide Therapeutic Strategies Against Antimicrobial Resistant Bacteria

Antimicrobial resistance (AMR) poses a substantial global threat to human health and development. In addition to death and disability, the cost of AMR to the global economy is significant. Prolonged illness results in longer hospital stays and the need for more expensive medicines and financial challenges for those impacted. Therapeutics such as monoclonal antibodies (mAbs) may offer prevention and control measures against microbial infections without the use of antibiotics. In this study, we developed human antibodies (serum and mAbs) against components of Staphylococcus aureus (SA) and Mycobacterium tuberculosis (MTB) and evaluated their capabilities.

Humanized DRAGA mice were immunized with 20 μg of a combination vaccine comprised of ultrapure peptidoglycan (PGN, derived from SA) and TB Pep01 peptide (targeting MTB HSP16.3), formulated with ADDAVAX™ adjuvant. Serum antibody responses to PGN, TB Pep01, and various whole bacteria were analyzed using ELISA. Mice with high antisera titers was selected for hybridoma production. Hybridomas were screened for binding to PGN, TB Pep01, and whole bacteria using ELISA and high producing clones were selected for monoclonal antibody development. Purified mAb was analyzed for recognition of live bacteria including Mycobacterium smegmatis, Staphylococcus epidermidis, and Staphylococcus aureus. Opsonophagocytic Killing Activity (OPKA) of purified mAb against live mycobacteria was assessed. Humanized DRAGA mice preferentially make IgM antibodies.

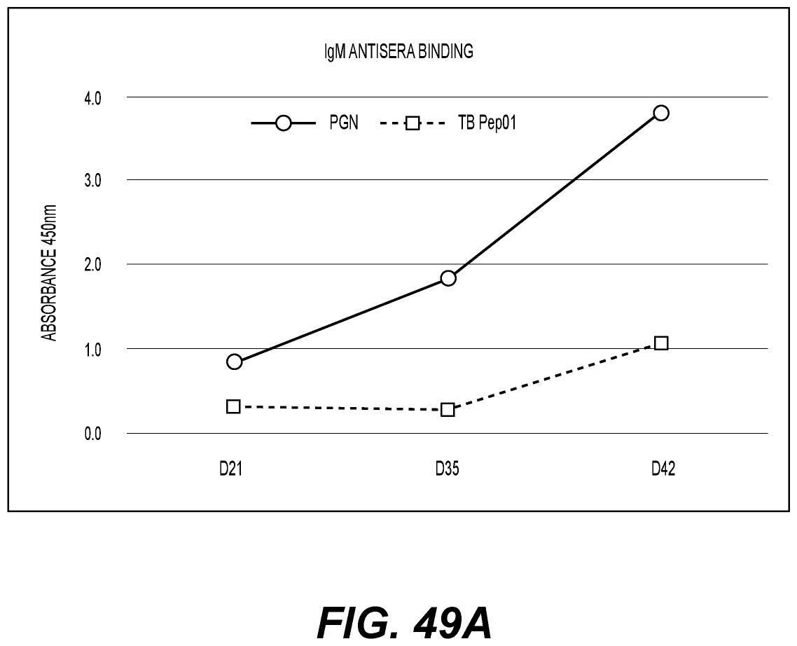

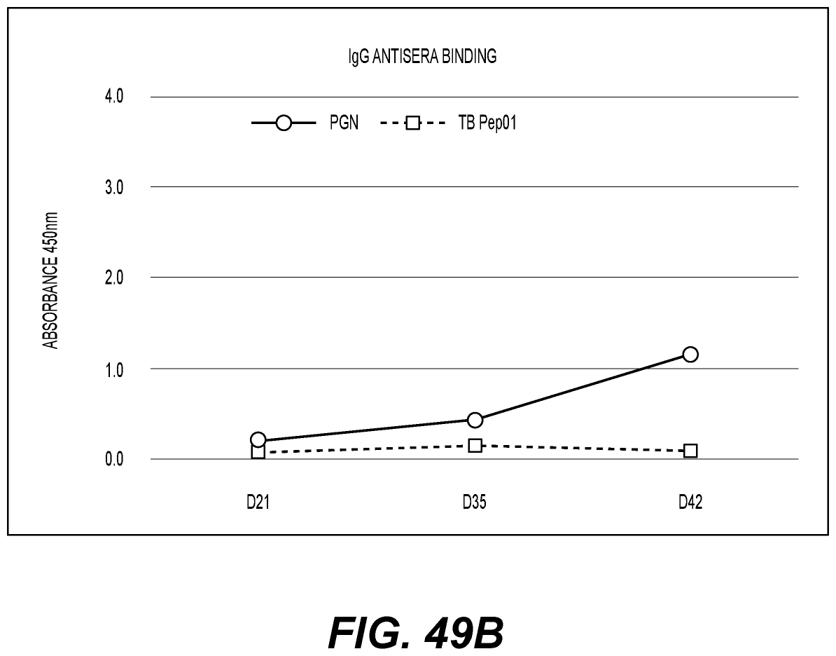

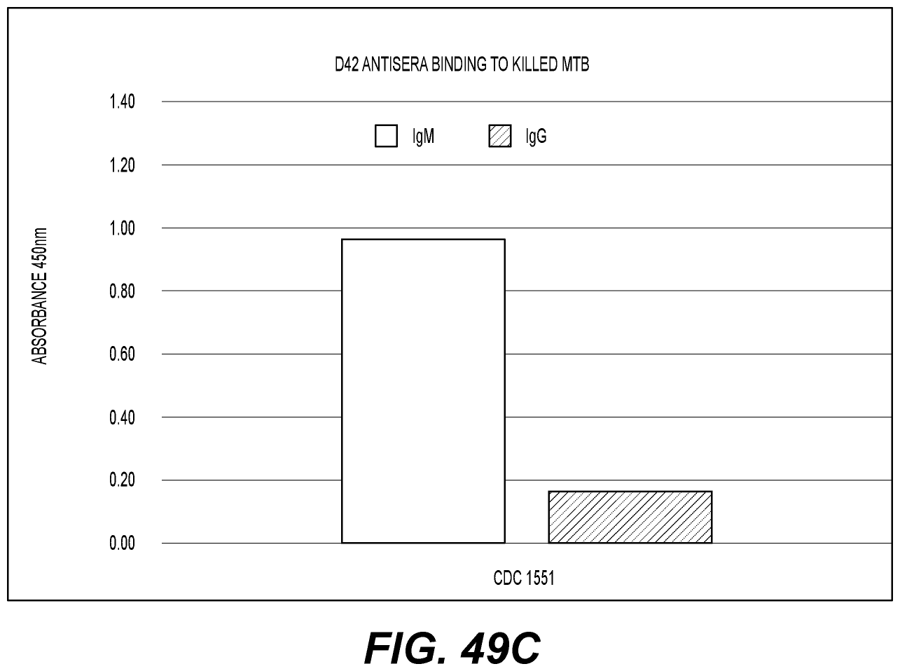

IgM monoclonal antibodies targeting peptidoglycan provide therapeutic strategies against antimicrobial resistant bacteria. Profiles of serum antibody responses to PGN and TB Pep 01 was analyzed using IgM (FIG. 49A) and IgG detection antibodies (FIG. 49B). Day-42 serum antibody responses to MTB CDC1551 is shown (FIG. 49C). Early and enhanced serum IgM responses to PGN were observed by Day-21 (FIG. 49A), while IgG responses to PGN were detected at Day-35 (FIG. 49B). Antisera binding to TB Pep01 was demonstrated, albeit lower than PGN. In addition, antisera recognition of whole bacteria was shown (FIG. 49C).

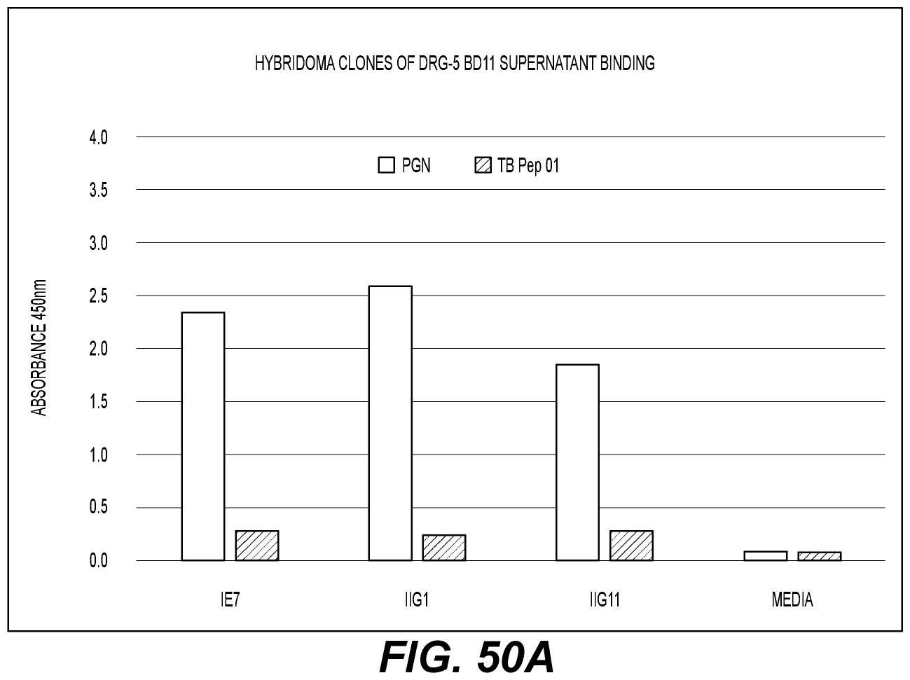

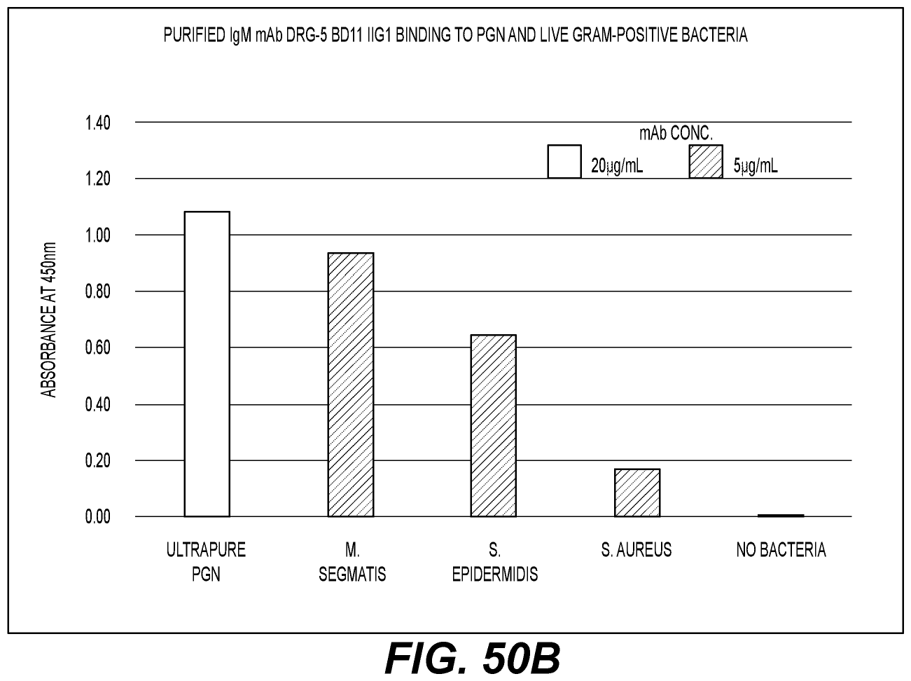

Hybridoma DRG-5BD11 clones (IgM) targeting PGN were identified for monoclonal antibody production (FIG. 50A). Purified IgM mAb DRG-5BD11 IIG1 bound to ultrapure PGN and to live gram-positive bacteria (FIG. 50B). Purified IgM mAb DRG-BD11 IIG1 show binding activity to PGN and various live gram-positive bacteria at 10\hat{ }5 CFU/mL and significantly enhanced killing of mycobacteria using U-937 macrophages (Table 7).

TABLE 7
Monoclonal Antibody Functional Activity

| mAb | Peak OPKA |
|---|---|
| 31 | 58% |
| 2 | 50% |
| 1 | 44% |
| 0.5 | 49% |

Preliminary functional activity of human IgM mAb DRG-5BD11I IG1 against mycobacteria (M. smegmatis) showed significant OPKA at 2 μg/mL and 31 μg/mL using U-937 macrophages, which has statistical significance of OPKA>50%.

Hybridomas developed in humanized DRAGA mice immunized with PGN and TBPep01 bound to the immunogens and showed broad recognition of various microbes. Ongoing studies to evaluate mAb functional activity against various microbes to include mycobacteria and staphylococci are in progress. IgM mAbs that recognize and whole bacteria, and opsonize and kill multiple bacterial strains, provide an effective antimicrobial strategy for treatment of drug resistant bacterial infections.

### Example 12

Many bacteria are becoming increasingly resistant to antibiotics that are essential for treating severe infections such as bacterial pneumonia and sepsis. Composite peptide vaccines that include multiple highly conserved epitopes, or mimotopes from gram negative (GN) and gram positive (GP) bacteria would be useful for preventing and treating infections caused by these antibiotic resistant bacteria. In addition, antibodies, (both polyclonal and monoclonal) would provide both prophylactic and therapeutic treatment options against these bacteria and could be used alone and in combination with antibiotics. Active and passive immunization to prevent wound (trauma related) infections and life-threatening sepsis and shock is of great value in high-risk patients especially those undergoing surgery, or immunosuppressive therapy. Epitopes and mimotopes are selected from a variety of molecules to include PGN, LTA, LPS and LPS core/Lipid A. Different microbial peptide epitopes are combined to produce composite peptide vaccines (e.g., Table 8). These composite peptides and antibodies to these epitopes are important as bacteria become broadly resistant to many classes of antibiotics.

TABLE 8
LPS Peptides, or mimotopes that interact with the TLR-4 receptor:

| SEQ ID NO | Sequence | Name | Epitope | Results |
|---|---|---|---|---|
| 197 | QEINSSY | (RS01) | LPS-T epitope | Good cytokine induction |
| 198 | APPHALS | (RS09) | LPS epitope | Good cytokine induction |
| 199 | VVPTPPY | (RS11) | LPS epitope | Activated NF-KB |
| 200 | SMPNPMV | (RS03) | LPS epitope | Activated NF-KB |
| 201 | GLQQVLL | (RS04) | LPS epitope | Not very soluble |
| 202 | ELAPDSP | (RS12) | LPS epitope | Activated NF-KB |

| SEQ ID NO | Sequence |
|---|---|
| 203 | QEINSSYQYIKANSKFIGITE (LPS-T epitope) |
| 204 | APPHALSQYIKANSKFIGITE (LPS-T epitope) |
| 205 | VVPTPPYQYIKANSKFIGITE (LPS-T epitope) |
| 206 | QEINSSYAEKAGGGGGWRMYFSHRHAHLRSPQYIKANSKFIGITE (LPS- |
| | PGN-LTA-T epitope) |
| 207 | APPHALSAEKAGGGGGWRMYFSHRHAHLRSPQYIKANSKFIGITE (LPS- |
| | PGN-LTA-T epitope) |
| 208 | VVPTPPYAEKAGGGGGWRMYFSHRHAHLRSPQYIKANSKFIGITE (LPS- |
| | PGN-LTA-T epitope) |
| 209 | AEAKAGGGGGWRMYFSHRHAHLRSPQEINSSYQYIKANSKFIGITE (PGN- |
| | LTA- LPS core/Lipid A-T epitope) |
| 210 | WRMYFSHRHAHLRSPAPPHALSAEKAGGGGGQYIKANSKFIGITE (LTA-LPS-PGN- |
| | T epitope) |
| 211 | QYIKANSKFIGITEWRMYFSHRHAHLRSAEKAGGGGGVVPTPPY (T-epitope-LTA- |
| | LPS-PGN-LPS) |
| 212 | QEINSSYAEKAGGGGGWRMYFSHRHAHLRSPGFSVITGAAMFE |
| | QYIKANSKFIGITE (LPS-PGN-LTA-LPS-T epitope) |
| 213 | SLLTEVETPIRNEWGLLTEVETPIRQYIKANSKFIGITE (Pep 5906; conserved matrix |
| | region (M1/M2e) plus T cell epitope) |

191 and 196 had activated NF-kB with adjuvant. Antibodies that bound to the LPS containing peptides similarly bound to LPS. Peptides RS01 and RS09 were analyzed in BALB/c mice—09 had adjuvant activity.

### Example 13 Adjuvanted Unconjugated Multi-Epitope Influenza Peptide Vaccine LHNVD-105 Study in Swine

Pigs (n=22) were injected IM with an immunogen comprised of GMP Flu Pep 5906 (SEQ ID NO 213; SLLTEVETPIRNEWGLLTEVETPIRQYIKANSKFIGITE (M1/M2/M2e conserved region with a universal T cell epitope) plus Pep 11 (SEQ ID NO 63); GNLFIAPWGVIHHPHYEECSCY) in ADDAVAX™ reconstituted in water (referred to as LHNVD-105) at 100 μg, 250 μg, or 500 μg, or with PBS as a negative control. Body weight increased from approximately 20 lb to approximately 80 lbs for all animals over the 49-day test period. Body temperatures varied during the test period between approximately 101° F. (39° C.) and approximately 103° F. (40° C.).

Animals were observed throughout the course of the study for any negative local or systemic side effects. Images were taken of injection sites two days post intramuscular immunization. No adverse reactions were observed post immunization in any treatment group. All pigs exhibited normal behavior without signs of distress or reactogenicity at the injection site after administration of the vaccine. Pigs were euthanized at the conclusion of the study.

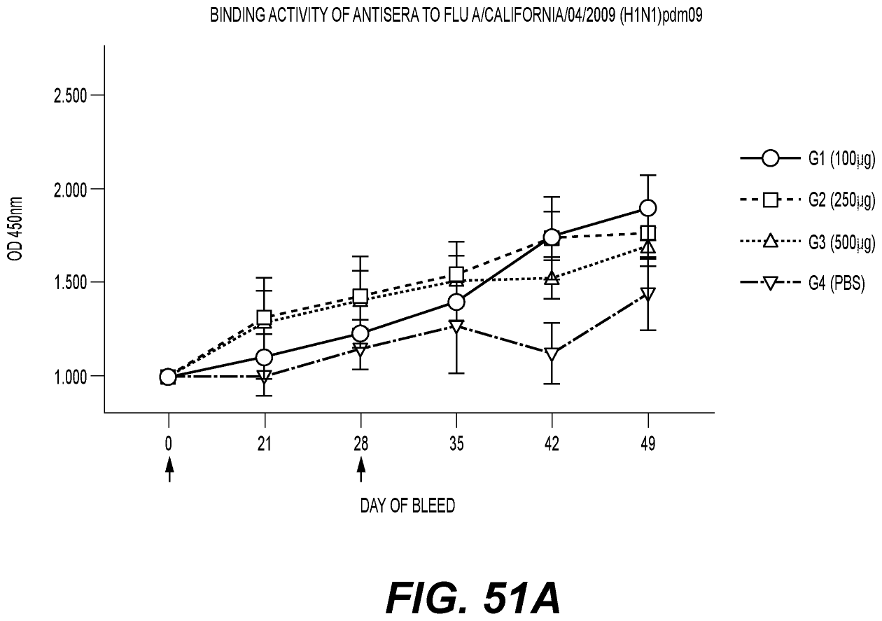

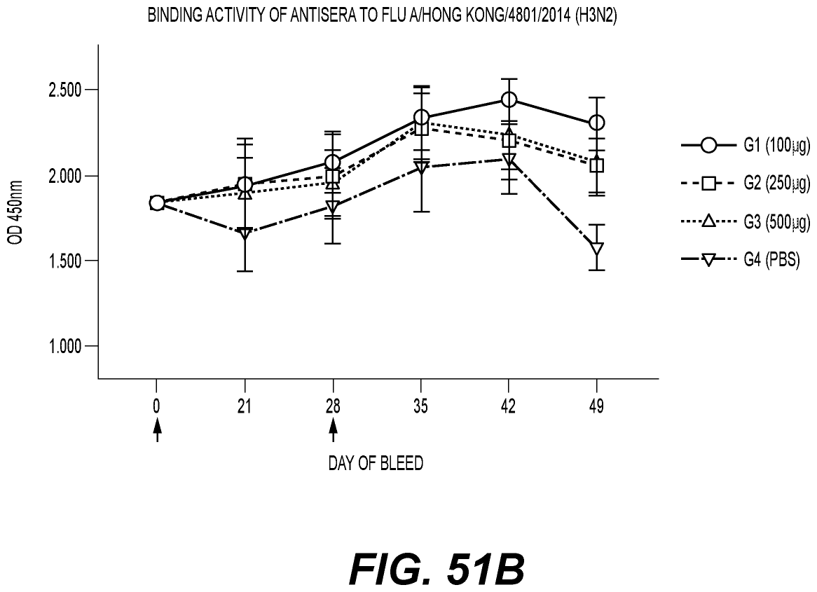

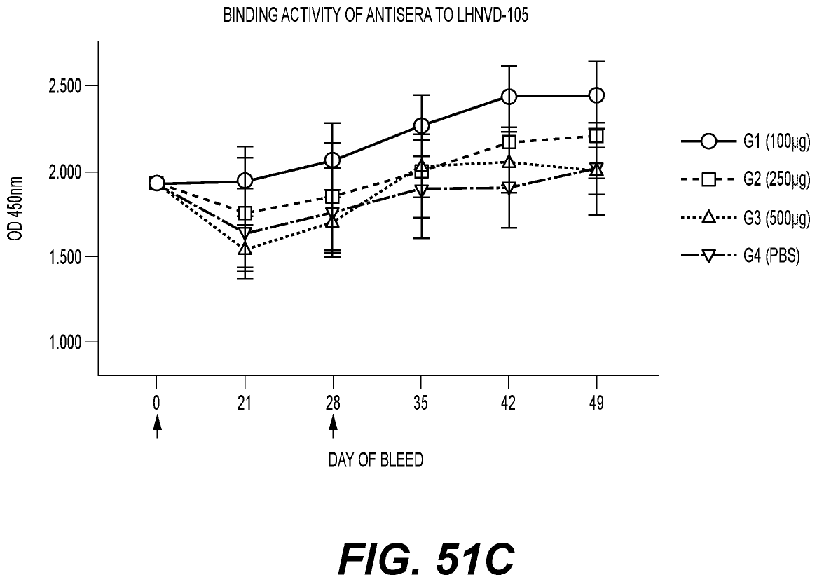

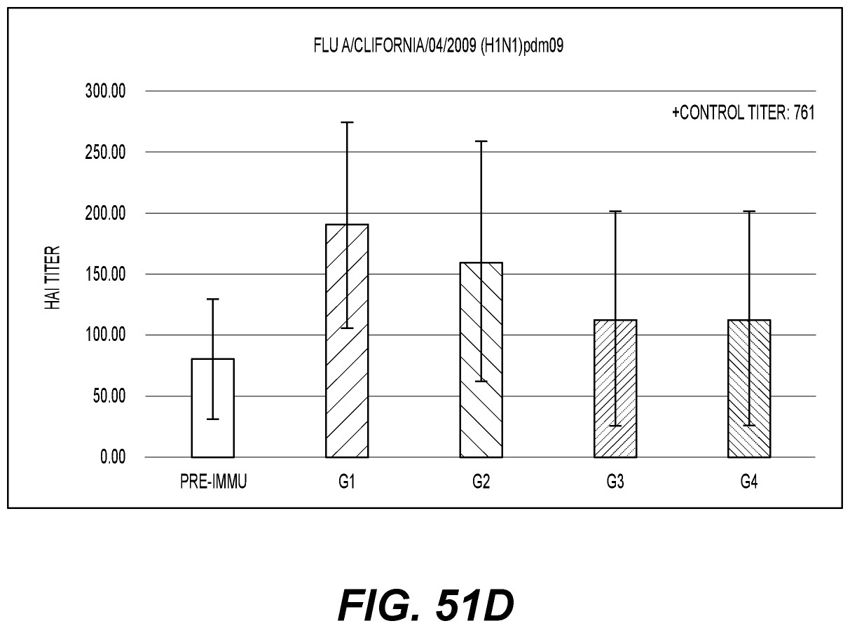

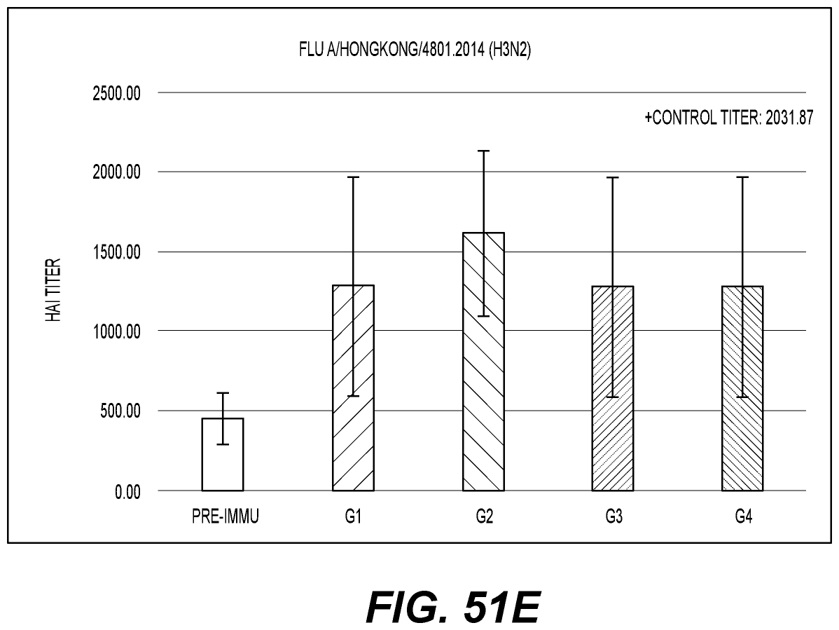

Binding activity of antisera from pigs immunized with LHNVD-105 at 100 μm, 250 μm, or 500 μm dose or PBS on d0 and d28 to: (i) whole virus of Flu A/California (H1N1)pdm09 (see FIG. 51A); (ii) whole virus of Flu A/Hong Kong/4801/2014 (H3N2) (see FIG. 51B); (iii) peptide LHNVD-105 (see FIG. 51C) are shown. Data are represented as means±SEM. Binding activity increased from 1.0 at OD450 (water) to 1.5 and almost 2.0 for the animals which received LHNVD-105 injections. Surprisingly, antisera from animals that received the lower quantities of peptide (100 μg and 250 μg) showed the greater binding to both whole viruses and the peptide LHNVD-105.

Functional assays were performed of HAI titers on animals injected with LHNVD-105 at 100 μg, 250 μg, or 500 μm dose or PBS on d49 against Flu A/California (H1N1)pdm09 (See FIG. 51D) and Flu A/Hong Kong/4801/2014 (H3N2) (see FIG. 51E). Once again, antisera from animals administered the lower doses of LHNVD-105 (100 μg and 250 μg) showed a greater HA titer as compared to animals administered the higher dose (500 μg), animals administered PBS only, and pre-immune animals.

Other embodiments and uses of the invention will be apparent to those skilled in the art from consideration of the specification and practice of the invention disclosed herein. All references cited herein, including all publications and U.S. and foreign patents and patent applications, are specifically and entirely incorporated by reference including U.S. Patent Publication No. 20210246174A1 entitled “Immunogenic Compositions to Treat and Prevent Microbial Infections”, published Aug. 12, 2021, U.S. Pat. No. 9,821,047 entitled “Enhancing Immunity to Tuberculosis,” which issued Nov. 21, 2017, U.S. Pat. No. 9,598.462 entitled “Composite Antigenic Sequences and Vaccines” which issued Mar. 21, 2017, U.S. Pat. No. 10,004,799 entitled “Composite Antigenic Sequences and Vaccines” which issued Jun. 26, 2018, U.S. Pat. No. 8,652,782 entitled “Compositions and Method for Detecting, Identifying and Quantitating Mycobacterial-Specific Nucleic Acid, ” which issued Feb. 18, 2014, U.S. Pat. No. 9,481,912 entitled “Compositions and Method for Detecting, Identifying and Quantitating Mycobacterial-Specific Nucleic Acid, ” which issued Nov. 1, 2016, U.S. Pat. No. 8,821,885 entitled “Immunogenic Compositions and Methods,” which issued Sep. 2, 2014, and all corresponding U.S. Provisional and continuation applications relating to any of the foregoing patents. The term comprising, where ever used, is intended to include the terms consisting of, and consisting essentially of. Furthermore, the terms comprising, including, containing and the like are not intended to be limiting. It is intended that the specification and examples be considered exemplary only with the true scope and spirit of the invention indicated by the following claims.

1. A composition comprising an immunogenic portion of a peptidoglycan and an immunogenic portion of a heat shock protein.

2. The composition of claim 1, wherein the immunogenic portion of the peptidoglycan is obtained from a gram-positive microorganism.

3. The composition of claim 2, wherein the gram-positive microorganism is of a spp. of Mycobacteria, a spp. of Staphylococcus, a spp. of Bacillus, or a spp. of Streptococcus.

4. The composition of claim 1, wherein the immunogenic portion of the peptidoglycan comprises multiple immunogenic portions of a peptidoglycan protein.

5. The composition of claim 4, wherein the multiple immunogenic portions of the peptidoglycan protein comprise substantially all of the peptidoglycan protein.

6. The composition of claim 1, wherein the immunogenic portion of the heat shock protein is of a spp. of Mycobacteria.

7. The composition of claim 6, wherein the spp. of Mycobacteria is M. tuberculosis, M. smegmatis, M. leprae, M. kansasii, M. mantenii, M. fortuitum, or M. xenopi.

8. The composition of claim 1, wherein the immunogenic portion of the heat shock protein comprises an immunogenic portion of an alpha helix portion of the heat shock protein.

9. The composition of claim 1, wherein the immunogenic portion of the heat shock protein comprises an alpha helix portion of a spp. of Mycobacteria and the immunogenic portion of the peptidoglycan comprises the peptidoglycan protein of a spp. of Mycobacteria, a spp. of Staphylococcus, a spp. of Bacillus, or a spp. of Streptococcus.

10. The composition of claim 1, wherein the immunogenic portion of peptidoglycan and the immunogenic portion of the heat shock protein are a contiguous amino acid sequence.

11. The composition of claim 10, wherein the contiguous sequence comprises the sequence of SEQ ID NOs 148, 149, 151, or 152.

12. The composition of claim 1, further comprising a T cell stimulating epitope of tetanus toxin, tetanus toxin heavy chain proteins, diphtheria toxoid, CRM, recombinant CRM, tetanus toxoid, Pseudomonas exoprotein A, Pseudomonas aeruginosa toxoid, Bordetella pertussis toxoid, Clostridium perfringens toxoid, Escherichia coli heat-labile toxin B subunit, Neisseria meningitidis outer membrane complex, Hemophilus influenzae protein D, Flagellin Fli C, Horseshoe crab Haemocyanin, and/or a fragment, derivative, or modification thereof.

13. The composition of claim 12, wherein the T cell stimulating epitope is a contiguous amino acid sequence with the immunogenic portion of the peptidoglycan and/or the immunogenic portion of the heat shock protein.

14. The composition of claim 13, wherein the contiguous amino acid sequence comprises the sequence of SEQ ID NOs 150 or 153.

15. The composition of claim 1, further comprising an adjuvant.

16. The composition of claim 15, wherein the adjuvant comprises Freund's adjuvant, ALFQ, ALFQA, ALFA, AS01, AS01b, a liposome adjuvant, saponin, lipid A, squalene, and/or modifications, emulsions, nano-emulsions, derivatives, and combinations thereof.

17. The composition of claim 1, which treats or prevents a Mycobacterial infection in a mammal.

18. The composition of claim 1, which treats or prevents infection of a gram-positive microorganism in a mammal.

19. A vaccine comprising the composition of claim 1.

20. A composition comprising an immunogenic portion of a peptidoglycan of a gram-positive microorganism, an immunogenic portion of a heat shock protein of a spp. of Mycobacteria, and an adjuvant in the form of a nano-emulsion.

21. The composition of claim 20, further comprising a T cell stimulating epitope.

22. The composition of claim 21, wherein the T cell stimulating epitope is an amino acid sequence that is contiguous with the sequence of the immunogenic portion of the peptidoglycan and/or the sequence of the immunogenic portion of the heat shock protein.

23. A method of treating or preventing an infection in a mammal by administering the composition of claim 1 to the mammal.

24. The method of claim 23, wherein administration is oral, sub-cutaneous, intra-muscular, intradermal, or intra-nasal.

25. The method of claim 23, wherein administration produces a systemic or mucosal immune response against the infection.

26. The method of claim 23, wherein administration produces antibodies that provide an opsonophagocytic response to the infection.

27. Antibodies that bind to the immunogenic portion of peptidoglycan and the immunogenic portion of the heat shock protein of claim 1.

28. The antibodies of claim 27, which comprises IgG, IgA, IgD, IgE, IgM, or fragments or combinations thereof.

29. The antibodies of claim 27, which induce opsonophagocytic killing activity against a microorganism.

30. The antibodies of claim 29, wherein the microorganism comprises a spp. of Mycobacteria, a spp. of Staphylococcus, a spp. of Bacillus, or a spp. of Streptococcus.

31. The antibodies of claim 27, which are polyclonal, monoclonal, or humanized.

32. A hybridoma that expresses the monoclonal antibodies of claim 31.

33. A hybridoma which comprise cells of ATCC deposit number PTA-127658.

34. Antibodies expressed from the cells of the hybridoma of claim 33.
